(19) United States
(12) Patent Application Publication
(10) Pub. No.: US 2010/0197711 A1
(43) Pub. Date: Aug. 5, 2010

(54) BENZOTHIAZOLE COMPOUNDS

(76) Inventors: Keith Geoffrey Watson, Victoria (AU); Guillaume Laurent Lessene, Victoria (AU); Jonathan Bayldon Baell, Victoria (AU); David Ching Siang Huang, Victoria (AU); Ian Philip Street, Victoria (AU); Jerry McKee Adams, Victoria (AU); Peter Malcolm Colman, Victoria (AU); Brad Edmund Sleebs, Victoria (AU); Brian John Smith, Victoria (AU); Peter Edward Czabotar, Victoria (AU)

Correspondence Address: WILMERHALE/BOSTON, 60 STATE STREET, BOSTON, MA, 02109, US

(21) Appl. No.: 12/678,836

(22) PCT Filed: Sep. 27, 2007
(86) PCT No.: PCT/AU07/01442

§ 371 Date: Mar. 18, 2010

(51) Int. Cl. A61K 31/428; C07D 277/82; C07D 417/12; C12N 5/09; A61K 31/506; A61K 31/4439; A61P 29/00; A61P 35/00; A61P 37/00; A61P 35/02; A61P 19/02

(52) U.S. Cl. 514/274; 548/161; 544/316; 546/270.1; 435/375; 514/367; 514/338

(57) ABSTRACT

The present invention relates to benzothiazole compounds that mimic the activity of BH3 only proteins and are capable of binding to and neutralizing pro survival Bcl 2 proteins. The invention also relates to the use of such compounds in the regulation of cell death or cell survival and the treatment and/or prophylaxis of diseases or conditions associated with the deregulation of cell death or cell survival.

### FIELD OF THE INVENTION

The invention relates generally to small molecules that mimic the biological activity of certain peptides and proteins, to compositions containing them and to their use. In particular, the invention relates to benzothiazole compounds that mimic the biological activity of BH3-only proteins and are capable of binding to and neutralising pro-survival Bcl-2 proteins. The invention also relates to processes of preparing the benzothiazole compounds that mimic portions of peptides and proteins, and to the use of such compounds in the regulation of cell death or cell survival and the treatment and/or prophylaxis of diseases or conditions associated with the deregulation of cell death or cell survival.

### BACKGROUND OF THE INVENTION

Bibliographical details of various publications referred to in this specification are collected at the end of the description.

The reference to any publication in this specification is not, and should not be taken as, an acknowledgment or any form of suggestion that that publication forms part of the common general knowledge in Australia.

Apoptosis is now recognized as an essential biological process in the tissue homeostasis of all living species. In mammals in particular, it has been shown to regulate embryonic development. Later in life, cell death is a default mechanism that removes potentially dangerous cells (e.g. cells carrying cancerous defects). Several apoptotic pathways have been uncovered and one of the most important involves the Bcl-2 family of proteins. The structural homology domains BH1 to BH4 are characteristic of this family. Further classification into of three subfamilies depends on how many of these homology domains a protein contains and on its biological activity (pro- or anti-apoptotic).

The first subgroup contains proteins having all 4 homology domains BH1 to BH4. Their general effect is anti-apoptotic thus preserving the cell from starting a cell death process. Proteins such as Bcl-2, Bcl-w, Bcl-x_{L}, Mcl-1 and Bfl-1/A1 are members of this first subgroup. Proteins belonging to the second subgroup have a pro-apoptotic effect and contain the three homology domains BH1 to BH3. The two main representative proteins of this second subgroup are Bax and Bak. Finally, the third subgroup is composed of protein containing only the BH3 domain and members of this subgroup are usually referred to as “BH3-only proteins”. Their biological effect on the cell is pro-apoptotic. Bim, Bad, Bmf, and Bid are examples of this third subfamily of proteins.

The delicate balance between the three subgroups is the key to homeostasis of the cells. Recent studies have tried to elucidate the mechanisms involving the Bcl-2 family of proteins that allow a cell to undergo programmed cell death upon receiving intra- or extra-cellular signal. Such a signal induces the activation (post translational or transcriptional) of BH3-only proteins. These proteins are the primary inducers of the cascade that leads to cell death. The BH3-only proteins mainly interact with the Bcl-2 subgroup and stop proteins such as Bcl-2, Bcl-x_{L} or Bcl-w from inhibiting the Bax/Bak subgroup. These later proteins are either already anchored to the mitochondrial membrane or migrate to this membrane. Their activation leads to membrane swelling, release of cytochrome C and downstream activation of effector caspases resulting in apoptosis.

As already mentioned the balance between these proteins is essential to the correct cellular response to various stimuli. Any perturbation of this balance will instigate or worsen major diseases. Thus apoptosis perturbations have been shown to be at the origin of important diseases such as neurodegenerative conditions (up-regulated apoptosis) for example, Alzheimer's disease, or proliferative diseases (down-regulated apoptosis) for example, cancer and autoimmune diseases.

The discovery that several proteins of the Bcl-2 family are involved in the onset of cancerous malignancy has unveiled a completely novel way of targeting this still elusive disease. It has been shown in particular that pro-survival proteins such as Bcl-2 are over-expressed in many cancer types (see Table 1) [Zhang, 2002]. The effect of this deregulation is the survival of altered cells which would have undergone apoptosis in normal conditions. The repetition of these defects associated with unregulated proliferation is thought to be the starting point of cancerous evolution. BH3-only proteins have also been shown to act as tumor suppressors when their expression is attenuated in diseased animals.

TABLE 1
Bcl-2 over-expression in cancer

| Cancer type | Bcl-2 over-expression |
|---|---|
| Hormone-refractory | 90-100% |
| prostate cancer | |
| Malignant melanoma | 90% |
| Oestrogen-receptor- | 80-90% |
| positive breast cancer | |
| Non-Hodgkin's | 50% |
| lymphoma | |
| Colon Cancer | 30-50% |
| Chronic lymphocytic | 25-50% |
| leukaemia | |

These findings as well as numerous others have made possible the emergence of new concept in anti-cancer strategies and drug discovery. If an entity mimicking the effect of BH3-only proteins were able to enter the cell and overcome the pro-survival protein over-expression, it could be possible to reset the apoptotic process. This strategy may have the advantage that it may alleviate the problem of drug resistance which is usually a consequence of apoptotic deregulation (abnormal survival).

A considerable effort has been made to understand the structural details of the key interactions between BH3-only proteins and the pro-survival subgroup. Fesik and co-workers have demonstrated in the case of the dimer Bad/Bcl-x_{L} the importance of some structural elements [Muchmore et. al., 1996; Sattler et. al., 1997 and Petros et. al., 2000]:
Binding occurs between a hydrophobic groove located on Bcl-x_{L} and the BH3 domain of Bad.
The BH3-only protein Bad adopts a helix structure upon binding to the hydrophobic groove of Bcl-x_{L}.
Four hydrophobic amino-acids of the BH3 domain located at i, i+3, i+7 and i+11 intervals are essential to the binding of Bad to Bcl-x_{L} and interact in four hydrophobic pockets situated in the Bcl-x_{L} binding groove. Moreover, studies of members of the BH3-only subgroups have shown that these four hydrophobic amino-acids are conserved through the subgroup.

Recently the structure of the pro-survival protein Bcl-w [Hinds et. al., 2003] and the structure of BH3-only protein Bim in interaction with Bcl-x_{L} [Liu et. al., 2003] have been published. This latter structure confirms the findings of the Bad/Bcl-x_{L} interaction.

A potential target for new drug therapy is small molecules that mimic the interaction between a BH3-only protein and the Bcl-2 family of proteins.

Recently a small molecule BH3-only protein mimetic has been shown to have cytotoxic activity in some cancer cell lines and to enhance the effects of radiation therapy and a number of chemotherapeutic agents [Oltersdorf et. al., 2005; US 2002/0086887; WO 03/080586; U.S. Pat. No. 6,720,338; WO 05/049597; Cory and Adams, 2005].

The alpha-helix is a common recognition motif displayed in peptides and proteins. Alpha-helical sequences are often involved in protein-protein interactions, such as enzyme-receptor and antibody-receptor interactions. Targeting these protein-protein interactions is now recognised as one of the major challenges in drug discovery.

There is a need for small molecules which may be easily synthesised and that mimic the activity of BH3-only proteins.

### SUMMARY OF THE INVENTION

The present invention is predicated in part on the discovery that benzothiazole derivatives provide a BH3-only protein mimetic which is able to interact with a Bcl-2 protein. This discovery has been reduced to practice in novel compounds, compositions containing them and in methods for their preparation and use, as described hereafter.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

In a first aspect of the invention, there is provided a compound of formula (I):

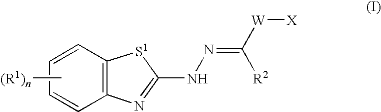

wherein

S¹ is S or Se;

W is an aryl or heteroaryl group;

X is -L-Z or -Q-Y—Z;

L is a linker of 1 to 3 atoms in length;

Q is an aryl or heteroaryl group;

Y is absent or is C₁₋₂alkylene;

Z is a carboxy group or an optionally substituted isosteric equivalent of a carboxy group;

R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyano and —N(R⁵)₂;

R² is selected from the group consisting of C₁₋₇alkyl, C₂₋₇alkenyl, C₂₋₇alkynyl, (CH₂)_{q}C₃₋₈cycloalkyl, (CH₂)_{q}halo, (CH₂)_{q}OH, C₁₋₇alkoxy(CH₂)ₜ, C₂₋₇alkenyloxy(CH₂)ₜ, C₂₋₇alkynyloxy(CH₂)ₜ, C₃₋₈cycloalkyloxy(CH₂)ₜ, C₁₋₇alkylthio(CH₂)ₜ, C₂₋₇alkenylthio(CH₂)ₜ, C₂₋₇alkynylthio(CH₂)ₜ, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)₁M(CH₂)ₜheterocyclyl,

or R² together with a ring atom from W forms an optionally substituted 5-8 membered carbocyclic or heterocyclic ring;

each R⁴ is independently selected from the group consisting of hydrogen and halogen;

each R⁵ is independently selected from the group consisting of hydrogen and C₁₋₃alkyl;

each R⁶ is independently selected from the group consisting of hydrogen, C₁₋₇alkyl, C₂₋₇alkenyl, C₂₋₇alkynyl, C₃₋₈cycloalkyl, acyl, aryl, heterocyclyl or heteroaryl, or two R⁶ taken together with the nitrogen atom to which they are attached form an optionally substituted carbocyclic or heterocyclic ring;

R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃₋₈cycloalkyloxy, aryloxy, heteroaryloxy, heterocyclyloxy and N(R⁶)₂;

M is O, S or NR⁵;

n is 0, 1 or 2;

q is 1 to 7;

r is 1 to 4;

t is 0 or 1 to 4;

wherein each alkyl, alkenyl, alkynyl, alkylene, linker, carbocyclic, heterocyclic, aryl and heteroaryl group may be optionally substituted with one or more optional substituents;

and salts, esters and isomers thereof;

provided that said compound is other than [4-[1-[(6-ethoxy-2-benzothiazolyl)hydrazono]ethyl]phenoxy]-acetic acid; [4-[1-[(6-methoxy-2-benzothiazolyl)hydrazono]ethyl]phenoxy]-acetic acid; [4-[1-[(6-methyl-2-benzothiazoly)hydrazono]ethyl]phenoxy]-acetic acid; [4-[1-[(6-chloro-2-benzothiazolyl)hydrazono]ethyl]phenoxy]acetic acid; and [4-[1-[(2-benzothiazolylhydrazono)ethyl]phenoxy]-acetic acid.

As used herein, the term “alkyl” refers to a straight chain or branched saturated hydrocarbon group having 1 to 20 carbon atoms. Where appropriate, the alkyl group may have a specified number of carbon atoms, for example, C₁₋₆alkyl which includes alkyl groups having 1, 2, 3, 4, 5 or 6 carbon atoms in a linear or branched arrangement. Examples of suitable alkyl groups include, but are not limited to, methyl, ethyl, n-propyl, i-propyl, n-butyl, i-butyl, t-butyl, n-pentyl, 2-methylbutyl, 3-methylbutyl, 4-methylbutyl, n-hexyl, 2-methylpentyl, 3-methylpentyl, 4-methylpentyl, 5-methylpentyl, 2-ethylbutyl, 3-ethylbutyl, heptyl, octyl, nonyl, decyl and dodecyl.

As used herein, the term “alkenyl” refers to a straight-chain or branched hydrocarbon group having one or more double bonds between carbon atoms and having 1 to 20 carbon atoms. Where appropriate, the alkenyl group may have a specified number of carbon atoms. For example, C₂-C₆ as in “C₂-C₆alkenyl” includes groups having 2, 3, 4, 5 or 6 carbon atoms in a linear or branched arrangement. Examples of suitable alkenyl groups include, but are not limited to, ethenyl, propenyl, isopropenyl, butenyl, butadienyl, pentenyl, pentadienyl, hexenyl, hexadienyl, heptenyl, octenyl, nonenyl, decenyl and dodecenyl.

As used herein, the term “alkynyl” refers to a straight-chain or branched hydrocarbon group having one or more triple bonds between carbon atoms and having 1 to 20 carbon atoms. Where appropriate, the alkynyl group may have a specified number of carbon atoms. For example, C₂-C₆ as in “C₂-C₆alkynyl” includes groups having 2, 3, 4, 5 or 6 carbon atoms in a linear or branched arrangement. Examples of suitable alkynyl groups include, but are not limited to, ethynyl, propynyl, butynyl, pentynyl, hexynyl, octynyl, nonynyl, decynyl and dodecynyl.

As used herein, the term “carbocyclic” or “carbocyclyl” refers to a cyclic hydrocarbon that may be saturated or unsaturated. The carbocyclic ring may include a specified number of carbon atoms. For example, a 5 to 8 membered carbocyclic ring includes 5, 6, 7 or 8 carbon atoms. Examples of suitable carbocyclic rings include, but are not limited to, cyclopentanyl, cyclopentenyl, cyclohexanyl, cyclohexenyl, cycloheptanyl, cycloheptenyl, cycloheptadienyl, cycloheptatrienyl, cyclooctanyl, cyclooctenyl, cyclooctadienyl and cyclooctatrienyl rings.

As used herein, the term “cycloalkyl” refers to a saturated cyclic hydrocarbon. The cycloalkyl ring may include a specified number of carbon atoms. For example, a 3 to 8 membered cycloalkyl group includes 3, 4, 5, 6, 7 or 8 carbon atoms. Examples of suitable cycloalkyl groups include, but are not limited to, cyclopropyl, cyclobutyl, cyclopentanyl, cyclohexanyl, cycloheptanyl and cyclooctanyl.

The terms “alkyloxy” or “alkoxy”, “alkenyloxy” and “alkynyloxy” as used herein represent an alkyl, alkenyl or alkynyl group as defined above attached through an oxygen bridge. Examples of suitable alkyloxy, alkenyloxy and alkynyloxy groups include, but are not limited to, methoxy, ethoxy, n-propyloxy, n-butyloxy, n-pentyloxy, n-hexyloxy, ethenyloxy, propenyloxy, butenyloxy, pentenyloxy, hexenyloxy, ethynyloxy, propynyloxy, butynyloxy, pentynyloxy and hexynyloxy.

The terms “alkylthio”, “alkenylthio” and “alkynylthio” as used herein represent an alkyl, alkenyl or alkynyl group as defined above attached through a sulfur bridge. Examples of suitable alkylthio, alkenylthio and alkynylthio include, but are not limited to, methylthio, ethylthio, propylthio, butylthio, pentylthio, hexylthio, ethenylthio, propenylthio, butenylthio, pentenylthio, hexenylthio, ethynylthio, propynylthio, butynylthio, pentynylthio and hexynylthio.

The term “acyl” used herein refers to an alkanoyl or aroyl group as defined by (C═O)R^{a} where suitable R^{a} groups include, but are not limited to, C₁₋₇alkyl, C₁₋₇alkenyl, C₁₋₇alkynyl, C₃₋₈cycloalkyl, aryl, heterocyclyl, heteroaryl, C₁₋₇alkylaryl, C₁₋₇alkylcycloalkyl, C₁₋₇alkylheterocyclyl, C₁₋₇alkylheteroaryl, C₁₋₇alkoxyalkyl, C₁₋₇alkylthioalkyl, C₁₋₇alkylthioaryl, C₁₋₇alkoxyaryl and the like.

As used herein, the term “aryl” is intended to mean any stable, monocyclic or bicyclic carbon ring of up to 7 atoms in each ring, wherein at least one ring is aromatic. Examples of such aryl groups include, but are not limited to, phenyl, naphthyl, tetrahydronaphthyl, indanyl, biphenyl and binaphthyl.

As used herein, the term “halogen” or “halo” refers to fluorine (fluoro), chlorine (chloro), bromine (bromo) and iodine (iodo).

The term “heterocyclic” or “heterocyclyl” as used herein, refers to a cyclic hydrocarbon in which one to four carbon atoms have been replaced by heteroatoms independently selected from the group consisting of N, N(R), S, S(O), S(O)₂ and O. A heterocyclic ring may be saturated or unsaturated. Examples of suitable heterocyclyl groups include tetrahydrofuranyl, tetrahydrothiophenyl, pyrrolidinyl, pyrrolinyl, pyranyl, piperidinyl, pyrazolinyl, dithiolyl, oxathiolyl, dioxanyl, dioxinyl, morpholino and oxazinyl.

The term “heteroaryl” as used herein, represents a stable monocyclic or bicyclic ring of up to 7 atoms in each ring, wherein at least one ring is aromatic and at least one ring contains from 1 to 4 heteroatoms selected from the group consisting of O, N and S. Heteroaryl groups within the scope of this definition include, but are not limited to, acridinyl, carbazolyl, cinnolinyl, quinoxalinyl, quinazolinyl, pyrazolyl, indolyl, benzotriazolyl, furanyl, thienyl, thiophenyl, benzothienyl, benzofuranyl, benzodioxane, benzodioxin, quinolinyl, isoquinolinyl, oxazolyl, isoxazolyl, imidazolyl, pyrazinyl, pyridazinyl, pyridinyl, pyrimidinyl, pyrrolyl, tetrahydroquinoline, thiazolyl, isothiazolyl, 1,2,4-triazolyl, 1,2,4-oxadiazolyl, 1,2,4-thiadiazolyl, 1,3,5-triazinyl, 1,2,4-triazinyl, 1,2,4,5-tetrazinyl and tetrazolyl. Particular heteroaryl groups have 5- or 6-membered rings, such as pyrazolyl, furanyl, thienyl, oxazolyl, isoxazolyl, imidazolyl, pyrazinyl, pyridazinyl, pyridinyl, pyrimidinyl, pyrrolyl, thiazolyl, isothiazolyl, 1,2,4-triazolyl and 1,2,4-oxadiazolyl and 1,2,4-thiadiazolyl.

Each alkyl, alkenyl, alkynyl, cycloalkyl, carbocyclyl, alkylene, alkenylene, aryl, heterocyclyl and heteroaryl whether an individual entity or as part of a larger entity may be optionally substituted with one or more optional substituents selected from the group consisting of C₁₋₆alkyl, C₂₋₆alkenyl, C₂₋₆alkynyl, C₃₋₆cycloalkyl, oxo(═O), C₁₋₆alkyloxy(CH₂)ₚ—, C₂₋₆alkenyloxy(CH₂)ₚ—, C₂₋₆alkynyloxy(CH₂)ₚ—, C₃₋₆cycloalkoxy(CH₂)ₚ—, C₁₋₆alkylthio(CH₂)ₚ—, C₂₋₆alkenylthio(CH₂)ₚ—, C₂₋₆alkynylthio(CH₂)ₚ—, C₃₋₆cycloalkylthio(CH₂)ₚ—, hydroxy(CH₂)ₚ—, C₁₋₆alkoxyC₁₋₆alkoxy(CH₂)ₚ—, C₁₋₆alkylthioC₁₋₆alkoxy(CH₂)ₚ—, —(CH₂)ₚCO₂H, —(CH₂)ₚCO₂C₁₋₆alkyl,)(CH₂)ₚCON(R¹⁰)₂, C₂₋₆acyl(CH₂)ₚ—, C₂₋₆acyloxy(CH₂)ₚ—, C₂₋₆alkylSO₂(CH₂)ₚ—, C₂₋₆alkenylSO₂(CH₂)ₚ—, C₂₋₆alknylSO₂(CH₂)ₚ—, aryl-SO₂(CH₂)ₚ—, heteroarylSO₂(CH₂)ₚ—, heterocyclylSO₂(CH₂)ₚ—, —(CH₂)ₚNH₂, —CH₂)ₚNH(C₁₋₆alkyl), —(CH₂)ₚN(C₁₋₆alkyl)₂, —(CH₂)ₚNH(phenyl), —(CH₂)ₚN(phenyl)₂, —(CH₂)ₚNH(acyl), —(CH₂)ₚN(acyl)(phenyl), —(CH₂)ₚNH—(CH₂)ₚ—S-aryl, —(CH₂)ₚN═NHC(O)NH₂, —(CH₂)ₚC(R⁴)₃, —(CH₂)ₚOC(R⁴)₃, —(CH₂)ₚSC(R⁴)₃, —(CH₂)ₚCN, —(CH₂)ₚNO₂, —(CH₂)ₚhalogen, —(CH₂)ₚheterocyclyl, heterocyclyloxy(CH₂)ₚ—, —(CH₂)ₚheteroaryl, heteroaryloxy(CH₂)ₚ—, heteroaryloxyalkyloxy(CH₂)ₚ—, heteroaryl-NH-alkoxy(CH₂)ₚ—, heteroaryl-NH-alkyl-NH—(CH₂)ₚ, —(CH₂)ₚaryl, C₂₋₆alkenylaryl, —(CH₂)ₚC(O)aryl, aryloxy(CH₂)ₚ—, aryloxyalkyloxy(CH₂)ₚ— and arylthioalkoxy(CH₂)ₚ—, wherein R⁴ is as defined for formula (I); each R¹⁰ is independently selected from the group consisting of H, C₁₋₆alkyl, phenyl, cycloalkyl or the two R¹⁰ taken together can form a heterocyclyl or heteroaryl ring; and p is 0 or an integer from 1 to 6. Examples of suitable substituents include, but are not limited to, methyl, ethyl, propyl, isopropyl, butyl, sec-butyl, tert-butyl, vinyl, methoxy, ethoxy, propoxy, isopropoxy, butoxy, methylthio, ethylthio, propylthio, isopropylthio, butylthio, hydroxy, hydroxymethyl, hydroxyethyl, hydroxypropyl, hydroxybutyl, fluoro, chloro, bromo, iodo, cyano, nitro, —CO₂H, —CO₂CH₃, —CH₂CO₂CH₃, trifluoromethyl, trifluoromethoxy, trifluoromethylthio, acetyl, morpholino, amino, methylamino, dimethylamino, phenyl, phenylcarbonyl, —NHCOphenyl, —NHCObenzyl, —NHCO(CH₂)₃phenyl or —NHCO(CH₂)₃phenyl in all of which the phenyl ring is optionally substituted with methyl or methoxy, —NHCOethylphenyl, —NHCOCH₂Sphenyl, —N═NHC(O)NH₂, —CH═C(CN)₂ and phenoxy. Particular substituents include fluoro, chloro, methyl, ethyl, propyl, isopropyl, butyl, tent-butyl, methoxy, ethoxy, propoxy, isopropoxy, trifluoromethyl, trifluoromethoxy, cyano, nitro, acetyl, amino, methylamino, dimethylamino, —NHCOphenyl, —NHCObenzyl in which the phenyl ring is optionally substituted with methyl or methoxy, —NHCOethylphenyl, —NHCO(CH₂)₃phenyl and —NHCOCH₂Sphenyl.

The term “isosteric equivalent of a carboxy group” refers to a group which is physiochemically or topologically similar to carboxylic acid or carboxylate group. Examples of suitable carboxylic acid or carboxylate isosteres include, but are not limited to, tetrazole, tetrazolate, —CONH-tetrazole, oxidiazole, phosphate (—PO₃H₂), N-(aryl or heteroaryl)-sulfonamides, acylsulfonamides and sulfonic acid (—SO₃H) [See Patani and LaVoie, 1996]. Examples of isosteric equivalents of carboxy groups include —CONHSO₂R³, —SO₂NHCOR³, —SO₂NHCONHR³, —SO₂NHR^{3a} and —NHSO₂R³, where R³ is selected from the group consisting of C₁₋₇alkyl, C₂₋₇alkenyl, C₂₋₇alkynyl, C₃₋₈cycloalkyl, aryl, heterocyclyl, heteroaryl, —(CH₂)_{q}C₃₋₈cycloalkyl, —(CH₂)_{q}halo, —(CH₂)_{q}OH, —CH—₂)ₜC(R⁴)₃, C₁₋₇alkoxy(CH₂)t—, C₂₋₇alkenyloxy(CH₂)ₜ—, C₂₋₇alkynyloxy(CH₂)ₜ—, C₃₋₈cycloalkyloxy(CH₂)ₜ—, C₁₋₇alkylthio(CH₂)ₜ—, C₂₋₇alkenylthio(CH₂)ₜ—, C₂₋₇alkynylthio(CH₂)ₜ—, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl; and R^{3a} is an aryl or heteroaryl group, where q, r, t and m are as defined above. In a particular embodiment R³ is C₁₋₆alkyl, aryl, trihaloalkyl, C₁₋₇alkylaryl, C₁₋₇alkoxyalkylphenyl, C₁₋₇alkoxyphenyl, C₁₋₇alkylthiophenyl and C₂₋₃alkenylphenyl, in which each alkyl or aryl group may be optionally substituted. Particular optional substituents include —CH₃, phenyl, C₁₋₃alkylphenyl, C₂₋₃alkenylphenyl, halo, NO₂, NH₂ and methoxy. In a particular embodiment R^{3a} is an electron-withdrawing aryl or heteroaryl, in which each aryl or heteroaryl may be optionally substituted, especially phenyl, pyrimidyl and benzothiazolyl.

As used herein, the term “linker” refers to a divalent moiety 1 to 3 atoms in length that links the two groups W and Z. The linker may be a heteroatom such as —O—, —S— or —NR⁵—, or may be an alkylene or alkenylene group in which one or more carbon atoms are optionally replaced with a heteroatom or hetero group selected from the group consisting of N, S(O)ₖ or O wherein k is 0, 1 or 2. Each linker, where appropriate, may be optionally substituted at a carbon or nitrogen atom by one or more optional substituents selected from the group consisting of C₁₋₃alkyl, hydroxy, halogen, C₁₋₃alkoxy, N(R⁵)₂, —C(R⁴)₃, OC(R⁴)₃, NO₂, cyano, SH and C₁₋₃alkylthio. Suitable linkers include, but are not limited to, —NR⁵—, —O—, —S—, —CH₂—, —CH₂—CH₂—, —(CH₂)₃—, —CH(CH₃)—CH₂—, —CH₂—CH(CH₃)—, —CH(CH₃)CH₂CH₂—, —CH₂CH(CH₃)—CH₂—, —CH₂CH₂CH(CH₃)—, —OCH₂—, —CH₂O—, —O(CH₂)₂—, —(CH₂)₂O—, —CH₂OCH₂—, —OCH(CH₃)—, —CH(CH₃)—O—, —CH(CH₃)OCH₂—, —CH₂OCH(CH₃)—, —CH(CH₃)CH₂O—, —OCH₂CH(CH₃)—, —OCH(CH₃)CH₂—, —CH₂CH(CH₃)O—, —S(O)ₖCH₂—, —CH₂S—, —S(O)ₖ(CH₂)₂—, —(CH₂)₂S—, —CH₂S(O)ₖCH₂—, —S(O)ₖCH(CH₃)—, —CH(CH₃)—S—, —CH(CH₃)S(O)ₖCH₂—, —CH₂S(O)ₖCH(CH₃)—, —CH(CH₃)CH₂S—, —S(O)ₖCH₂CH(CH₃)—, —S(O)ₖCH(CH₃)CH₂—, —CH₂CH(CH₃)S—, —NHCH₂—, —CH₂NH—, —NH(CH₂)₂—, —(CH₂)₂NH—, —CH₂NHCH₂—, —NHCH(CH₃)—, —CH(CH₃)—NH—, —CH(CH₃)NHCH₂—, —CH₂NHCH(CH₃)—, —CH(CH₃)CH₂NH—, —NHCH₂CH(CH₃)—, —NHCH(CH₃)CH₂—, —CH₂CH(CH₃)NH—, —CH═CH—, —CH═CH—CH₂—, —CH₂—CH═CH—, —C(CH₃)═CH—, —CH═C(CH₃)—, —CH(CH₃)—CH═CH— and —CH═CH—CH(CH₃)—, where k is 0, 1 or 2.

Each —CH₂— in an alkylene group or each —CH₂— or —CH— in an alkenylene group may be optionally substituted by replacement of one or both hydrogen atoms with an optional substituent as described above. For example, an optionally substituted linker includes, but is not limited to, —CH(OH)—, —CH₂—CH(CF₃)—, —CH(OCH₃)—CH₂—, —O—CH(SCH₃)— and —CH(CH₃)—O—.

The compounds of the invention may be in the form of pharmaceutically acceptable salts. It will be appreciated however that non-pharmaceutically acceptable salts also fall within the scope of the invention since these may be useful as intermediates in the preparation of pharmaceutically acceptable salts or may be useful during storage or transport. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

The compounds and salts of the invention may be presented in the form of a prodrug. The term “prodrug” is used in its broadest sense and encompasses those derivatives that are converted in vivo to the compounds of the invention. Such derivatives would readily occur to those skilled in the art, and include esters and amides (including amino acid esters, amides and conjugates), N-α-acyloxy amides, N-(acyloxyalkoxy carbonyl) amine derivatives and a-acyloxyalkyl esters of phenols and alcohols. A prodrug may include modifications to one or more of the functional groups of a compound of the invention. Examples of ester prodrugs include compounds where Z is CO₂R³⁰ where R³⁰ is selected from the group consisting of C₁₋₇alkyl, C₂₋₇alkenyl, C₂₋₇alkynyl, C₃₋₈cycloalkyl, aryl, heterocyclyl, heteroaryl, —(CH₂)_{q}C₃₋₈cycloalkyl, —(CH₂)_{q}halo, —(CH₂)_{q}OH, C₁₋₇alkoxy(CH₂)_{q}—, C₂₋₇alkenyloxy(CH₂)_{q}—, C₂₋₇alkynyloxy(CH₂)_{q}—, C₃₋₈cycloalkyloxy(CH₂)_{q}—, C₁₋₇alkylthio(CH₂)_{q}—, C₂₋₇alkenylthio(CH₂)_{q}—, C₂₋₇alkynylthio(CH₂)_{q}—, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl, —(CH₂)ᵣM(CH₂)ₜheterocyclyl, —Si(C₁₋₆alkyl)₃ and —(CH₂)ᵣOSi(C₁₋₆alkyl)₃, and where q, r and t are as defined above. In a particular embodiment R³⁰ is C₁₋₇alkyl, C₂₋₇alkenyl, C₂₋₇alkynyl, C₃₋₈cycloalkyl, aryl, heterocyclyl, heteroaryl, (CH₂)_{q}OH, —Si(C₁₋₆alkyl)₃ and —(CH₂)ᵣOSi(C₁₋₆alkyl)₃, especially C₁₋₄alkyl, heterocyclyl, (CH₂)₄OH and —(CH₂)₂OSi(C₁₋₄alkyl)₃.

The term “prodrug” also encompasses the combination of lipids with the compounds of the invention. The presence of lipids may assist in the translocation of the compounds across a cellular membrane and into a cell cytoplasm or nucleus. Suitable lipids include fatty acids which may be linked to the compound by formation of a fatty acid ester. Particular fatty acids include, but are not limited to, lauric acid, caproic acid, palmitic acid and myristic acid.

The phrase “a derivative which is capable of being converted in vivo” as used in relation to another functional group includes all those functional groups or derivatives which upon administration into a mammal may be converted into the stated functional group. Those skilled in the art may readily determine whether a group may be capable of being converted in vivo to another functional group using routine enzymatic or animal studies.

It will also be recognised that compounds of the invention may possess asymmetric centres and are therefore capable of existing in more than one stereoisomeric form. The invention thus also relates to compounds in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution. The compounds of the invention may exist as geometric isomers. The invention also relates to compounds in substantially pure cis (Z) or trans (E) or mixtures thereof.

In particular embodiments, at least one of the following applies:

W is furanyl, thiophenyl, pyrrolyl, N-methylpyrrolyl, pyrazolyl and phenyl, each of which may be optionally substituted. Particular optional substituents include —CH₃, halo, hydroxy, NO₂ and methoxy;

L is a linker selected from the group consisting of —NH—, —CH₂—, —CH₂CH₂—, —OCH₂—, —SCH₂—, —NHCH₂—, —CH₂NH—, —O—CH(CH₃)— and —CH═CH—;

Q is an aryl or heteroaryl group selected from the group consisting of phenyl, pyridyl, furanyl, thiophenyl, pyrazolyl, pyrrolyl, N-methylpyrrolyl, thiazole, oxazole, triazole and pyrimidyl, each of which may be optionally substituted. Particular optional substituents include —CH₃, hydroxy, OCOCH₃, OCH₂CH₂CH₂OC₆H₅, halo, NO₂, CO₂H, NH₃, NHCOC₆H₅, NHCOCH₃, NHCOCH₂C₆H₄(4-OMe), NHCOCH₂C₆H₅, NHCO(CH₂)₂C₆H₄(4-OMe), NHCO(CH₂)₂C₆H₅, NHCOCH₂SC₆H₅, NHCO(CH₂)₂C₆H₅ and methoxy;

Z is CO₂H, SO₂NHCOR³ and CONHSO₂R³. In some embodiments, Z is CO₂H;

when W—X is W-Q-Y—Z, the attachment of Q to W is in a 1,3-arrangement with the attachment of W to the benzothiazole or benzoselenazole hydrazone moiety;

when W—X is —W-Q-Y—Z, W and Y are positioned in a 1,3-arrangement with respect to Q;

when W—X is W-L-Z, the attachment of L-Z to W is in a 1,4-arrangement with the attachment of W to the benzothiazole or benzoselenazole hydrazone moiety;

n is 0;

however, when n is 1 or 2, in particular embodiments, R¹ is halo, methoxy, CF₃O and methyl, especially fluoro; and

R² is C₁₋₆alkyl, especially C₁₋₄alkyl; or R² together with a carbon atom in the aryl or heteroaryl ring of W forms an optionally substituted 5 to 8 membered carbocyclic or heterocyclic ring, especially a 5, 6 or 7 membered carbocyclic or heterocyclic ring. In a particular embodiment the 5 to 8 membered carbocyclic or heterocyclic ring is unsubstituted. In some embodiments, R² and the carbon atom of W, together with atoms to which they are attached form an optionally substituted indanyl group, a tetrahydronaphthylene group, a chromanyl group, a tetrahydroquinolinyl or N-alkyl-tetrahydroquinolinyl group, a benzothiopyranyl group, a benzocycloheptenyl group, an S-oxido or S-dioxido-benzothiopyranyl group, a dihydroindolyl group or a 2-oxodihydroindolyl group. Particular optional substituents include C₁₋₃alkyl, halo, hydroxy, NO₂, C₁₋₃alkoxy, trifluoromethyl and oxo (═O), especially methyl, halo, hydroxy, NO₂, methoxy, trifluoromethoxy and oxo.

R³ is C₁₋₆alkyl, aryl, C₁₋₇alkylaryl, C₁₋₇alkoxyalkylphenyl, C₁₋₇alkoxyphenyl, C₁₋₇alkylthiophenyl and C₂₋₃alkenylphenyl, in which each alkyl or aryl group may be optionally substituted. Particular optional substituents include —CH₃, phenyl, halo, NO₂, NH₂ and methoxy.

Particular prodrugs are ester prodrugs, particularly esters where Z is CO₂R³⁰ and R³⁰ is —C₁₋₆alkylhydroxy, —C₁₋₆alkylheterocyclyl and —C₁₋₆alkylOSi(C₁₋₆alkyl)₃, especially where R³⁰ is ethyl, —CH₂CH₂OH, CH₂CH₂N-morpholino and —CH₂CH_{2OSi(C}₁₋₆alkyl)₃.

In some embodiments, the compound of the invention is a compound of formula (II):

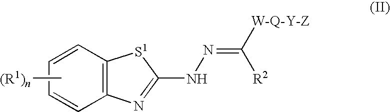

where S¹, W, Q, Y, Z, R¹, R² and n are as defined for formula (I), or salts, esters or isomers thereof.

A particular compound of formula (II) is a compound of formula (IIa):

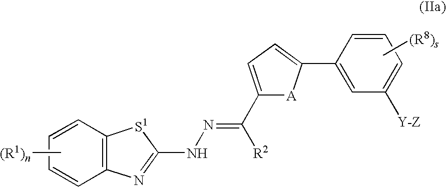

wherein

A is —O—, —S— or N(R⁵);

R⁸ is C₁₋₄alkyl, C₂₋₄alkenyl, C₂₋₄alkynyl, hydroxy, C₁₋₄alkoxy, C₁₋₄alkenyloxy, C₁₋₄alkynyloxy, C₂₋₄acyloxy, aryloxyC₁₋₄alkyloxy, halogen, —C(R⁴)₃, nitro, cyano, N(R⁵)₂, NHCOC₁₋₄alkyl, NHCOaryl, NHCOOC₁₋₄alkyl and NHCOC₁₋₄alkylaryl wherein each aryl group may be optionally substituted with methyl or methoxy;

s is 0, 1 or 2;

S¹, Y, Z, R¹, R², R⁴, R⁵, R⁶ and n are as defined for formula (I), or salts, esters or isomers thereof.

Another particular compound of formula (II) is a compound of formula (IIb):

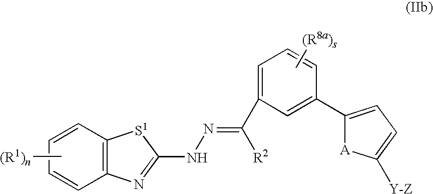

wherein S¹, Y, Z, A, R¹, R², R⁴, R⁵, n and s are as defined for formula (IIa); and

R^{8a} is C₁₋₄alkyl, C₂₋₄alkenyl, C₂₋₄alkynyl, hydroxy, C₁₋₄alkoxy, C₁₋₄alkenyloxy, C₁₋₄alkynyloxy, halogen, —C(R⁴)₃, nitro, cyano and N(R⁵)₂;

or salts, esters or isomers thereof.

Another particular compound of formula (II) is a compound of formula (IIc):

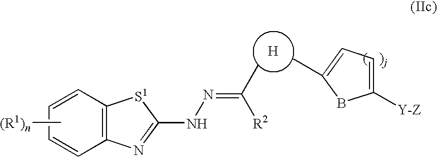

wherein S¹, Y, Z, R¹, R², R⁵ and n are as defined in formula (I);

H is a 5 or 6 membered heteroaryl group, wherein the benzothiazole hydrazone moiety and the aryl or heteroaryl group bearing the group Y-Z are bonded to group H in a 1,3 arrangement;

B is —O—, S, or —N(R⁵)— when j is 1, or

B is —N— or —CH— when j is 2, or salts, esters or isomers thereof.

Another particular compound of formula (II) is a compound of formula (IId):

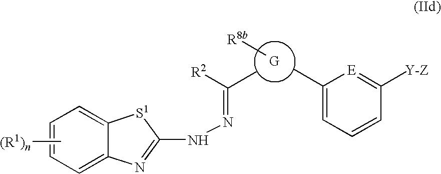

wherein S¹, Y, Z, R¹ and R² are as defined in formula (I);

G is a phenyl group or a 5 or 6 membered heteroaryl group, wherein the benzothiazole hydrazone moiety and the aryl or heteroaryl group bearing the group Y—Z are bonded to group G in a 1,3 arrangement;

E is —N— or —CH—;

R^{8b} is H or is R⁸ as defined in formula (IIa), or

R^{8b} and R² taken together form a 5 to 8 membered carbocyclic or heterocyclic ring, and salts, esters or isomers thereof.

Another particular embodiment of formula (II) is a compound of formula (IIe):

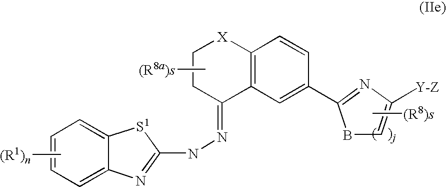

wherein S¹, Y, Z, R¹, R⁵ and n are as defined in formula (I);

X is —CH₂—, —CH₂CH₂— or —O—;

B is —O—, —S— or —NR⁵— when j is 1, or

B is —N— or —CH— when j is 2; and

R⁸ and s are as defined for formula (IIa),

R^{8a} is as defined in formula (IIb) or is oxo (═O),

or salts, esters or isomers thereof.

In some embodiments, the compound of the invention is a compound of formula (III):

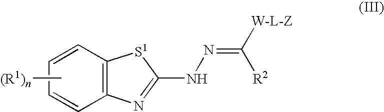

wherein S¹, W, L, Z, R¹ , R² and n are as defined for formula (I) or salts, esters or isomers thereof, provided that said compound is other than [4-[1-[(6-ethoxy-2-benzothiazolyl)hydrazono]ethyl]phenoxy]-acetic acid; [4-[1-[(6-methoxy-2-benzothiazolyl)hydrazono]ethyl]phenoxy]-acetic acid; [4-[1-[(6-methyl-2-benzothiazolyl)hydrazono]ethyl]phenoxy]-acetic acid; [4-[1-[(6-chloro-2-benzothiazolyl)hydrazono]ethyl]phenoxy]-acetic acid; and [4-[1-[(2-benzothiazolylhydrazono)ethyl]phenoxy]-acetic acid.

A particular compound of formula (III) is a compound of formula (IIIa):

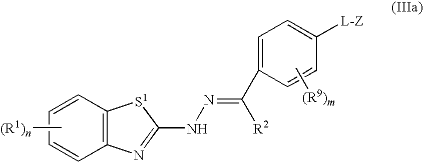

wherein S¹, L, Z, R¹, R² and n are as defined in formula (I); and

R⁹ is C₁₋₄alkyl, C₂₋₄alkenyl, C₂₋₄alkynyl, hydroxy, C₁₋₄alkoxy, C₁₋₄alkenyloxy, C₁₋₄alkynyloxy, halogen, —C(R⁴)₃, nitro, cyano and —N(R⁵)₂;

or when R⁹ and R² taken together form a 5 to 8 membered carbocyclic or heterocyclic ring, and m is 0, 1 or 2 or salts, esters or isomers thereof, provided that said compound is other than [4-[1-[(6-ethoxy-2-benzothiazolyl)hydrazono]ethyl]phenoxy]-acetic acid; [4-[1-[(6-methoxy-2-benzothiazolyl)hydrazono]ethyl]phenoxy]-acetic acid; [4-[1-[(6-methyl-2-benzothiazolyl)hydrazono]ethyl]phenoxy]-acetic acid; [4-[1-[(6-chloro-2-benzothiazolyl)hydrazono]ethyl]phenoxy]-acetic acid; and [4-[1-(2-benzothiazolylhydrazono)ethyl]phenoxy]-acetic acid.

Particular compounds of the invention are set out in Tables 1-11 below:

TABLE 1

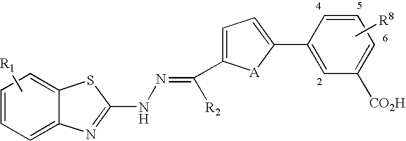

| Compound No. | R₁ | R₂ | A | R⁸ |
|---|---|---|---|---|
| 1 | H | CH₃ | O | 2-CH₃ |
| 2 | H | CH₃ | O | H |
| 3 | H | CH₃ | O | 4-Cl |
| 4 | H | CH₃ | O | 6-Cl |
| 5 | H | CH₃ | O | 6-NO₂ |
| 6 | H | CH₂CH₃ | O | H |
| 7 | H | CH₃ | S | 6-NO₂ |
| 8 | H | CH₃ | S | 6-Cl |
| 9 | H | CH₂CH(CH₃₎₂ | O | 6-Cl |
| 10 | H | CH(CH₃₎₂ | O | 6-Cl |
| 11 | H | CH₂CH₃ | O | 6-Cl |
| 12 | H | CH₃ | NMe | 6-Cl |
| 13 | H | CH₃ | NH | 6-Cl |
| 14 | H | CH₂CH₂CH₂CH₃ | O | 6-Cl |
| 15 | H | CH₃ | O | 5-CO₂H |
| 16 | H | CH₃ | O | 6-NH₂ |
| 17 | H | CH₃ | O | 6-NHCOC₆H₅ |
| 18 | H | CH₃ | O | 6-NHCOCH₃ |
| 19 | H | CH₃ | O | 6-NHCOCH₂C₆H₄(4-OMe) |
| 20 | 6-F | CH₃ | O | 4-Cl |
| 21 | 6-F | CH₃ | O | 2-CH₃ |
| 22 | H | CH₃ | O | 2-Cl |
| 23 | H | CH₃ | O | 6-CO₂H |
| 24 | H | CH₃ | O | 6-NHCOCH₂C6H5 |
| 25 | H | CH₃ | O | 6-NHCO(CH₂)₂C₆H₄(4-OMe) |
| 26 | 6-Br | CH₃ | O | 6-Cl |
| 27 | 6-Cl | CH₃ | O | 6-Cl |
| 28 | 6-CF₃ | CH₃ | O | 6-Cl |
| 29 | 5-Cl, 6-F | CH₃ | O | 6-Cl |
| 30 | 5-Br | CH₃ | O | 6-Cl |
| 31 | 4-Br, 6-CF₃O | CH₃ | O | 6-Cl |
| 32 | 6-MeO | CH₃ | O | 6-Cl |
| 33 | 4-F | CH₃ | O | 6-Cl |
| 34 | 4,6-Cl₂ | CH₃ | O | 6-Cl |
| 35 | 4,6-Me₂ | CH₃ | O | 6-Cl |
| 36 | H | CH₃ | O | 6-NHCO(CH₂)₂C₆H₅ |
| 37 | H | CH₃ | O | 6-NHCOCH₂SC₆H₅ |
| 38 | H | CH₃ | O | 6-NHCO(CH₂)₃C₆H₅ |
| 39 | H | CH₃ | S | 2-CH₃ |
| 40 | H | CH₃ | O | 6-OH |
| 41 | H | CH₂OH | O | H |

TABLE 2

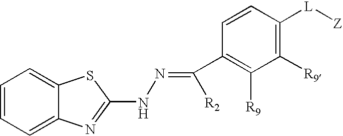

| Compound No. | R₂ | R₉ | R_{9′} | L | Z |
|---|---|---|---|---|---|
| 200 | CH₃ | H | H | OCH₂ | CO₂H |
| 201 | CH₃ | H | H | NHCH₂ | CO₂H |
| 202 | CH₂CH₂CH₂ | | H | OCH₂ | CO₂H |
| 203 | CH₃ | H | H | CH═CH | CO₂H |
| 204 | CH₂CH₂CH₂ | | H | OCH(CH₃) | CO₂H |
| 205 | CH₂CH₂ | | H | OCH₂ | CO₂H |
| 206 | CH₃ | H | NO₂ | NHCH₂ | CO₂H |
| 207 | (CH₂)₃CH₃ | H | H | OCH₂ | CO₂H |
| 208 | CH₂CH₂CH₂ | | H | OCH₂ | CONHSO₂Me |
| 209 | CH₂CH₂CH₂ | | H | OCH₂ | CONHSO₂CH₂C₆H₅ |
| 210 | CH₃ | H | H | SCH₂ | CO₂H |
| 211 | CH₃ | H | H | SO₂CH₂ | CO₂H |
| 212 | CH₃ | H | H | NHCO | CO₂H |

TABLE 3

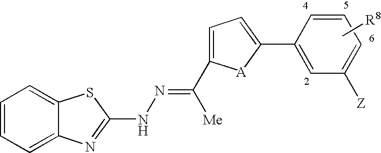

| Compound | | | |
|---|---|---|---|
| No. | A | R⁸ | Z |
| 300 | O | H | SO₂NHCOCH₃ |
| 301 | NMe | 6-Cl | CONHSO₂CH₃ |
| 302 | O | H | CH₂CO₂H |
| 303 | NMe | 6-Cl | CONHSO₂C₆H₄(4-Br) |
| 304 | O | H | SO₂NHCO(CH₂)₂C₆H₅ |
| 305 | O | H | SO₂NHC0(CH₂)₃C₆H₅ |
| 306 | S | H | SO₂NHCOCH₃ |
| 307 | O | H | SO₂NHCOC₆H₅ |
| 308 | O | H | SO₂NHCOCH₂C₆H₅ |
| 309 | O | H | SO₂NHCOCH(C₆H₅)₂ |
| 310 | S | 6-Cl | CONHSO₂CH₃ |
| 311 | O | 6-Cl | CONHSO₂CH₃ |
| 312 | O | H | SO₂NHCOC₆H₄-(3-C₆H₅₎ |
| 313 | O | H | SO₂NHCOC₆H₄-(4-C₆H₅₎ |
| 314 | O | 6-Cl | CONHSO₂CH₂C₆H₅ |
| 315 | O | 6-Cl | CONHSO₂(CH₂)₂C₆H₅ |
| 316 | O | 6-Cl | CONHSO₂(CH₂)₃C₆H₅ |
| 317 | O | H | SO₂NHCOCH₂OCH₂C₆H₅ |
| 318 | O | H | SO₂NHCO(CH₂)₂OC₆H₅ |
| 319 | O | H | SO₂NHCO(CH₂)₂SC₆H₅ |
| 320 | O | H | SO₂NHCO3CH(NH₂)(CH₂)₂C₆H₅ |
| | | | (enantiomer) |
| 321 | O | H | SO₂NHCO(CH₂)₄C₆H₅ |
| 322 | O | H | SO₂NHCO(CH₂)₅C₆H₅ |
| 323 | O | H | SO₂NHCOCH═CHCH₂C₆H₅ |
| 324 | O | H | SO₂NHCO(CH₂)₃C₆H₄-(4-OMe) |
| 325 | O | H | SO₂NHCO(CH₂)₃C₆H₄-(4-I) |
| 326 | O | H | SO₂NHCO(CH₂)₃C₆H₃-[3,4-(OMe)₂] |
| 327 | O | H | SO₂NHCO(CH₂)₃C₆H₃-[2,5-(OMe)₂] |
| 328 | O | H | SO₂NHCO(CH₂)₃C₆H₄-(4-NO₂) |
| 329 | O | H | SO₂NHCOCH₂CH(NBoc)CH₂C₆H₅ |
| | | | (enantiomer) |
| 330 | O | H | SO₂NHCOCH₂CH(NBoc)CH₂C₆H₅ |
| | | | (enantiomer) |
| 331 | O | H | SO₂NHCOCH₂CH(NH₂)CH₂C₆H₅ |
| | | | (enantiomer) |
| 332 | S | H | CH₂SO₂NHCOCH₃ |
| 333 | S | H | CH₂SO₂NHCOC₆H₅ |
| 334 | S | H | CH₂SO₂NHCOCH₂C₆H₅ |
| 335 | S | H | CH₂SO₂NHCO(CH₂)₂C₆H₅ |
| 336 | S | H | CH₂SO₂NHCO(CH₂)₃C₆H₅ |
| 337 | O | H | CONHSO₂CH₃ |
| 338 | O | H | CONHSO₂CH₂C₆H₅ |
| 339 | O | H | SO₂NHCOCH₂CH(NH₂)CH₂C₆H₅ |
| | | | (enantiomer) |
| 340 | O | H | SO₂NHCOCH(NH₂)CH₂SC₆H₅ |
| 341 | O | H | SO₂NHCO(CH₂)₃C₆H₄-(4-Me) |
| 342 | O | H | SO₂NHCO(CH₂)₂CH₃ |
| 343 | O | H | SO₂NHCO(CH₂)₃C₆H₄-(3-Phenyl) |
| 344 | O | H | SO₂NHCOCH(NH₂)(CH₂)₂C₆H₅ |
| 345 | O | H | SO₂NHCOCH₂CH(NH₂)CH₃ |
| 346 | O | H | SO₂NHCO(CH₂)₃NH₂ |
| 347 | S | H | CONHSO₂Me |
| 348 | S | H | CONHSO₂CH₂C₆H₅ |
| 349 | O | 6-Cl | CONHSO₂(CH₂)₄C₆H₅ |
| 350 | O | H | CONHSO₂(CH₂)₄C₆H₅ |
| 351 | O | H | SO₂NHCO(CH₂)₃C₆H₄-(4-Phenyl) |
| 352 | O | H | SO₂NHCO(CH₂)₃C₆H₄-(3-Br) |
| 353 | O | H | SO₂NHCO(CH₂)₃C₆H₃-(3,4-OCH₂CH₂O) |
| 354 | S | H | SO₂NHCO(CH₂)₂SC₆H₅ |
| 355 | O | 4-Cl | SO₂NHCOCH₃ |
| 356 | O | 4-Me | SO₂NHCOCH₃ |
| 357 | O | 4-Me | SO₂NHCO(CH₂)₂SC₆H₅ |
| 358 | O | 6-Cl | SO₂NHCOCH₃ |
| 359 | O | 6-Cl | SO₂NHCO(CH₂)₂SC₆H₅ |
| 36O | O | H | SO₂NHCOCH(NHBoc)CH₂SCH₂CH₂C₆H₅ |
| 361 | O | H | SO₂NHCOCH(NH₂)CH₂SCH₂CH₂C₆H₅ |
| 362 | O | H | SO₂NHCOCH₂CH₂SCH₂CH(Me)₂ |
| 363 | O | H | SO₂NHCOCH₂CH₂SCH₂CH₂CH₃ |
| 364 | O | H | SO₂NHCOCH₂CH₂SCH(Me)₂ |
| 365 | O | H | SO₂NHCOCH₂CH₂SCH₂CH₃ |
| 366 | O | H | SO₂NHCOCH₂CH₂CH₂CH₃ |
| 367 | O | H | SO₂NHCOCH₂CH₂CH(Me)₂ |
| 368 | O | H | SO₂NHCOCH₂CH(Me)₂ |
| 369 | O | H | SO₂NHCO(CH₂)₂SC₆H₄-(4-OMe) |
| 370 | O | H | SO₂NHCOCH(NH₂)CH₂SCH(Me)₂ |
| 371 | O | H | SO₂NHCO(CH₂)₂NH₂ |
| 372 | O | H | SO₂NHCOCH(NH₂)CH₂-1-Naphthyl |
| 373 | O | H | SO₂NHCOCH(NH₂)CH₂SCH₂C₆H₅ |
| 374 | O | H | SO₂NHCOCH(NH₂)CH₂CH₂SCH₂C₆H₅ |
| 375 | O | H | SO₂NHCO(CH₂)₃C₆H₄-(2-Phenyl) |
| 376 | O | H | SO₂NHCOC₆H₄-[3-(CH₂)₂C₆H_{5]} |
| 377 | O | H | SO₂NHCOC₆H₄-(3-CH═CH—C₆H₅₎ |
| 378 | O | H | SO₂NHCOC₆H₄-[3-CH₂C₆H_{5]} |
| 379 | O | H | SO₂NHCOC₆H₄-[4-CH₂C₆H_{5]} |
| 380 | O | H | SO₂NHCOCH(NH₂)CH₂C₆H₅ |
| 381 | O | H | SO₂NHCOCH(NH₂)CH₂OCH₂C₆H₅ |
| 382 | O | H | SO₂NHCO(CH₂)₂SC₆H₄-(4-F) |
| 383 | O | H | SO₂NHCOCH(NH₂)CH₂SCH₂CH(Me)₂ |
| 384 | O | H | SO₂NHCOCH(NH₂)CH₂SCH₂CH₂CH₃ |
| 385 | O | H | SO₂NHCOCH(NH₂)CH₂SCH₂CH₃ |
| 386 | O | H | SO₂NHCOCH(NH₂)CH₃ |
| 387 | O | H | SO₃H |
| 388 | O | H | SO₂NH—C₆H₃-(2-NO₂-4-CF₃) |
| 389 | O | H | SO₂NH-2-pyrimidyl |
| 390 | O | H | SO₂NH-2-benzothiazolyl |

TABLE 4

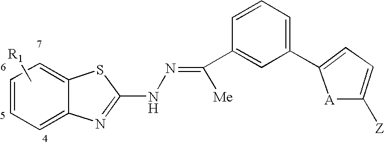

| Compound No. | R₁ | A | Z |
|---|---|---|---|
| 400 | H | O | CO₂H |
| 401 | H | S | CO₂H |

TABLE 5

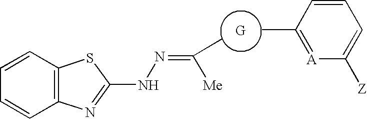

| Compound No. | G | Group A | Z |
|---|---|---|---|
| 500 | 1,3-phenyl | CH | CO₂H |
| 501 | 3-methyl-5-hydroxy- | CH | CO₂H |
| | 1,4-pyrazolyl | | |
| 502 | 2,5-thienyl | N | CO₂H |
| 503 | 1,3-phenyl | CH | SO₂NHCOCH₃ |

TABLE 6

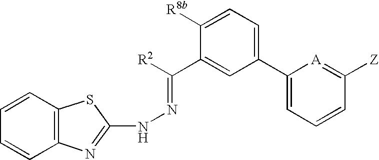

| Compound No. | Group R² | Group R^{8b} | Group A | Z |
|---|---|---|---|---|
| 600 | CH₃ | H | N | CO₂H |
| 601 | CH₃ | H | N | CONHSO₂(CH₂)₃C₆H₅ |
| 602 | CH₃ | H | N | CO₂Et |
| 603 | CH₃ | H | N | CO₂CH₂CH₂N(CH₂CH₂)₂O |
| 604 | CH₃ | H | N | CO₂CH₂CH₂OSi(Me)₂Bu^{t} |
| 605 | CH₃ | H | N | CO₂CH₂CH₂OH |
| 606 | CH₂CH₂ | | CH | CO₂H |
| 607 | CH₃ | H | N | Tetrazol-5-yl |
| 608 | CH₃ | H | N | CONHSO₂Me |
| 609 | CH₃ | H | N | CONH-(tetrazol-5-yl) |
| 610 | CH₂CH₂ | | N | CO₂H |
| 611 | CH₂CH₂CH₂ | | N | CO₂H |
| 612 | CH₂CH₂O | | N | CO₂H |
| 613 | CH₃ | H | N | SO₂NHCOCH₃ |
| 614 | CH₂CH₂CH₂ | | CH | CO₂H |
| 615 | CH₂CH₂CH₂ | | N | SO₂NHCOCH₃ |
| 616 | CH₂CH₂CH₂ | | N | SO₂NHCO(CH₂)₅C₆H₅ |
| 617 | CH₂CH₂CH₂ | | N | CONHSO₂(CH₂)₅C₆H₅ |
| 618 | CH₂CH₂CH₂ | | N | NHSO₂C₆H₄-(4-Me) |
| 619 | CH₂CH₂CH₂ | | N | NHSO₂Me |
| 620 | CH₃ | H | N | NHSO₂Me |
| 621 | CH₂CH₂CH₂ | | N | NHSO₂CF₃ |
| 622 | CH₃ | H | N | NHSO₂C₆H₄-(4-Me) |
| 623 | CH₃ | H | N | NHSO₂CF₃ |

| | | | |
|---|---|---|---|
| 624 | CH₂CH₂CH₂ | N | CO₂Et |
| 625 | CH₂CH₂N(Et) | N | CO₂H |
| 626 | CH₂CH₂NH | N | CO₂H |
| 627 | CH₂CH₂S | N | CO₂H |
| 628 | CH₂CH₂CH₂CH₂ | N | CO₂H |
| 629 | CH₂CH₂CH₂ | N | CO₂CH₂CH₂N(CH₂CH₂)₂O |
| 630 | CH₂CH₂S(O) | N | CO₂H |
| 631 | CH₂CH₂S(O)₂ | N | CO₂H |
| 632 | CH₂CH₂S | N | CO₂CH₂CH₂N(CH₂CH₂)₂O |
| 633 | CH₂CH(CF₃)NH | N | CO₂H |
| 634 | CH₂C(Me)₂O | N | CO₂H |
| 635 | CH(Me)CH₂CH₂ | N | CO₂H |
| 636 | C(Me)₂CH₂CH₂ | N | CO₂H |
| 637 | CH₂CH(Me)CH₂ | N | CO₂H |
| 638 | CH₂CH₂CH₂O | N | CO₂H |
| 639 | C(═O)NMe | N | CO₂H |

TABLE 7

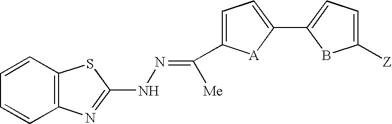

| Compound No. | A | B | Z |
|---|---|---|---|
| 700 | S | O | CO₂H |
| 701 | S | S | CO₂H |

TABLE 8

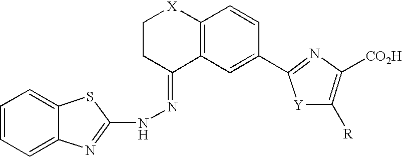

| Compound No. | Group X | Group Y | R substituent |
|---|---|---|---|
| 800 | CH₂ | S | H |
| 801 | CH₂ | O | H |
| 802 | O | S | H |
| 803 | CH₂CH₂ | S | H |
| 804 | CH₂ | N═CH | H |

TABLE 9

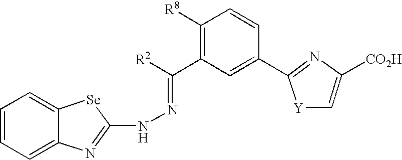

| Compound No. | Group R² | Group R⁸ | Group Y |
|---|---|---|---|
| 900 | Me | H | CH═CH |

| | | |
|---|---|---|
| 901 | CH₂CH₂CH₂ | CH═CH |
| 902 | CH₂CH₂S | CH═CH |

TABLE 10

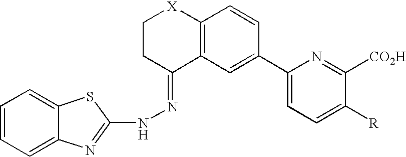

| Compound No. | Group X | Substituent R |
|---|---|---|
| 1000 | CH₂ | OH |
| 1001 | CH₂ | OCOCH₃ |
| 1002 | CH₂ | OCH₂CH₂CH₂OC₆H₅ |
| 1003 | CH₂ | OCH₃ |

TABLE 11
Miscellaneous compounds

| Compound No. | Structure |
|---|---|
| 1100 | 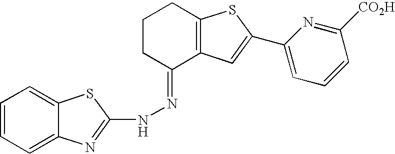 |
| 1101 | 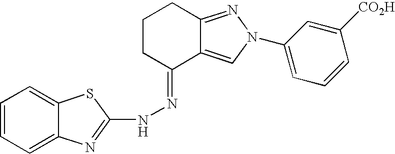 |

In particular embodiments, the compound of formula (I) is selected from the group consisting of compounds 1, 2, 4-9, 11-14, 16, 18-22, 24-26, 33, 36, 39-40, 200, 202, 206-208, 300, 302-309, 311-314, 316-331, 335-349, 358, 360-378, 380-389, 400, 500, 502, 503, 600, 601, 606-612, 615, 624, 627-631, 633, 634, 637, 638, 700, 701, 800, 802, 803, 900, 901, 902 and 1002. In another particular embodiment the compounds of formula (I) are selected from the group consisting of 600, 610, 611, 612, 615, 624, 627, 628, 629, 630, 631, 633, 634, 637, 638, 800, 802, 803 and 1002.

The compounds of the invention may be prepared by reacting a suitably substituted 2-hydrazino-benzothiazole or benzoselenazole compound with a group R²—(C═O)—W—X by heating in an appropriate solvent, such as a polar solvent, eg. ethanol or acetic acid (Scheme 1).

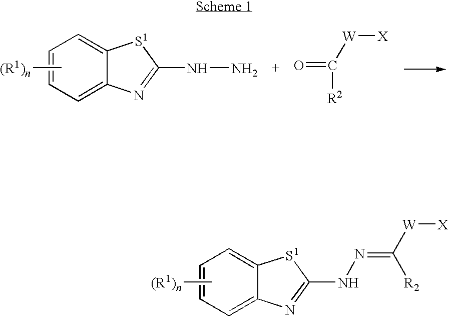

2-Hydrazinobenzothiazole compounds may be prepared from commercially available 2-chlorobenzothiazole compounds as shown in Scheme 2. 2-Hydrazino-benzoselenazole compounds were prepared by the method of Reynolds, 1959.

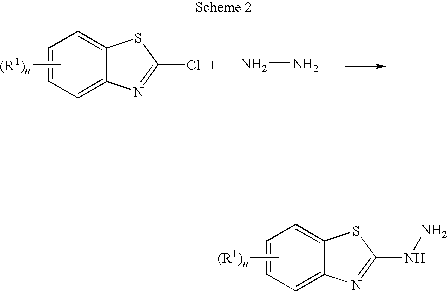

Compounds of formula (II) may be prepared from a suitably substituted 2-hydrazino-benzothiazole or benzoselenazole compound and a group R²—(C═O)—W-Q-Y—Z as shown in Scheme 1. The group R²—(C═O)—W-Q-Y—Z may be prepared using Meerwein arylation reaction as shown in Scheme 3.

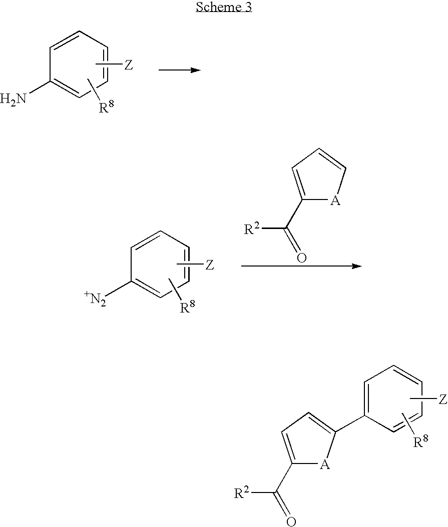

It will be clear to a person skilled in the art that a similar reaction starting from, for example, a 3-acyl-1-aminobenzene and a carboxy substituted heterocyclic group, will allow the positions of the aryl and heteroaryl groups to be rearranged as shown in Scheme 4.

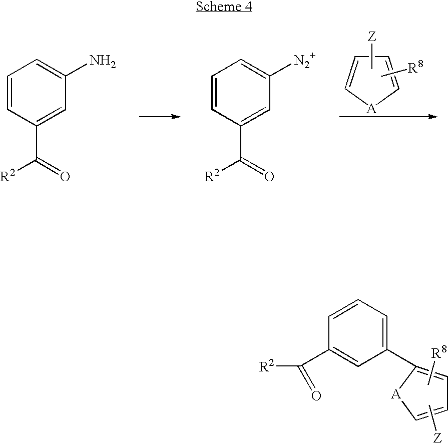

Similarly compounds in which both the groups W and Q are one of aryl or heteroaryl can be prepared in the same manner using the Meerwein arylation reaction.

An alternative method for reacting two aryl and/or heteroaryl groups together is a Suzuki coupling where an aryl- or heteroaryl-borane is coupled with an aryl- or heteroaryl-halide in the presence of a palladium catalyst as shown in Schemes 5 and 6.

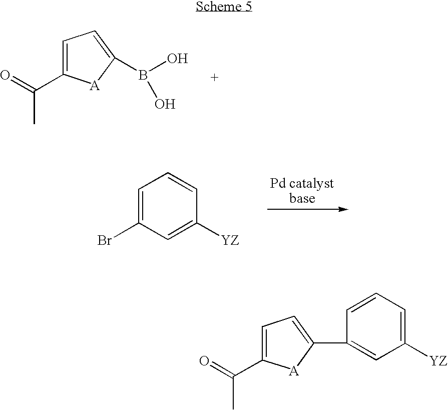

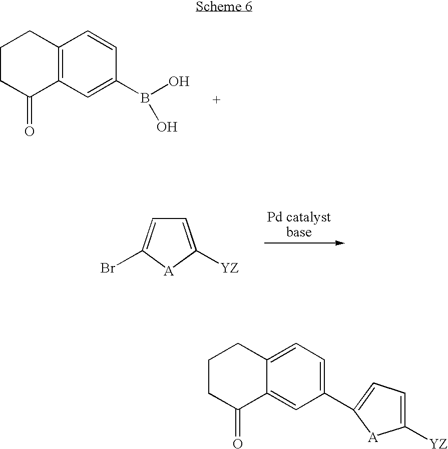

Compounds of formula (III) may be prepared from a suitably substituted 2-hydrazino-benzothiazole or benzoselenazole compound and a group R²(C═O)—W-L-Z as shown in

Scheme 1. Many compounds of the formula R²(C=O)—W-L-Z are commercially available, others may be prepared using methods known in the art.

A person skilled in the art will be aware that during synthesis of the compounds of the invention, some substituents may be reactive under conditions used and must be disguised or protected to prevent unwanted side reactions. Suitable protecting groups for a protecting reactive groups from unwanted reactions are provided in Green and Wuts, Protective Groups in Organic Synthesis. In the alternative, groups may be disguised. For example, if R⁷ is an amino group, this may interfere with the Meerwein arylation reaction and therefore may initially be present as a nitro (NO₂) group. After arylation, the nitro group may be reduced to provide an amino group.

Substituents present on the benzothiazole moiety (R¹), the aryl moieties (R⁷, R⁸, L, Z) or the substituent R² may be manipulated or introduced before or after condensation of the hydrazinobenzothiazole and ketone as shown in Scheme 1. For example, initially Z may be CO₂H or SO₃H but further reaction with a sulfonamide or amide respectively will allow conversion of carboxylic or sulfonic acid to an acylsulfonamide.

In another aspect of the invention there is provided a method of regulating the death of a cell comprising contacting the cell with an effective amount of the formula (I):

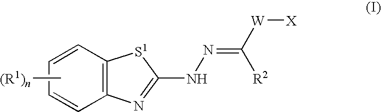

wherein

S¹ is S or Se;

W is an aryl or heteroaryl group;

X is -L-Z or -Q-Y—Z;

L is a linker of 1 to 3 atoms in length;

Q is an aryl or heteroaryl group;

Y is absent or is C₁₋₂alkylene;

Z is a carboxy group or an optionally substituted isosteric equivalent of a carboxy group;

R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyan and —N(R⁵)₂;

R² is selected from the group consisting of C₁₋₇alkyl, C₂₋₇alkenyl, C₂₋₇alkynyl, —(CH₂)_{q}C₃₋₈cycloalkyl, —(CH₂)_{q}halo, —(CH₂)_{q}OH, C₁₋₇alkoxy(CH₂)ₜ—, C₂₋₇alkenyloxy(CH₂)ₜ—, C₂₋₇alkynyloxy(CH₂)ₜ—, C₃₋₈cycloalkyloxy(CH₂)ₜ—, C₁₋₇alkylthio(CH₂)ₜ—, C₂₋₇alkenylthio(CH₂)ₜ—, C₂₋₇alkynylthio(CH₂)ₜ—, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{COR}⁷, —(CH₂)ᵣM(CH₂)ᵣhalo, —(CH₂)ᵣM(CH₂)ᵣOH, —(CH₂)ₜM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂),M(CH₂)ₜheterocyclyl,

or R² together with a ring atom from W forms an optionally substituted 5-8 membered carbocyclic or heterocyclic ring;

each R⁴ is independently selected from the group consisting of hydrogen and halogen;

each R⁵ is independently selected from the group consisting of hydrogen and C₁₋₃alkyl;

each R⁶ is independently selected from the group consisting of hydrogen, C₁₋₇alkyl, C₂₋₇alkenyl, C₂₋₇alkynyl, C₃₋₈cycloalkyl, acyl, aryl, heterocyclyl or heteroaryl, or two R⁶ taken together with the nitrogen to which they are attached form an optionally substituted carbocyclic or heterocyclic ring;

R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃₋₈cycloalkyloxy, aryloxy, heteroaryloxy, heterocyclyloxy and N(R⁶)₂;

M is O, S or NR⁵;

n is 0, 1 or 2;

q is 1 to 7;

r is 1 to 4;

t is 0 or 1 to 4;

wherein each alkyl, alkenyl, alkynyl, alkylene, linker, carbocyclic, heterocyclic, aryl and heteroaryl group may be optionally substituted with one or more optional substituents, and salts, esters and isomers thereof.

In another aspect of the invention there is provided a method of inducing apoptosis in unwanted or damaged cells comprising contacting said damaged or unwanted cells with an effective amount of a compound of formula (I):

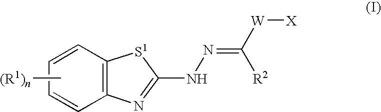

wherein

S¹ is S or Se;

W is an aryl or heteroaryl group;

X is -L-Z or -Q-Y—Z;

L is a linker of 1 to 3 atoms in length;

Q is an aryl or heteroaryl group;

Y is absent or is C₁₋₂alkylene;

Z is a carboxy group or an optionally substituted isosteric equivalent of a carboxy group;

R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyano and —N(R⁵)₂;

R² is selected from the group consisting of C₁₋₇alkyl, C₂₋₇alkenyl, C₂₋₇alkynyl, —(CH₂)_{q}C₃₋₈cycloalkyl, —(CH₂)_{q}halo, —(CH₂)_{q}OH, C₁₋₇alkoxy(CH₂)ₜ—, C₂₋₇alkenyloxy(CH₂)ₜ—, C₂₋₇alkynyloxy(CH₂)ₜ—, C₃₋₈cycloalkyloxy(CH₂)ₜ—, C₁₋₇alkylthio(CH₂)ₜ—, C₂₋₇alkenythio(CH₂)ₜ—, C₂₋₇alkynylthio(CH₂)ₜ—, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl,

or R² together with a ring atom from W forms an optionally substituted 5-8 membered carbocyclic or heterocyclic ring;

each R⁴ is independently selected from the group consisting of hydrogen and halogen;

each R⁵ is independently selected from the group consisting of hydrogen and C₁₋₃alkyl;

each R⁶ is independently selected from the group consisting of hydrogen, C₁₋₇alkyl, C₂₋₇alkenyl, C₂₋₇alkynyl, C₃₋₈cycloalkyl, acyl, aryl, heterocyclyl or heteroaryl, or two R⁶ taken together with the nitrogen to which they are attached form an optionally substituted carbocyclic or heterocyclic ring;

R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃_{_{—}}₈cycloalkyloxy, aryloxy, heteroaryloxy, heterocyclyloxy and N(R⁶)₂;

M is O, S or NR⁵;

n is 0, 1 or 2;

q is 1 to 7;

r is 1 to 4;

t is 0 or 1 to 4;

wherein each alkyl, alkenyl, alkynyl, alkylene, linker, carbocyclic, heterocyclic, aryl and heteroaryl group may be optionally substituted with one or more optional substituents, and salts, esters and isomers thereof.

It should be understood that the cell which is treated according to a method of the present invention may be located ex vivo or in vivo. By “ex vivo” is meant that the cell has been removed from the body of a subject wherein the modulation of its activity will be initiated in vitro. For example, the cell may be a cell which is to be used as a model for studying any one or more aspects of the pathogenesis of conditions which are characterised by aberrant cell death signalling. In a particular embodiment, the subject cell is located in vivo.

In yet another aspect of the invention there is provided a method of treatment and/or prophylaxis of a pro-survival Bcl-2 member-mediated disease or condition in a mammal, comprising administering to said mammal an effective amount of a compound of formula (I):

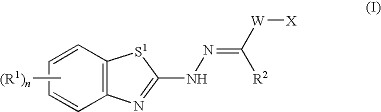

wherein

S¹ is S or Se;

W is an aryl or heteroaryl group;

X is -L-Z or -Q-Y—Z;

L is a linker of 1 to 3 atoms in length;

Q is an aryl or heteroaryl group;

Y is absent or is C₁₋₂alkylene;

Z is a carboxy group or an optionally substituted isosteric equivalent of a carboxy group;

R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyano and —N(R⁵)₂;

R² is selected from the group consisting of C₁₋₇alkyl, C₂₋₇alkenyl, C₂₋₇alkynyl, (CH₂)_{q}C₃₋₈cycloalkyl, (CH₂)_{q}halo, (CH₂)_{q}OH, C₁₋₇alkoxy(CH₂)ₜ, C₂₋₇alkenyloxy(CH₂)ₜ, C₂₋₇alkynyloxy(CH₂)ₜ, C₃₋₈cycloalkyloxy(CH₂)ₜ, C₁₋₇alkylthio(CH₂)ₜ, C₂₋₇alkenylthio(CH₂)ₜ, C₂₋₇alkynylthio(CH₂)ₜ, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CH, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl,

or R² together with a ring atom from W forms an optionally substituted 5-8 membered carbocyclic or heterocyclic ring;

each R⁴ is independently selected from the group consisting of hydrogen and halogen;

each R⁵ is independently selected from the group consisting of hydrogen and C₁₋₃alkyl;

each R⁶ is independently selected from the group consisting of hydrogen, C₁₋₇alkyl, C₂₋₇alkenyl, C₂₋₇alkynyl, C₃₋₈cycloalkyl, acyl, aryl, heterocyclyl or heteroaryl, or two R⁶ taken together with the nitrogen atom to which they are attached form an optionally substituted carbocyclic or heterocyclic ring;

R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃₋₈cycloalkyloxy, aryloxy, heteroaryloxy, heterocyclyloxy and N(R⁶)₂;

M is O, S or NR⁵;

n is 0, 1 or 2;

q is 1 to 7;

r is 1 to 4;

t is 0 or 1 to 4;

wherein each alkyl, alkenyl, alkynyl, alkylene, linker, carbocyclic, heterocyclic, aryl and heteroaryl group may be optionally substituted with one or more optional substituents, and salts, esters and isomers thereof.

In yet another aspect of the invention there is provided a method of treatment and/or prophylaxis of a disease or condition characterised by inappropriate persistence or proliferation of excess, unwanted or damaged cells in a mammal comprising administering to said mammal an effective amount of a compound of formula (I):

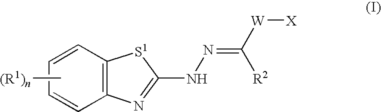

wherein

S¹ is S or Se;

W is an aryl or heteroaryl group;

X is -L-Z or -Q-Y—Z;

L is a linker of 1 to 3 atoms in length;

Q is an aryl or heteroaryl group;

Y is absent or is C₁₋₂alkylene;

Z is a carboxy group or an optionally substituted isosteric equivalent of a carboxy group;

R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyano and —N(R⁵)₂;

R² is selected from the group consisting of C₁₋₇alkyl, C₂₋₇alkenyl, C₂₋₇alkynyl, (CH₂)_{q}C₃₋₈cycloalkyl, (CH₂)_{q}halo, (CH₂)_{q}OH, C_{1 7}alkoxy(CH₂)ₜ, C₂₋₇alkenyloxy(CH₂)ₜ, C₂₋₇alkynyloxy(CH₂)ₜ, C₃₋₈cycloalkyloxy(CH₂)ₜ, C₁₋₇alkylthio(CH₂)ₜ, C₂₋₇alkenylthio(CH₂)ₜ, C₂₋₇alkynylthio(CH₂)ₜ, —(CH₂)_{q}aryl, —(CH₂)_{q}Oparyl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)₁NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)₂M(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl,

or R² together with a ring atom from W forms an optionally substituted 5-8 membered carbocyclic or heterocyclic ring;

each R⁴ is independently selected from the group consisting of hydrogen and halogen;

each R⁵ is independently selected from the group consisting of hydrogen and C₁₋₃alkyl;

each R⁶ is independently selected from the group consisting of hydrogen, C₁₋₇alkyl, C₂₋₇alkenyl, C₂₋₇alkynyl, C₃₋₈cycloalkyl, acyl, aryl, heterocyclyl or heteroaryl, or two R⁶ together with the nitrogen atom to which they are attached form an optionally substituted carbocyclic or heterocyclic ring;

R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃₋₈cycloalkyloxy, aryloxy, heteroaryloxy, heterocyclyloxy and N(R⁶)₂;

M is O, S or NR⁵;

n is 0, 1 or 2;

q is 1 to 7;

r is 1 to 4;

t is 0 or 1 to 4;

wherein each alkyl, alkenyl, alkynyl, alkylene, linker, carbocyclic, heterocyclic, aryl and heteroaryl group may be optionally substituted with one or more optional substituents, and salts, esters and isomers thereof

In still another aspect of the invention there is provided a use of a compound of formula (I):

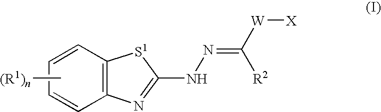

wherein

S¹ is S or Se;

W is an aryl or heteroaryl group;

X is -L-Z or -Q-Y—Z;

L is a linker of 1 to 3 atoms in length;

Q is an aryl or heteroaryl group;

Y is absent or is C₁₋₂alkylene;

Z is a carboxy group or an optionally substituted isosteric equivalent of a carboxy group;

R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyano and —N(R⁵)₂;

R² is selected from the group consisting of C₁₋₇alkyl, C₂₋₇alkenyl, C₂₋₇alkynyl, (CH₂)_{q}C₃₋₈cycloalkyl, (CH₂)_{q}halo, (CH₂)_{q}OH, C₁₋₇alkoxy(CH₂)ₜ, C₂₋₇alkenyloxy(CH₂)ₜ, C₂₋₇alkynyloxy(CH₂)ₜ, C₃₋₈cycloalkyloxy(CH₂)ₜ, C₁₋₇alkylthio(CH₂)ₜ, C₂₋₇alkenythio(CH₂)ₜ, C₂₋₇alkynylthio(CH₂)ₜ, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ₜM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl,

or R² together with a ring atom from W forms an optionally substituted 5-8 membered carbocyclic or heterocyclic ring;

each R⁴ is independently selected from the group consisting of hydrogen and halogen;

each R⁵ is independently selected from the group consisting of hydrogen and C₁₋₃alkyl;

each R⁶ is independently selected from the group consisting of hydrogen, C₁₋₇alkyl,

C₂₋₇alkenyl, C₂₋₇alkynyl, C₃₋₈cycloalkyl, acyl, aryl, heterocyclyl or heteroaryl, or two R⁶ taken together with the nitrogen atom to which they are attached form an optionally substituted carbocyclic or heterocyclic ring;

R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃₋₈cycloalkyloxy, aryloxy, heteroaryloxy, heterocyclyloxy and N(R⁶)₂;

M is O, S or NR⁵;

n is 0, 1 or 2;

q is 1 to 7;

r is 1 to 4;

t is 0 or 1 to 4;

wherein each alkyl, alkenyl, alkynyl, alkylene, linker, carbocyclic, heterocyclic, aryl and heteroaryl group may be optionally substituted with one or more optional substituents, and salts, esters and isomers thereof, in the manufacture of a medicament for regulating the death of a cell, or for inducing apoptosis in unwanted or damaged cells, or for the treatment and/or prophylaxis of a pro-survival Bcl-2 family member-mediated disease or condition, or for the treatment and/or prophylaxis of a disease or condition. characterised by inappropriate persistence or proliferation of unwanted or damaged cells.

The term “mammal” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). In a particular embodiment, the mammal is human or a laboratory test animal. In a particular embodiment, the mammal is a human.

As used herein, the term “disease or condition characterised by inappropriate persistence or proliferation of excess, unwanted or damaged cells” refers to diseases or conditions where there is inappropriate cell survival and excess, unwanted or damaged cells are not removed by the normal cellular process of apoptosis (programmed cell death). Such diseases include those in which there is aberrant, unwanted or inappropriate proliferation or prolonged survival of excess, unwanted or damaged cells, for example, where there is inactivation of apoptosis.

As used herein, the term “pro-survival Bcl-2 family member-mediated disease or condition” refers to diseases or conditions where excess, unwanted or damaged cells are not removed by normal cellular process, or diseases or conditions in which cells undergo aberrant, unwanted or inappropriate proliferation and/or survival. Such diseases include those related to inactivation of apoptosis (cell death), including disorders characterised by inappropriate cell proliferation or persistence.

Disorders characterised by inappropriate cell proliferation or persistence include, for example, inflammatory conditions such as acute and chronic inflammation arising from acute and chronic tissue injury including but not limited to, for example, acute lung injury, cancer including lymphomas, hyperplasias such as prostatic hyperplasia, autoimmune disorders, tissue hypertrophy etc.

Diseases or conditions associated with or characterised by inappropriate persistence or survival of unwanted or damaged cells include those relating to unwanted or damaged B cells, for example B cell non-Hodgkin's lymphoma, B cell acute lymphoblastic leukemia,

B cell chronic lymphocytic leukaemia, follicular leukaemia, as well as rheumatoid arthritis, systemic lupus erythematosis and related athropathies. Diseases and conditions associated with or characterised by the inappropriate persistence of unwanted or damaged T cells include T cell acute lymphoblastic leukemia, T cell non-Hodgkin's lymphoma and graft vs Host disease. Diseases and conditions associated with or characterised by the inappropriate persistence of unwanted or damaged myeloid cells include acute myelogenous leukemia, chronic myelogenous leukemia and chronic myelomonocytic leukemia. Diseases and conditions associated with or characterised by the inappropriate persistence of unwanted or damaged plasma cells include plasmacytomas, multiple myeloma, plasma cell dyscrasias and monoclonal gammapathy of unknown significance (MGUS). Diseases and conditions associated with or characterised by the inappropriate persistence of unwanted or damaged cancer cells, include benign and malignant cancers, especially cancer of the breast, prostate, ovary, testis, uterus, cervix, bladder, colon, small or large intestine, kidney, lung (including small cell lung cancer), oesophagus, gall bladder, pancreas, stomach, thyroid, skin, bone, bone marrow, lymph nodes, brain, throat, tongue, blood and liver.

Diseases or conditions associated with an excess of or prolonged survival of non-tumour cells include disorders involving excess platelets such as thrombocytosis and clotting disorders, for example stroke and heart attack, disorders involving excess red blood cells including polycythaemias, disorders involving excess white blood cells and disorders involving tissue hypertrophy.

An “effective amount” means an amount necessary at least partly to attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials. An effective amount in relation to a human patient, for example, may lie in the range of about 0.1 ng per kg of body weight to 1 g per kg of body weight per dosage. In a particular embodiment the dosage is in the range of 1μs to 1 g per kg of body weight per dosage, such as is in the range of 1 mg to 1 g per kg of body weight per dosage. In one embodiment, the dosage is in the range of 1 mg to 500mg per kg of body weight per dosage. In another embodiment, the dosage is in the range of 1 mg to 250 mg per kg of body weight per dosage. In yet another embodiment, the dosage is in the range of 1 mg to 100 mg per kg of body weight per dosage, such as up to 50 mg per kg of body weight per dosage. In yet another embodiment, the dosage is in the range of 1 μz to 1 mg per kg of body weight per dosage. Dosage regimes may be adjusted to provide the optimum therapeutic response. For example, several divided doses may be administered daily, weekly, monthly or other suitable time intervals, or the dose may be proportionally reduced as indicated by the exigencies of the situation.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the compounds of the invention or pharmaceutically acceptable salts or prodrugs thereof together with the subjection of the mammal to other agents or procedures which are useful in the treatment of diseases and conditions characterised by the inappropriate persistence or proliferation of unwanted or damaged cells. Compounds of the invention may be administered prior to, following or concomitantly with such other agents or procedures. For example, the compounds of the present invention may be administered in combination with other chemotherapeutic drugs, or with other treatments such as radiotherapy. Suitable chemotherapeutic drugs include, but are not limited to, cyclophosphamide, doxorubicine, etoposide phosphate, paclitaxel, topotecan, camptothecins, 5-fluorouracil, tamoxifen, staurosporine, avastin, erbitux, imatinib and vincristine.

While it is possible that, for use in therapy, a compound of the invention may be administered as a neat chemical, in a particular embodiment the active ingredient is presented as a pharmaceutical composition.

Thus, in a further aspect of the invention, there is provided a pharmaceutical composition comprising a compound of formula (I):

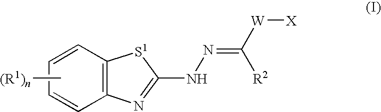

wherein

S¹ is S or Se;

W is an aryl or heteroaryl group;

X is -L-Z or -Q-Y—Z;

L is a linker of 1 to 3 atoms in length;

Q is an aryl or heteroaryl group;

Y is absent or is C₁₋₂alkylene;

Z is a carboxy group or an optionally substituted isosteric equivalent of a carboxy group;

R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyano and —N(R⁵)₂;

R² is selected from the group consisting of C₁₋₇alkyl, C₂₋₇alkenyl, C₂₋₇alkynyl, (CH₂)_{q}C₃₋₈cycloalkyl, (CH₂)_{q}halo, (CH₂)₁OH, C₁₋₇alkoxy(CH₂)ₜ, C₂₋₇alkenyloxy(CH₂)ₜ, C₂₋₇alkynyloxy(CH₂)ₜ, C₃₋₈cycloalkyloxy(CH₂)ₜ, C₁₋₇alkylthio(CH₂)ₜ, C₂₋₇alkenylthio(CH₂)ₜ, C₂₋₇alkynylthio(CH₂)ₜ, —(CH₂)_{q}aryl, —(CH₂)_{q}Oaryl, —(CH₂)_{q}Saryl, —(CH²)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl,

or R² together with a ring atom from W forms an optionally substituted 5-8 membered carbocyclic or heterocyclic ring;

each R⁴ is independently selected from the group consisting of hydrogen and halogen;

each R⁵ is independently selected from the group consisting of hydrogen and C₁₋₃alkyl;

each R⁶ is independently selected from the group consisting of hydrogen, C₁₋₇alkyl,

C₂₋₇alkenyl, C₂₋₇alkynyl, C₃₋₈cycloalkyl, acyl, aryl, heterocyclyl or heteroaryl, or two R⁶ taken together with the nitrogen atom to which they are attached form an optionally substituted carbocyclic or heterocyclic ring;

R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃₋₈cycloalkyloxy, aryloxy, heteroaryloxy, heterocyclyloxy and N(R⁶)₂;

M is O, S or NR⁵;

n is 0, 1 or 2;

q is 1 to 7;

r is 1 to 4;

t is 0 or 1 to 4;

wherein each alkyl, alkenyl, alkynyl, alkylene, linker, carbocyclic, heterocyclic, aryl and heteroaryl group may be optionally substituted with one or more optional substituents, and pharmaceutically acceptable salts, esters and isomers thereof,

and at least one pharmaceutically acceptable carrier.

The carrier(s) must be “acceptable” in the sense of being compatible with the other ingredients of the composition and not deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation. The compounds of the invention, together with a conventional adjuvant, carrier, excipient, or diluent, may thus be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed. Formulations containing ten (10) milligrams of active ingredient or, more broadly, 0.1 to two hundred (200) milligrams, per tablet, are accordingly suitable representative unit dosage forms. The compounds of the present invention can be administered in a wide variety of oral and parenteral dosage forms. It will be obvious to those skilled in the art that the following dosage forms may comprise, as the active component, either a compound of the invention or a pharmaceutically acceptable salt or derivative of the compound of the invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted in the shape and size desired.

In a particular embodiment the powders and tablets contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilizing agents, and the like.

For topical administration to the epidermis the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Formulations suitable for topical administration in the mouth include lozenges comprising active agent in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multidose form. In the latter case of a dropper or pipette, this may be achieved by the patient administering an appropriate, predetermined volume of the solution or suspension. In the case of a spray, this may be achieved for example by means of a metering atomising spray pump. To improve nasal delivery and retention the compounds according to the invention may be encapsulated with cyclodextrins, or formulated with their agents expected to enhance delivery and retention in the nasal mucosa.

Administration to the respiratory tract may also be achieved by means of an aerosol formulation in which the active ingredient is provided in a pressurised pack with a suitable propellant such as a chlorofluorocarbon (CFC) for example, dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP).

Conveniently the powder carrier will form a gel in the nasal cavity. The powder composition may be presented in unit dose form for example in capsules or cartridges of, e.g., gelatin, or blister packs from which the powder may be administered by means of an inhaler.

In formulations intended for administration to the respiratory tract, including intranasal formulations, the compound will generally have a small particle size for example of the order of 1 to 10 microns or less. Such a particle size may be obtained by means known in the art, for example by micronization.

When desired, formulations adapted to give sustained release of the active ingredient may be employed.

In a particular embodiment the pharmaceutical preparations are in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Liquids or powders for intranasal administration, tablets or capsules for oral administration and liquids for intravenous administration are particular compositions.

The invention will now be described with reference to the following Examples which illustrate some particular aspects of the present invention. However, it is to be understood that the particularity of the following description of the invention is not to supersede the generality of the preceding description of the invention.

### Examples

### Example 1

### Preparation of 5-[5-[1-(2-benzothiazolylhydrazono)ethyl]-2-furanyl]-2-chloro-benzoic acid (compound 4 from Table 1)

(a) Preparation of 5-(5-acetyl-2-furanyl)-2-chloro-benzoic acid

The arylation of 2-acetylfuran was carried out following the method of Oleinik et al.

2-Chloro-5-aminobenzoic acid (3.4 g, 20 mmol) was suspended in water (30 mL) and conc. hydrochloric acid (10 mL) was added with stirring. The mixture was cooled in ice and stirred during the dropwise addition of a solution of sodium nitrite (1.5 g) in water (8 mL). After about 15 minutes a solution of 2-acetylfuran (2.2 g, 20 mmol) in acetone (10 mL) and a solution of cupric chloride (1 g) in water (5 mL) were added dropwise and simultaneously to the ice cold diazonium salt solution. The mixture was allowed to warm to room temperature and left standing for 3 days during which time a dark oil separated. Most of the aqueous layer was decanted from the flask and the remaining dark oil was dissolved in dilute aqueous sodium hydroxide solution. The alkaline solution was washed with dichloromethane (50 mL) and then separated and acidified with dilute hydrochloric acid.

The acidic aqueous layer was extracted with dichloromethane (2×100 mL) and the organic layer was dried and evaporated to give the crude product (1.9 g) as a cream solid.

¹H nmr spectrum (CDCl₃) δ (ppm): 2.5 (s, 3H); 6.8 (d, 1H); 7.25 (d, 1H); 7.48 (d, 1H); 7.81 (d, 1H); 8.3 (s, 1H).

(b) 2-Hydrazinobenzothiazole was prepared by the reaction of 2-chlorobenzothiazole with hydrazine hydrate in ethanol [Katz, 1951].

(c) Preparation of Compound 4

5-(5-acetyl-2-furanyl)-2-chloro-benzoic acid (100 mg) was dissolved in hot ethanol (8 mL) and a solution of 2-hydrazinobenzothiazole (50 mg) in ethanol (4 mL) was added. The combined solution was stirred and heated under reflux for 1 hr and during this time a pale cream precipitate formed. The reaction was allowed to cool and then filtered to give the hydrazone product, compound 4, as a cream solid (85 mg). The ¹H nmr spectrum is summarized in Table 12 below.

### Example 2

Compounds No. 1, 2, 3, 5-15, 20-23, 26-35, 40 and 41 were each prepared in two stages by:

(a) reacting the appropriate alkanoylheterocycle, R²(O═C)-heterocycle, with the diazonium salt formed from the appropriate (substituted by group R⁷) 3-aminobenzoic acid using the Meerwein reaction conditions described in Example 1, part (a) above;

(b) condensation of the 3-(alkanoyl-heterocycle)-benzoic acid derivative formed above with 2-hydrazinobenzothiazole following similar reaction conditions to those described in Example 1, part (c) above.

The structures of the compounds were confirmed by the ¹H nmr spectra and in some cases also by MS and the ¹14 nmr data is summarized in Table 12 below.

### Example 3

### Preparation of 5-[5-[1-(2-benzothiazolylhydrazono)ethyl]-2-furanyl]-2-amino-benzoic acid (compound 16 from Table 1)

(a) A Meerwein arylation reaction was carried-out in the following manner: 5-amino-2-nitrobenzoic acid (2 g, 11 mmol) was dissolved in water (16 mL) and hydrochloric acid (5.6 mL). A solution of sodium nitrite (910 mg, 13.2 mmol) in water (5.2 mL) was added in portions at 0-5° C. and the reaction mixture was stirred at 5° C. for another 20 minutes. Solutions of 2-acetylfuran (1.21 g, 11 mmol) in acetone (7.2 mL) and copper(II)chloride dihydrate (598 mg) in water (3.8 mL) were added simultaneously to the diazonium salt solution at 5° C. with stirring. The reaction mixture was allowed to warm to room temperature and then stand overnight. The crude product which precipitated was purified by filtration and then trituration with ethyl acetate and petroleum ether. After drying in a vacuum oven, the 5-(5-acetyl-2-furanyl)-2-nitro-benzoic acid was obtained as a yellow solid (1.44 g, 48%).

(b) To a mixture of the above nitro compound (500 mg, 1.82 mmol) in methanol (40 mL) and ammonium formate (1.72 g, 27.3 mmol), palladium on carbon (10%, 220 mg) was added with stirring under nitrogen. After 2 hours at ambient temperature the contents were filtered through a plug of celite and washed with methanol. The filtrate was concentrated and the solid was suspended in water to wash off excess ammonium formate. The desired 2-aminobenzoic acid derivative was filtered and dried in a vacuum oven at 30° C. The product was obtained a brown solid (281 mg, 63%).

(c) To a solution of the above 2-amino-5-(5-acetyl-2-furyl)-benzoic acid (67 mg, 0.28 mmol) in ethanol (2 mL) benzothiazole-2-hydrazine (45 mg, 0.28 mmol) in ethanol (2-3 mL) was added and the solution was heated at 70-80° C. for 1 hour. After cooling, filtration and drying the desired hydrazone, Compound 16, was isolated (53 mg, 50%) and the nmr spectrum is listed in Table 12 below.

### Example 4

### Preparation of 5-[5-[1-(2-benzothiazolylhydrazono)ethyl]-2-furanyl]-2-benzamido-benzoic acid (compound 17 from Table 1)

(a) To a suspension of the 2-amino-5-(5-acetyl-2-furyl)-benzoic acid (70 mg, 0.29 mmol) described above in Example 3, part (b) above, in ethanol (2 mL) at 0° C., 1N sodium hydroxide (281 μL) was added, followed by the dropwise addition of benzoyl chloride (37 μL, 0.32 mmol). The mixture was slowly warmed to ambient temperature over 3 hours. After concentrating the reaction mixture, it was separated between 1N HCl and ethyl acetate. The organic layer was washed with brine, dried and after filtration the solvent was removed under vaccum. The crude product was suspended in minimum of methanol and the desired product was filtered off as a solid. On drying, the benzamide derivative was obtained (90 mg, 90%).

(b) The hydrazone of this acylated intermediate was obtained following a similar method to that described in Example 1, part (c). Thus 31 mg (0.09 mmol) of the benzamide product from part (a) above was condensed with 15 mg (0.09 mmol) of benzothiazole-2-hydrazine in ethanol (2 mL). The desired hydrazone, Compound 17, was isolated as a white solid (32 mg, 71%) and the ¹H nmr is summarized in Table 12.

### Example 5

Compound Nos. 18, 19, 24, 25 and 36-38 were each prepared in two stages by acylation of 2-amino-5-(5-acetyl-2-furyl)-benzoic acid with the appropriate acid chloride following a similar method to that described in Example 4 above. The compounds were characterized by their ‘H nmr and/or mass spectra, a summary of which is provided in Table 12.

### Example 6

### Preparation of [4-[1-(2-benzothiazolylhydrazono)ethyl]-phenoxy]acetic acid (compound 200 from Table 2)

Benzothiazole-2-hydrazine (33 mg, 0.2 mmol) was dissolved in ethanol (3 mL) with warming and added to a solution of 4-acetylphenoxyacetic acid (40 mg, 0.2 mmol) in ethanol (4 mL). The solution was heated at reflux for 1 hour and then allowed to cool and stand at room temperature. The precipitate which separated was collected by filtration and air dried to give compound 200 (60 mg, 85%). The ¹H nmr spectrum is summarized in Table 12 below.

### Example 7

Compounds No. 201-207, 210, 211 and 212 were each prepared by reaction of benzothiazole-2-hydrazine with the appropriate ketone following a similar method to that described in Example 6 above. The compounds were characterized primarily by their ¹H nmr spectra, a summary of which is provided in Table 12.

### Example 8

### Preparation of N-Methanesulphonyl-5-[5-[1-(2-benzothiazolylhydrazono)ethyl]-2-(N-methylpyrrolyl]-2-chloro-benzamide (compound 301 from Table 3)

(a) Similar conditions to the Meerwein arylation described in Example 1 were used as follows: 5-amino-2-chlorobenzoic acid (697 mg, 4.1 mmol) was dissolved in water (6.55 mL) and hydrochloric acid (2.06 mL) and then cooled to 0-5° C. with ice and diazotized by slow addition of a solution of sodium nitrite (336 mg, 4.9 mmol) in water (1.13 mL). To the diazonium species generated in-situ, a solution of 2-acetyl-1-methylpyrrole (500 mg, 4.1 mmol) in acetone (2.67 mL) was added followed by copper(II)chloride dihydrate (221 mg, 1.3 mmol) in water (1.40 mL). The reaction mixture was allowed to warm to room temperature and then stand for 24 hours. The precipitate which formed was collected by filtration and then triturated with petroleum ether and ethyl acetate to give the pure 2-chloro-5-(2-acetyl-N-methylpyrrolyl)-benzoic acid product (787 mg, 70%).

(b) The pyrrolyl-benzoic acid from part (a) above (50 mg, 0.18 mmol) was dissolved in anhydrous dichloromethane (3 mL) and to this solution was added methanesulfonamide (14 mg, 0.15 mmol), EDCI (43 mg, 0.23 mmol) and DMAP (27 mg, 0.23 mmol) at ambient temperature and with stirring. After 16 hours the reaction mixture was concentrated and partitioned between 1N HCl and ethyl acetate. The organic layer was separated and washed with brine, dried (MgSO₄) and the solvent removed after filtration. The crude product was purified by column chromatography on silica (eluting with 5/1 dichloromethane:methanol) to give the 2-chloro-5-(2-acetyl-N-methylpyrrolyl)-N-methanesulfonylbenzamide product (21 mg, 33%).

(c) Reaction of the above ketone (14 mg, 0.038 mmol) with benzothiazole-2-hydrazine (6 mg, 0.038 mmol) in hot ethanol (1-2 mL) gave the benzothiazole-hydrazone (11 mg, 48%, Compound 301) as a cream solid. The hydrazone was characterized by ¹ H nmr and the spectrum is summarized in Table 12 below.

### Example 9

The acylsulfonamide Compounds No. 208-209, 303, 310, 311, 314-316, 337, 338 and 347-350 were prepared from the appropriate benzoic acid following a similar method to that described in Example 8, parts (b) and (c). The compounds were characterized primarily by their ¹H nmr spectra, a summary of which is provided in Table 12.

### Example 10

### Preparation of 3-[5-[-1-(2-benzothiazolylhydrazono)ethyl]-2-furanyl]-phenylacetic acid (compound 302 from Table 3)

Compound No. 302 was prepared in two stages by: (a) reacting acetylfuran with the diazonium salt formed from 3-aminophenylacetic acid using the Meerwein reaction conditions described in Example 1, part (a) above;

(b) condensation of the 3-(5-acetyl-2-furyl)-phenylacetic acid formed above with 2-hydrazinobenzothiazole following similar reaction conditions to those described in Example 1, part (c) above. The structure was confirmed by the ¹H nmr spectra and the ¹H nmr data is summarized in Table 12 below.

### Example 11

### Preparation of 3-{5-[1-(Benzothiazol-2-yl-hydrazono)-ethyl]-furan-2-yl}-N-(3-phenyl-propanoyl)-benzenesulfonamide (Compound 304 from Table 3)

(a) 3-aminobenzenesulfonamide

3-nitrobenzenesulfonamide (1.01 g, 5 mmol) was suspended in 10 mL of MeOH. THF was then added until solids dissolved. Ammonium acetate (1.45 g, 23 mmol) and Pd/C (10%, 205 mg) were then added successively at 0° C. The temperature was kept at 0-5° C. until all gaseous evolution had ceased. The reaction was then warmed up to room temperature and stirred for 2 hours. The reaction mixture was then filtered through a pad of celite and the solid residue was rinsed thoroughly with THF. The filtrate was concentrated affording 3-aminobenzenesulfonamide as a white solid that was used in the next without further purification (810 mg, 93%). MS (+ESI): 173.0 (M+H⁺).

(b) 3-(5-Acetyl-furan-2-yl)-benzenesulfonamide

3-aminobenzenesulfonamide (750 mg, 4.36 mmol) was suspended in 6.8 mL of water. Concentrated hydrochloric acid (2.16 mL) was then added followed by dropwise addition of an aqueous solution of sodium nitrite (391 mg, 5.67 mmol in 2.1 mL) at 0° C. The reaction was stirred at 0° C. for 15 minutes. Acetyl furan (437 μL, 4.36 mmol) in 3 mL of acetone was added followed by copper chloride hydrate (238 mg, 1.4 mmol) in 2 mL of water. The reaction was then slowly allowed to warm to room temperature and stirred for 72 hours by which time a brown solid had formed. The volatile solvents were evaporated and a mixture of dichloromethane and ethyl acetate was added to the remaining aqueous solution. The mixture was triturated. Solvents were removed and more ethyl acetate was added. The solid was then isolated by filtration. When the purity of the compound is low, it can be recrystallised at this stage using a mixture petroleum ether/ethyl acetate. ¹H NMR (d₆-DMSO, ppm): 8.22 (s, 1H), 8.05 (d, J=7.88 Hz, 1H), 7.81 (d, J=7.86 Hz, 1H), 7.67 (t, J=7.82 Hz, 1H), 7.57 (d, J=3.72 Hz, 1H), 7.47 (s, 2H), 7.29 (d, J=3.74 Hz, 1H), 2.45 (s, 3H). MS (+ESI): 266.1 (M+H⁺).

(c) 3-(5-Acetyl-furan-2-yl)-N-(3-phenyl-propionyl)-benzenesulfonamide

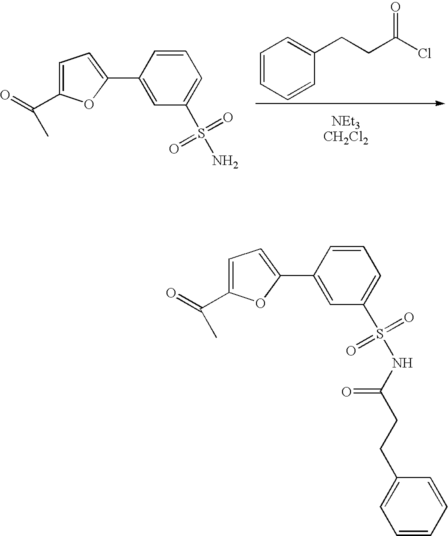

Hydrocinnamoyl chloride (135 μL, 1 mmol) was added to a mixture of 3-(5-Acetyl-furan-2-yl)-benzenesulfonamide and triethylamine (153 μL, 1 mmol) in 3 mL of dichloromethane. The reaction mixture was stirred at room temperature for 16 hours. The mixture is then diluted with dichloromethane and rinsed with 1N HCl (three times), water, brine and dried over Na₂SO₄. Concentration afforded a yellow thick oil. The residue was purified by flash chromatography on silica using a 98:2 mixture of dichloromethane/methanol. The product was obtained as a thick yellow oil (145 mg, 73%). NMR (CDCl₃, ppm): 8.79 (br s, 1H), 8.35 (s, 1H), 8.02-7.98 (m, 2H), 7.55 (t, J=7.94 Hz, 1H), 7.28 (d, J=3.68 Hz, 1H), 7.21-7.08 (m, 3H), 7.05-7.03 (m, 2H), 6.90 (d, J=3.71 Hz, 1H), 2.88 (t, J=7.47, 2H), 2.60 (t, J=7.73 Hz, 2H), 2.54 (s, 3).

(d) 3-{5-[1-(Benzothiazol-2-yl-hydrazono)-ethyl]-furan-2-yl}-N-(3-phenyl-propanoyl)-benzenesulfonamide

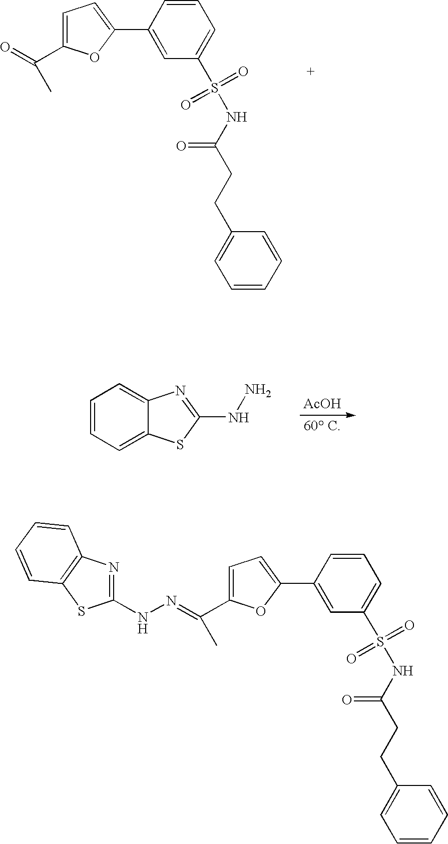

A mixture of 3-(5-Acetyl-furan-2-yl)-N-(3-phenyl-propanoyl)-benzenesulfonamide (43 mg, 0.11 mmol) and benzothiazole-2-hydrazine (20 mg, 0.12 mmol) in 1 mL of acetic acid was heated at 60° C. for 16 hours. After this time a yellow solid had precipitated. It was collected by filtration, rinsed with a small amount of acetic acid and diethyl ether and dried in a vacuum oven to give the title compound (30 mg, 55%). The NMR spectrum is summarised in Table 12 (compound 304).

### Example 12

### 3-(5-[1-(Benzothiazol-2-yl-hydrazono)-ethyl]-furan-2-yl}-N-(4-phenyl-butyryl)-benzenesulfonamide (Compound No 305 in Table 3)

(a) 3-(5-Acetyl-furan-2-yl)-N-(4-phenyl-butyryl)-benzenesulfonamide

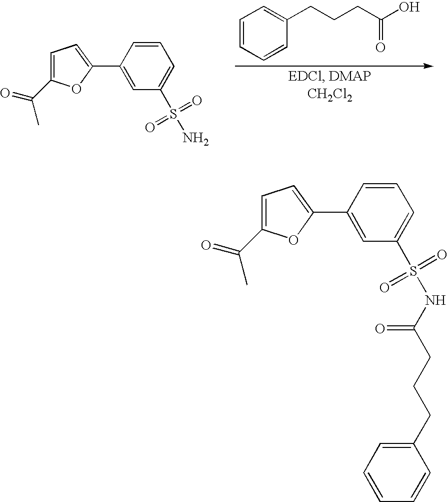

EDCI (190 mg, 1 mmol) and DMAP (127 mg, 1 mmol) were added to a mixture of 3-(5-acetyl-furan-2-yl)-benzenesulfonamide (133 mg, 0.5 mmol) and 4-phenylbutyric acid (82 mg, 0.5 mmol) in 4 mL of dichloromethane. The reaction was stirred at room temperature for 16 hours. The same work up and purification method as described in Example 11, part (c) was applied to obtain the product as a thick yellow oil (154 mg, 75%). ¹H NMR (CDCl₃, ppm): 8.49 (br s, 1H), 8.38 (s, 1H), 8.02 (t, J=7.04 Hz, 2H), 7.59 (t, J=7.87 Hz, 1H), 7.27 (d, J=3.71 Hz, 1H), 7.23-7.13 (m, 3H), 7.07-7.04 (m, 2H), 6.90 (d, J=3.71 Hz, 1H), 2.56 (t, J=7.36, 2H), 2.53 (s, 3H), 2.56 (t, J=7.32 Hz, 2H), 1.90 (q, J=7.67 Hz, 2H).

(b) 3-{5-[1-(Benzothiazol-2-yl-hydrazono)-ethyl]-furan-2-yl}-N-(4-phenyl-butyryl)-benzenesulfonamide (Compound No 305 in Table 3)

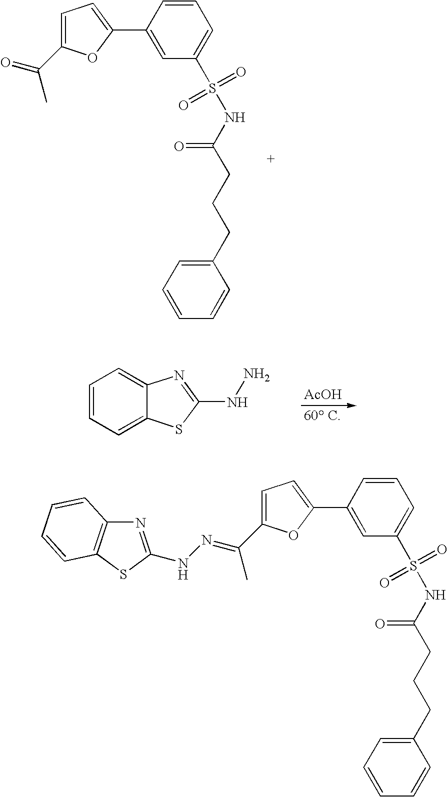

Following essentially the same method as Example 11, part (d) with the starting materials 3-(5-acetyl-furan-2-yl)-N-(3-phenyl-butyryl)-benzenesulfonamide (80 mg, 0.19 mmol) and benzothiazole-2-hydrazine (36 mg, 0.22 mmol) gave the title compound as a yellow solid (63 mg, 59%). The NMR spectrum is summarised in Table 12.

### Example 13

The acylsulfonamide Compounds No. 300, 307-309, 312, 313, 317-319, 321-328, 332-336, 341-343, 351-354, 356-359, 362-369, 375-379, 382 and 503 were each prepared by coupling of the appropriate sulfonamide and carboxylic acid following one of the methods described in Examples 11 and 12. The compounds were characterized primarily by their ¹H nmr spectra, a summary of which is provided in Table 12.

### Example 14

### N-Acetyl-3-{5-[1-(benzothiazol-2-yl-hydrazono)-ethyl]-thiophen-2-yl}-benzenesulfonamide (Compound No. 306 in Table 3)

(a) 3-(5-Acetyl-thiophen-2-yl)-benzenesulfonamide

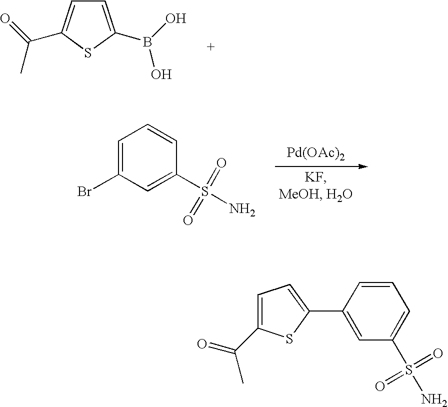

A mixture of 3-bromobenzenesulfonamide (118 mg, 0.5 mmol), 5-acetyl-thiophen-2-boronic acid (170 mg, 1 mmol), potassium fluoride (174 mg, 3 mmol) in 5 mL of a 9:1 MeOH/water mixture was degassed by passing nitrogen gas through the solution. Palladium acetate (5 mg, 4 mol %) was then added and the reaction immediately brought to 70° C. by immersing the flask in an oil bath. The reaction mixture was stirred at this temperature under nitrogen for 3 hours. The reaction was concentrated and the residue was purified by flash chromatography on SiO₂ using dichloromethane/ethyl acetate 95:5 to 90:10. A yellow solid was obtained (91 mg, 65%).

(b) N-Acetyl-3-(5-acetyl-thiophen-2-yl)-benzenesulfonamide

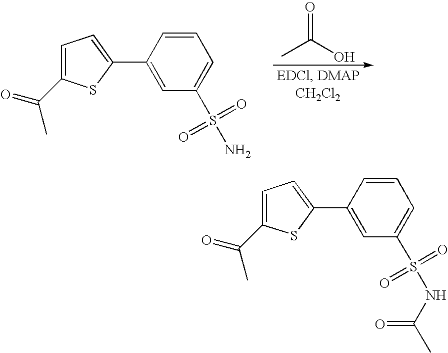

The method described in Example 12, part (a) was followed with 3-(5-acetyl-thiophen-2-yl)-benzenesulfonamide (23 mg, 0.08 mmol) and acetic acid (7 μL, 0.084 mmol). Purification on SiO2 using CH₂Cl₂/MeOH 98:2 afforded the title acylsulfonamide as a yellow solid (18 mg, 68%). ¹H NMR (d₆-DMSO, ppm): 12.2 (br s, 1H), 8.16 (t, J=1.65 Hz, 1H), 8.10 (d, J=8.01 Hz, 1H), 7.98 (d, J=4.02 Hz, 1H), 7.91 (d, J=7.89 Hz, 1H), 7.73 (d, J=4.14 Hz, 1H), 7.71 (d, J=7.89 Hz, 1H), 2.56 (s, 3H), 1.94 (s, 3H). MS (−ESI): M−H⁻: 322.3.

(c) N-Acetyl-3-{5-[1-(benzothiazol-2-yl-hydrazono)-ethyl]-thiophen-2-yl}-benzenesulfonamide

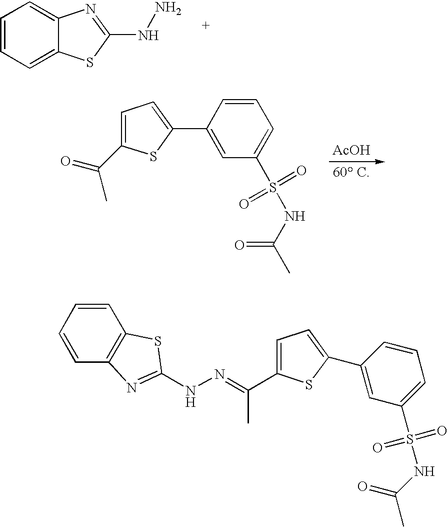

Condensation of benzothiazole-2-hydrazine with N-acetyl-3-(5-acetyl-thiophen-2-yl)-benzenesulfonamide (23 mg, 0.08 mmol) was carried out following the method described in Example 11, part (d). Compound 306 was obtained as a yellow solid (18 mg, 68%). The ¹H NMR spectrum (d₆-DMSO) summarized in Table 12 indicated that the compound was obtained as a 2:1 mixture of trans/cis hydrazone isomers.

### Example 15

### 3-(5-[1-(Benzothiazol-2-yl-hydrazono)-ethyl]furan-2-yl}-N-(4-phenyl-2-aminobutyryl)-benzenesulfonamide trifluoroacetate (Compound No. 320)

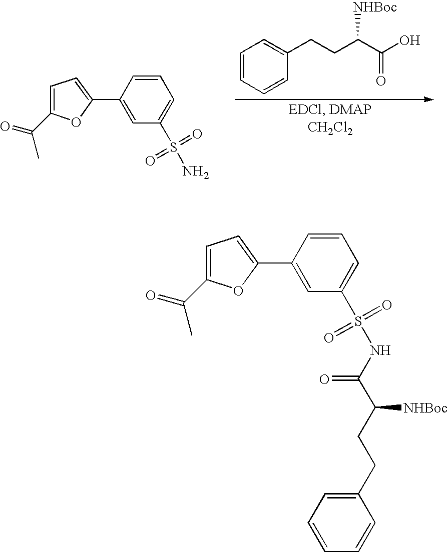

(a) 3-{5-[1-(Benzothiazol-2-yl-hydrazono)-ethyl]-furan-2-yl}-N-(4-phenyl-2-tert-butoxycarbonylaminobutyryl)-benzenesulfonamide

The acylsulfonamide intermediate was prepared by reaction of L-Boc-homophenylalanine (143 mg, 0.51 mmol) and 3-(5-acetyl-furan-2-yl)-benzenesulfonamide (135 mg, 0.51 mmol) following the coupling conditions described in Example 12, part (a). The compound was obtained as a yellow solid (170 mg, 63%).

(b) 3-{5-[1-(Benzothiazol-2-yl-hydrazono)-ethyl]-furan-2-yl}-N-(4-phenyl-2-aminobutyryl)-benzenesulfonamide trifluoroacetic acid salt

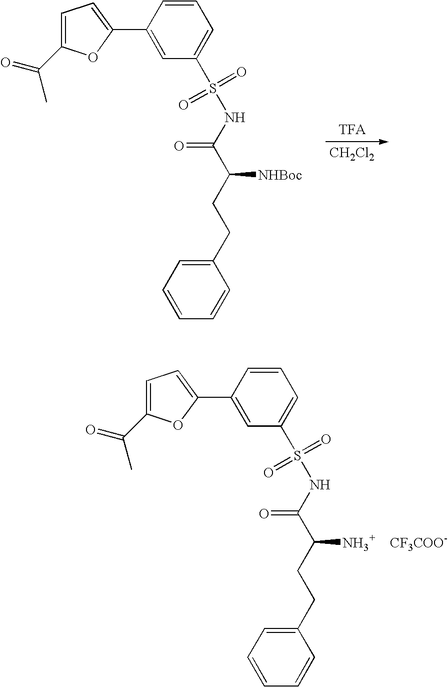

3-{5-[1-(Benzothiazol-2-yl-hydrazono)-ethyl]-furan-2-yl}-N-(4-phenyl-2-tert-butoxycarbonylaminobutyryl)-benzenesulfonamide (170 mg, 0.32 mmol) was treated with 5 mL of a 20% solution of trifluoroacetic acid in dichloromethane. The reaction was stirred at room temperature for 16 hours. After this time the reaction was concentrated. The residue was taken up in toluene and the mixture concentrated. This procedure was repeated three times. The yellow solid obtained was dried under vacuum (179 mg, quantitative).

(c) 3-{5-[1-(Benzothiazol-2-yl-hydrazono)-ethyl]-furan-2-yl}-N-(4-phenyl-2-aminobutyryl)-benzenesulfonamide trifluoroacetate

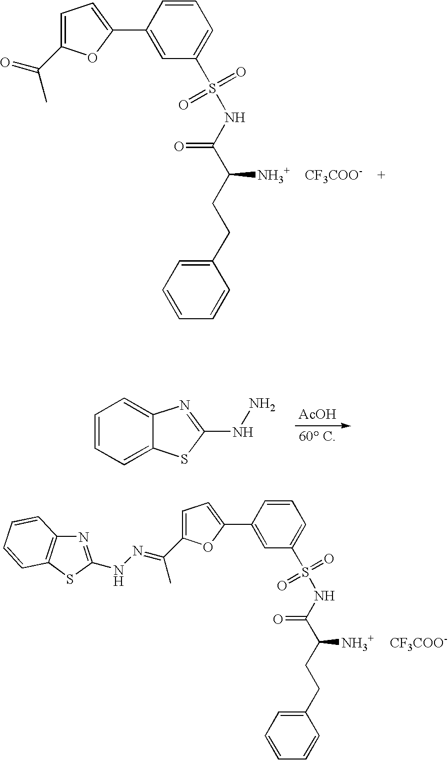

Following the typical procedure described in Example 11, part (d) reaction of 3-{5-[1-(Benzothiazol-2-yl-hydrazono)-ethyl]-furan-2-yl}-N-(4-phenyl-2-aminobutyryl)-benzenesulfonamide trifluoroacetate (173 mg, 0.32 mmol) and benzothiazole-2-hydrazine (87.4 mg, 0.33 mmol) gave the title compound as a yellow solid (93 mg, 42%). ¹H NMR (d₆-DMSO, ppm): indicates a 2:1 mixture of isomers. 8.19 (s, 1H), 7.97 (br s, 1H), 7.90 (d, J=7.89 Hz, 1H), 7.80 (d, J=7.87 Hz, 1H), 7.65 (d, J=6.18 Hz, 1H), 7.57 (t, J=7.80 Hz, 1H), 7.26-7.17 (m, 3H), 7.14-7.12 (m, 2H), 7.04-7.02 (m, 3H), 7.98 (d, J=3.56, 1H), 3.60 (br s, 3H), 2.56-2.46 (m, 1H), 2.38-2.28 (m, 2H), 1.94-1.87 (m, 2H). MS (+ESI): M⁺⁻: 574.2.

### Example 16

The acylsulfonamide Compounds No. 329-331, 339, 340, 344-346, 360, 361, 370-374, 380, 381 and 383-386 were prepared by starting with the appropriate Boc-protected amino acid and following a similar set of reactions to those described in Example 15. The compounds were characterized primarily by their ‘H nmr spectra, a summary of which is provided in Table 12.

### Example 17

### 3′[1-(Benzothiazol-2-yl-hydrazono)-ethyl]-biphenyl-3-carboxylic acid (Compound No. 500 in Table 5)

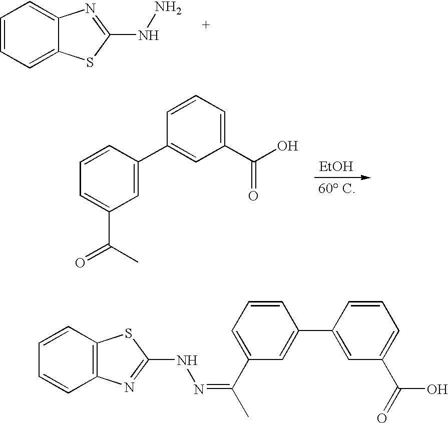

3′-Acetyl-biphenyl-3-carboxylic acid (120 mg, 0.5 mmol) and 2-hydrazinobenzothiazole (91 mg, 0.55 mmol) were suspended in ethanol (1.5 mL) and heated at 60° C. for 2.5 hours. The reaction was then cooled down and the precipitate was filtered and rinsed with ethanol. The title compound was obtained as a white solid (140 mg,.72%). The ¹H NMR spectrum is summarised in Table 12.

### Example 18

(a) (3-Bromo-phenyl)-methanesulfonamide

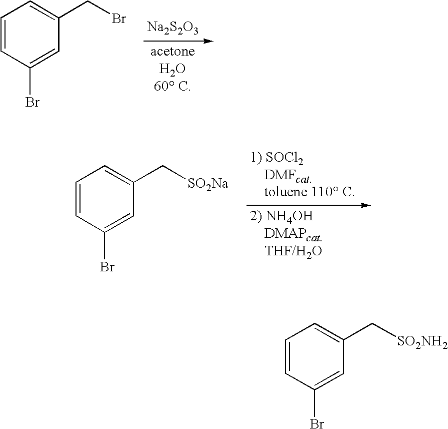

3-bromobenzylbromide (3 g, 12 mmol) dissolved in 6 mL of acetone was added to a solution of sodium sulfite (2.89 g, 18.3 mmol) in 9 mL of water. The reaction was heated at 60° C. for 16 hours. The mixture was concentrated and the white solid obtained was dried in a vacuum oven. This solid was then suspended in 42 mL of toluene with a few drops of DMF. Thionyl chloride (9 mL, 123 mmol) was then added and the reaction was heated at 110° C. for 15 hours. The deep yellow mixture was cooled to room temperature and poured onto crushed ice and ethyl acetate. The organic phase was washed two times with cold water, then brine and dried over MgSO₄. Concentration afforded a pasty solid which was dissolved in 10 mL of THF. This solution was added to a mixture of 25% ammonium hydroxide aqueous solution in 10 mL of THF with few crystals of DMAP. The reaction was stirred for 1 hour at room temperature. The reaction was then poured onto a water/ethyl acetate mixture. The aqueous phase was extracted three times with ethyl acetate. The combined organic phases were washed with saturated NaHCO₃ and brine, dried over Na₂SO₄ and concentrated. An off-white solid was obtained. It was triturated with Et₂O several times and recrystallised from water to afford (3-Bromo-phenyl)-methanesulfonamide as colorless needles (3.1 g, 57% over three steps). ¹H NMR (d₆-DMSO, ppm): 7.58 (t, J=1.81 Hz, 1H), 7.51 (dt, J=8.11, 1.17 Hz, 1H), 7.37 (d, J=7.69 Hz, 1H), 7.26 (t, J=7.81 Hz, 1H), 4.58 (br s, 2H), 4.28 (s, 2H).

(b) [3-(5-Acetyl-thiophen-2-yl)-phenyl]-methanesulfonamide

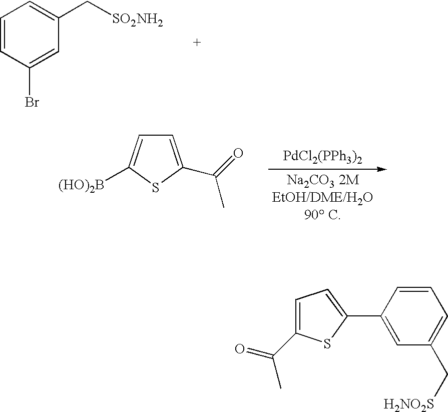

A mixture of (3-Bromo-phenyl)-methanesulfonamide (0.5 g. 2 mmol), 2-acetyl-thiophen-5-boronic acid (180 mg, 4 mmol), PdCl₂ (PPh₃)₂ (88 mg, 5 mol %), sodium carbonate (2.63 mL of 2M aqueous solution) in a 1:1:1 DME/EtOH/H2O mixture was heated at 90° C. for 4 hours. The reaction was cooled down, diluted with ethyl acetate and poured onto water. The aqueous phase was extracted three times with ethyl acetate. The combined organic phases were washed with water and brine, dried over MgSO₄ and concentrated. The residue was recrystallised in ethanol. The product was obtained as brown crystals (274 mg, 39%). ¹H NMR (d₆-DMSO, ppm): 7.92 (d, J=4 Hz, 1H), 7.73-7.71 (m, 2H), 7.60 (d, J=4.57 Hz, 1H), 7.46 (t, J=7.66 Hz, 1H), 7.37 (d, J=7.69 Hz, 1H), 6.86 (s, 2H), 4.31 (s, 2H), 2.51 (s, 3H).

(c) N-Acetyl-C-[3-(5-acetyl-thiophen-2-yl)-phenyl]-methanesulfonamide

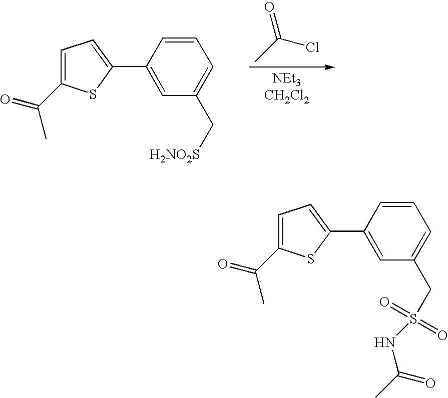

The product was obtained following the procedure described in Example 11c using [345-Acetyl-thiophen-2-yl)-phenyl]-methanesulfonamide and acetyl chloride. MS (−ESI): M−H: 336.3.

(d) N-(4-phenyl)butyryl-C-[3-(5-acetyl-thiophen-2-yl)-phenyl]-methanesulfonamide

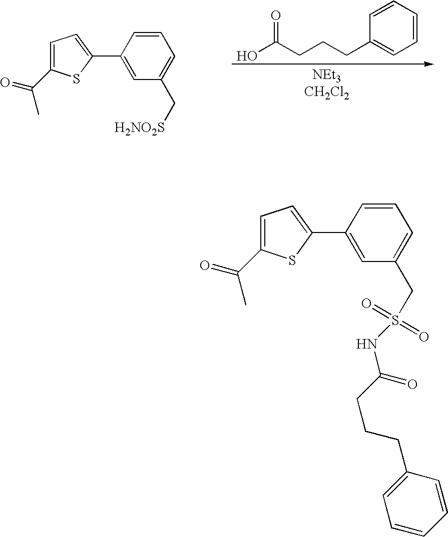

The product was obtained following the procedure described in Example 12a using [3-(5-Acetyl-thiophen-2-yl)-phenyl]-methanesulfonamide and 4-phenylbutyric acid. NMR (CDCl₃, ppm): 7.92 (d, J=4 Hz, 1H), 7.73-7.71 (m, 2H), 7.60 (d, J=4.57 Hz, 1H), 7.46 (t, J=7.66 Hz, 1H), 7.37 (d, J=7.69 Hz, 1H), 6.86 (s, 2H), 4.31 (s, 2H), 2.51 (s, 3H). MS (−ESI): M−H: 336.3.

### Example 19

### Preparation of 5-[3-[1-(2-benzothiazolylhydrazono)ethyl]-phenyl]-2-furoic acid (compound 400 from Table 4)

(a) Preparation of 5-(3-acetylphenyl)-2-furoic acid

The arylation of furoic acid was carried out by diazotisation of 3-amino-acetophenone following a similar method to that described in Example 1, part (a). Thus a solution of 3-amino-acetophenone (1.4 g, 10 mmol) in water (15 mL) and concentrated hydrochloric acid (6 mL) was cooled to 0-5° C. and a solution of sodium nitrite (0.84 g, 12 mmol) in water (4 mL) was added slowly, keeping the reaction temperature below 5° C. After completion of the addition the clear pale brown diazonium salt solution was stirred at 0-5° C. for 15 minutes and then solutions of furoic acid (1.1 g, 10 mmol) in acetone (6 mL) and cupric chloride dihydrate (0.5 g) in water (3 mL) were added slowly and simultaneously. The reaction mixture was then allowed to warm slowly to room temperature and stand overnight. The brown oily mixture was extracted with dichloromethane (2×50 mL) and the organic layer was separated, dried (MgSO₄) and concentrated to give a dark oil (1.5 g). The crude product was partially purified by chromatography on silica using chloroform and then 1% methanol/chloroform as eluent to give 5-(3-acetylphenyl)-2-furoic acid as a still impure brown solid (70 mg, 3%). The ¹H nmr spectrum (CDCl₃) 8 (ppm): 2.65 (s, 3H); 6.87 (d, 1H); 7.2-8.0 (m, 18H); 8.33 (s, 1H) showed the presence of the title compound.

(b) Preparation of Compound 400

The impure 5-(3-acetylphenyl)-2-furoic acid (70 mg) was dissolved in hot ethanol (5 mL) and a solution of 2-hydrazinobenzothiazole (40 mg) in ethanol (3 mL) was added. The combined solution was stirred and heated under reflux for 1 hr and during this time a pale cream precipitate formed. The reaction was allowed to cool and then filtered to give the hydrazone product, compound 400, as a cream solid (35 mg), LC/MS showed the major component to have mass m/e 378 (M+1). The ¹H nmr spectrum is summarized in Table 12 below.

### Example 20

### Preparation of 5-[3-[1-(2-benzothiazolylhydrazono)ethyl]phenyl]-2-thiophenecarboxylic acid (compound 401 from Table 4)

(a) Preparation of 5-(3-acetylphenyl)-2-thiophenecarboxylic acid

The first step towards this target was a coupling reaction between 5-bromo-2-thiophenecarboxylic acid (100 mg, 0.481 mmol) and 3-acetylphenylboronic acid (84 mg, 0.511 mmol). Both of the above were taken-up in a mixture of methyl alcohol and water (1:1, 6 mL). Palladium on carbon (10%, 12 mg) and sodium carbonate (98 mg, 0.924 mmol) were added to the mixture and heated under reflux for two hours. Once the reaction was complete, the contents were filtered through a plug of celite and washed through with methyl alcohol. After removal of solvent, the residue was suspended in 10 mL water and acidified with 2N HCl whilst cooling in an ice-bath. A white solid precipitated which was filtered, and dried in a vacuum oven at 30° C. Isolated 97 mg (82%) of pure product.

(b) Preparation of Compound 401

5-(3-acetylphenyl)-2-thiophenecarboxylic acid (50 mg) was suspended in hot ethanol (2 mL) with 2-hydrazinobenzothiazole (33 mg). The mixture was stirred and heated under reflux for 1 hr and during this time a pale cream precipitate formed. The reaction was allowed to cool and then filtered to give the hydrazone product, compound 401, as a cream solid (69 mg, 86%) The ¹H nmr spectrum is summarized in Table 12 below.

### Example 21

### Preparation of 3-[4-[1-(2-benzothiazolylhydrazono)ethyl]-5-hydroxy-3-methyl-pyrazol-1-yl]-benzoic acid (compound 501 from Table 5)

(a) Preparation of 3-(5-hydroxy-3-methylpyrazol-1-yl)-benzoic acid

Ethyl acetoacetate (1.3 mL, 10 mmol) and 3-hydrazino-benzoic acid (1.5 g, 10 mmol) were dissolved in ethanol (20 mL) and the solution was stirred and heated under reflux for 5 hours, by which time thin layer chromatography (tlc) indicated that the reaction was complete. The mixture was poured into cold water to give an oily precipitate which was collected and re-dissolved in dilute aqueous sodium bicarbonate solution. The solution was cooled and acidified slowly with dilute hydrochloric acid to give a pale orange solid which was collected by filtration to give the crude pyrazole product (2.1 g, 95%) ¹H nmr spectrum (d₆DMSO) δ (ppm): 2.09 (s, 3H); 5.36 (s, 1H); 7.50 (dd, 1H); 7.72 (d, 1H); 7.95 (d, 1H); 8.29 (s, 1H).

(b) Preparation of 3-(4-acetyl-5-hydroxy-3-methylpyrazol-1-yl)-benzoic acid

3-(5-hydroxy-3-methylpyrazol-1-yl)-benzoic acid (0.4 g, 2mmol), acetic anhydride (400 μL, 4 mmol) and 4-dimethylaminopyridine (40 mg) were added to toluene (20 mL) with stirring. The mixture was stirred and heated at reflux for 4 hours at which time tic indicated that all the starting material was gone and a new slower running material had formed. The mixture was shaken with dilute hydrochloric acid and the organic layer was separated, dried (MgSO₄) and concentrated to give the crude product as an orange foam. ¹H nmr spectrum (d₆DMSO) δ (ppm): 2.38 (s, 3H); 2.40 (s, 3H); 7.56 (dd, 1H); 7.78 (d, 1H); 7.99 (d, 1H); 8.29 (s, 1H). Mass spectrum (ESI⁺): m/e 261 (M+1).

(c) Preparation of Compound 501

3-(4-acetyl-5-hydroxy-3-methylpyrazol-1-yl)-benzoic acid (65 mg) was dissolved in hot ethanol (8 mL) and 2-hydrazinobenzothiazole (42 mg) in ethanol (3 mL) was added. The clear solution was stirred and heated under reflux for 1 hr and during this time a pale cream precipitate formed. The reaction was allowed to cool and stand overnight and then filtered to give the hydrazone product, compound 501, as a cream solid (78 mg, 80%), mass spectrum(ESI⁺) gave m/e 408 (M+1). The ¹H nmr spectrum is summarized in Table 12 below.

### Example 22

### Preparation of 3-{5-[1-(2-benzothiazolylhydrazono)ethyl]-2-thiophenyl}-2-methyl-benzoic acid (Compound No. 39 from Table 1)

(a) 3-(5-Acetyl-thiophen-2-yl)-2-methyl-benzoic acid

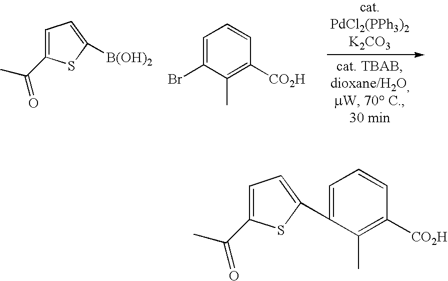

A mixture of 3-Bromo-2-methyl benzoic acid (300 mg, 1.4 mmol), 2-acetyl-thiophen-5-boronic acid (332 mg, 1.96 mmol), PdCl₂(PPh₃)₂ (49 mg, 5 mol %), tetrabutylammonium bromide (89 mg, 0.14 mmol), potassium carbonate (578 mg, 4.2 mmol) in a dioxane:H₂O mixture (5:2; 5 mL) was heated at 70° C. under microwave irradiation for 1 hour. The reaction was cooled, diluted with ethyl acetate and poured onto water. The aqueous phase was extracted with ethyl acetate and then acidified with 2N hydrochloric acid solution. The solid that precipitated was collected by filtration and washed with water to afford a cream-white solid (170 mg). The solid was confirmed as a 50:50 mixture off starting acid and product by ¹H NMR. The solid was used without further purification in the next reaction.

(b) A mixture of the above acetylthiophene derivative (40 mg, 0.164 mmol) and benzothiazole-2-hydrazine (27 mg, 0.164 mmol) in 1 mL of acetic acid was heated at 60° C. for 16 hours. After this time a solid had precipitated which was collected by filtration, washed with water and dried in a vacuum oven to give the title compound as a yellow solid (20 mg, 31%). The ¹H NMR spectrum is summarized in Table 12.

### Example 23

### Preparation of N-acetyl-3-{5-[1-(2-benzothiazolylhydrazono)ethyl]-2-furanyl}-4-chloro-benzenesulfonamide (Compound No. 355 from Table 3)

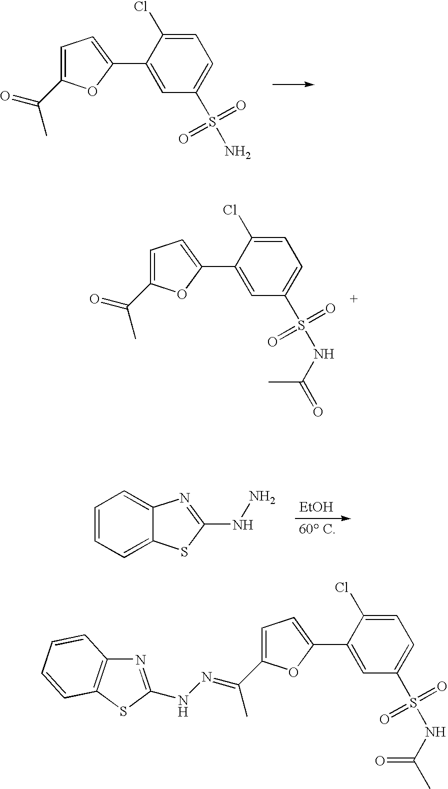

a) 3-(5-acetyl-2-furanyl)-4-chloro-benzenesulfonamide

3-Amino-4-chloro-benzenesulfonamide (200 mg, 1 mmol) was dissolved in a mixture of of concentrated HCl (0.5 mL) and water (5 mL). A solution of sodium nitrite (84 mg, 1.2 mmol) in 1.6 mL of water was then added at 0° C. After 5 minutes, gas evolution started so acetyl furan (110 mg, 1 mmol) in 0.1 mL of acetone was added immediately followed by copper chloride (50 mg, 0.3 mmol) in 0.45 mL of water. After 2 hours, water and ethyl acetate were added to the reaction. The aqueous layer was extracted three times with ethyl acetate. The combined organic phases were washed with water and brine, dried over sodium sulphate and concentrated. Purification on silica gel using petroleum ether/ethyl acetate (40:60 to 70:30) afforded a yellow solid (70 mg, 43%). MS: 300 (M+H). Used without further purification in the next step.

b) Following similar acylation and hydrazone forming conditions to those described in Examples 11, 3-(5-acetyl-2-furanyl)-4-chloro-benzenesulfonamide was converted into

Compound No. 355. ¹H NMR and MS details are summarized in Table 12.

### Example 24

### Preparation of 5-{5-[1-(2-benzothiazolylhydrazono)ethyl]-2-furanyl}-benzene sulfonic acid (Compound No. 387 from Table 3)

a) 3-(5-Acetyl-furan-2-yl)-benzenesulfonic acid

3-Aminobenzenesulfonic acid (1.7 g, 10 mmol) was dissolved in a mixture of water (15 mL) and concentrated hydrochloric acid (5 mL) and the solution was cooled to 5° C. A solution of sodium nitrite (0.8 g, 10 mmol) in water (4 mL) was added slowly with stirring so that the temperature did not rise above 5° C. After about 15 minutes a solution of 2-acetylfuran (1.1 g, 10 mmol) in acetone (8 mL) and a solution of CuCl₂.2H₂0 (0.56 g, 3 mmol) in water (5 mL) were added slowly and simultaneously to the ice cold diazonium salt solution. The reaction mixture was allowed to warm to room temperature and left to stand for 24 hours. Acetone was removed under reduced pressure and the remaining solution was extracted with dichloromethane to remove any starting ketone. The aqueous layer was allowed to evaporate to dryness and the dark green-brown residue was triturated with ethanol (2×25 mL) and the combined ethanol extracts were evaporated and applied to a column of silica gel (40 g). Elution with chloroform:methanol (20:1) gave the product as a dark oil (400 mg) and the first compound to be eluted, Mass spectrum (ESI⁻): 265 (M−1).

b) Benzothiazole-2-hydrazine (35 mg) was added to a solution of 3-(5-acetyl-furan-2-yl)-benzenesulfonic acid (60 mg) in hot ethanol (3 mL). The solution was stirred and heated at 80° C. and a precipitate was observed to form after a few minutes. After 1 hour at reflux the reaction mixture was allowed to cool and the orange precipitate was collected by filtration to give the title hydrazone (50 mg). The ¹H NMR spectrum is summarized in Table 12.

### Example 25

### Preparation of N-pyrimidin-2-yl-3-[5-[1-(2-benzothiazolyl-hydrazono)ethyl]-2-furanyl]-benzenesulfonamide (Compound No. 389 from Table 3)

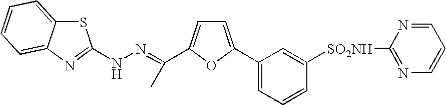

a) N-(pyrimid-2-yl)-3-(5-acetyl-furan-2-yl)-benzenesulfonamide

To a solution of 3-(5-acetyl-furan-2-yl)-benzenesulfonamide (120 mg) (Example 11) and 2-chloropyrimidine (80 mg) in dimethylformamide (1.2 mL) was added anhydrous potassium carbonate (100 mg) and the mixture was heated at 100° C. with stirring for 24 hours. The DMF was removed under reduced pressure and the residue was neutralised with dilute hydrochloric acid and extracted with ethyl acetate (2×25mL). The ethyl acetate layer was dried, evaporated to dryness and the residue was purified by chromatography on silica gel (10 g, CHCl₃) to give the pyrimidyl-sulfonamide as a pale yellow solid (45 mg).

b) Benzothiazole-2-hydrazine (20 mg) was added to a suspension of N-(pyrimidin-2-yl)-3-(5-acetyl-furan-2-yl)-benzenesulfonamide (30 mg) in hot ethanol (3 mL). The suspension was stirred and heated at 80° C. and after 20 minutes a clear solution formed. After 1 hour the solution was allowed to cool and after 2 days an orange precipitate was collected by filtration to give the title hydrazone (26 mg). The ¹H NMR and MS details are summarized in Table 12.

### Example 26

The sulfonamide Compounds No. 388 and 390 were prepared from 3-(5-acetyl-furan-2-yl)-benzenesulfonamide and the appropriate chlorobenzene or chloro-heterocycle, followed by hydrazone formation, using similar conditions to those described in Example 25. The compounds were characterized primarily by their ¹H NMR spectra, a summary of which in provided in Table 12.

### Example 27

### Preparation of 6-{3-[1-(2-benzothiazolylhydrazono)ethyl]-phenyl]-pyridine-2-carboxylic acid (Compound No. 600 from Table 6)

a) Suzuki Coupling

3-acetylphenylboronic acid (802 mg, 4.89 mmol), 6-bromopicolinic acid (764 mg, 3.80 mmol), tetrabutylammoniumbromide (111 mg, 0.38 mmol), potassium carbonate (1.42 g, 10.3 mmol) and trans-dichlorobis(triphenylphosphine)palladium(II) (134 mg, 0.19 mmol) were dissolved in a mixture of dioxane (15 mL) and water (8 mL). Under microwave conditions (120 W) the solution was heated at 100° C. for 1 hour. After cooling the reaction mixture was poured into a water/ethyl acetate mixture. After extraction, the aqueous layer was acidified with 2N hydrochloric acid (pH ˜3) to give a white precipitate which was collected by filtration to give the desired product (731 mg, 80%). ¹H-NMR (d₆DMSO) δ (ppm):2.67 (s, 3H); 7.66 (t, 1H); 8.05 (t, 1H); 8.11 (t, 1H); 8.30 (dd, 1H); 8.44 (d, 1H); 8.69 (s, 1H).

b) Hydrazone Formation

The product from part a) (100 mg, 0.41 mmol) was dissolved in hot ethanol (2 mL) and 2-hydrazinobenzothiazole (68 mg, 0.41 mmol) in ethanol (2 mL) was added. The clear solution was stirred and heated at 60° C. for 14 hours and during this time a pale cream precipitate formed. The reaction was allowed to cool and then filtered to give the hydrazone product as a cream solid (143 mg, 90%). The ¹H NMR and MS details are summarized in Table 12.

### Example 28

### Preparation of Ethyl 6-{3-[1-(2-benzothiazolylhydrazono)ethyl]-phenyl]-pyridine-2-carboxylate (Compound No. 602 from Table 6)

a) Esterification

The product from Example 27, part a) (50 mg, 0.21 mmol) was dissolved in 1.3 mL ethanol and thionylchloride (11 μL, 0.15 mmol) was added. The clear solution was stirred and heated to reflux for 5 hours. The reaction was allowed to cool and then concentrated whereafter a mixture of ethyl acetate and 2N sodium hydroxide were added. The organic layer was extracted with ethyl acetate (2×15 mL) and the combined organic layers were washed with brine, dried (Na₂SO₄) and concentrated to afford the desired intermediate (53 mg, 94%). ¹H NMR (d-chloroform) δ (ppm): 1.49 (t, 3H); 2.71 (s, 3H); 4.52 (q, 2H); 7.62 (t, 1H); 7.92-8.02 (m, 2H), 8.05 (dd, 1H); 8.11 (dd, 1H); 8.37 (dd, 1H), 8.64 (s, 1H).

b) Hydrazone Formation

The ester from part a) (16 mg, 0.059 mmol) was dissolved in hot ethanol (1 mL) and 2-hydrazinobenzothiazole (10 mg, 0.059 mmol) in ethanol (1 mL) was added. The clear solution was stirred and heated at 60° C. for 14 hours and during this time a pale cream precipitate formed. The reaction was allowed to cool and then filtered to give the hydrazone product as a cream solid (19 mg, 77%). The ¹H NMR and MS details are summarized in Table 12.

### Example 29

The ester Compounds No. 603-605 and 624 were prepared in a similar fashion to that described for Compound 602 in Example 28 above. The ¹H NMR spectrum or MS details are summarized in Table 12.

### Example 30

The acylsulfonamide Compounds No. 601, 608 and 617 were prepared in a similar fashion to that described for Compound 301 in Example 8 above. The MS details are summarized in Table 12.

### Example 31

### Preparation of 3-[1-(2-benzothiazolylhydrazono)indan-6-yl]-benzoic acid (Compound No. 606 from Table 6)

a) Suzuki Coupling

3-carboxyphenylboronic acid (154 mg, 0.93 mmol), 6-bromo-1-indanone (198 mg, 0.94 mmol), tetrabutylammoniumbromide (28 mg, 0.093 mmol), potassium carbonate (354 mg, 2.57 mmol) and trans-dichlorobis(triphenylphosphine)palladium(II) (33 mg, 0.047 mmol) were dissolved in a mixture of 4 mL of dioxane and 2 mL water. Under microwave conditions (120 W) the solution was heated at 100° C. for 1 hour. After cooling, the reaction mixture was poured into a water/ethyl acetate mixture. After extraction, the aqueous layer was acidified with 2N hydrochloric acid (pH ˜3) to give a white precipitate which was collected by filtration to give the desired product (103 mg, 44%). ¹H NMR (d₆DMSO) δ (ppm): 2.71(m, 2H); 3.16 (m, 2H); 7.62 (t, 1H); 7.71 (d, 1H); 7.87 (s, 1H); 7.95-8.05 (m, 3H); 8.21 (s, 1H).

b) Hydrazone Formation

The product from part a) (50 mg, 0.20 mmol) was dissolved in hot ethanol (2 mL) and 2-hydrazinobenzothiazole (33 mg, 0.20 mmol) in ethanol (2 mL) was added. The clear solution was stirred and heated at 60° C. for 14 hours and during this time a pale cream precipitate formed. The reaction was allowed to cool and then filtered to give the hydrazone product, Compound No. 606 as a cream solid (66 mg, 83%). The ¹H NMR spectrum is summarized in Table 12.

### Example 32

### N-Benzothiazol-2-yl-N′-(1-{3-[6-(1H-tetrazol-5-yl)-pyridin-2-yl]-phenyl}-ethylidene)-hydrazine (Compound No. 607)

a) 6-(3-Acetyl-phenyl)-pyridine-2-carbonitrile

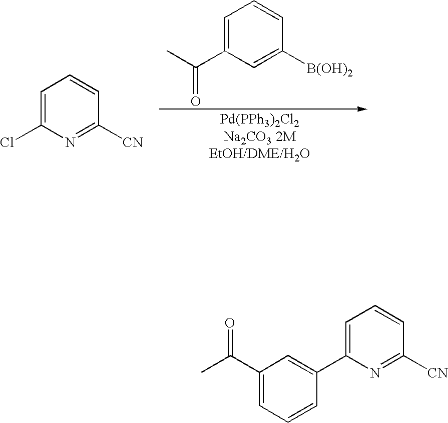

2-Chloro-6-cyanopyridine (100 mg, 0.72 mmol), 3-acetylphenylboronic acid (138 mg, 0.86 mmol), Pd(PPh₃)₂Cl₂ (35 mg) and sodium carbonate (0.66 mL of a 2M aqueous solution) in 6 mL of a 1:1:1 EtOH/DME/H₂O mixture were heated at 80° C. for 3 hours. A light brown solid precipitated. The whole reaction was poured onto water and ethyl acetate. The aqueous layer was extracted three times with AcOEt. The combined organic layers were washed with water and brine, dried over Na₂SO₄ and concentrated. The solid recovered was triturated with Et₂O and dried. A white solid was obtained (93 mg, 58%). ¹H NMR (d₆-DMSO, ppm): 2.68 (s, 3H), 7.71 (t, 1H), 8.05 (dd, 1H), 8.10 (dt, 1H), 8.20 (t, 1H), 8.36 (dt, 1H), 8.43 (dd, 1H), 8.62 (t, 1H). ¹³C NMR (d₆-DMSO, ppm): 26.9, 117.5, 124.8, 126.3, 128.0, 129.5, 129.7, 131.3, 132.7, 137.2, 137.5, 139.3, 156.8, 197.7.

b) 1-{3-[6-(1H-Tetrazol-5-yl)-pyridin-2-yl]-phenyl}-ethanone

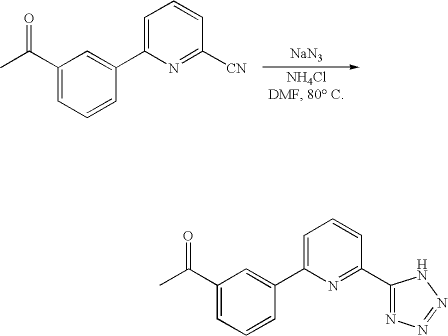

6-(3-Acetyl-phenyl)-pyridine-2-carbonitrile (44 mg, 0.2 mmol), sodium azide (19 mg, 0.22 mmol) and ammonium chloride (15.2 mg, 0.22 mmol) were heated at 80° C. in 1 mL of DMF. After 3 hours, TLC (90:10 CH₂Cl₂/MeOH) showed complete conversion of starting material. The reaction was cooled down and concentrated in vacuo. The residue was taken into water and HCl 2N was added. The precipitate was collected by filtration and dried (25 mg, 47%). LCMS: M+H=266.

c) Hydrazone Formation

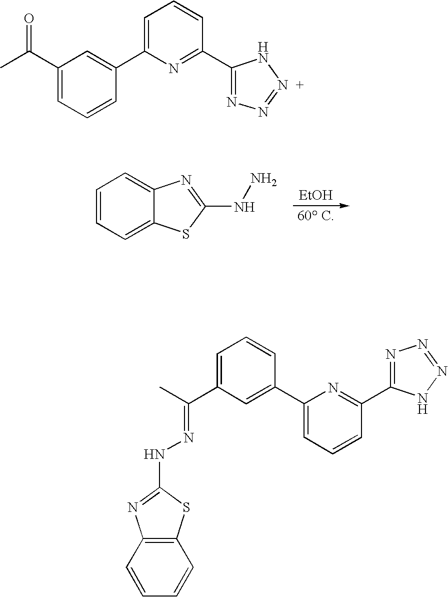

1-{3-[6-(1H-Tetrazol-5-yl)-pyridin-2-yl]-phenyl}-ethanone (30mg, 0.11 mmol) and benzothiazol-2-yl-hydrazine (21 mg, 0.17 mmol) were heated at 60° C. in ethanol. After 2 hours a white solid had formed. It was collected by filtration and rinsed with water and a small amount of diethyl ether. The solid was then dried in vacuum (18 mg, 38%). ¹H NMR and MS details are listed in Table 12.

### Example 33

### 6-{3-[1-(2-Benzothiazolylhydrazono)-ethyl]-phenyl}-pyridine-2-carboxylic acid (1H-tetrazol-5-yl)-amide (Compound No. 609)

a) 6-(3-Acetyl-phenyl)-pyridine-2-carboxylic acid (1H-tetrazol-5-yl)-amide

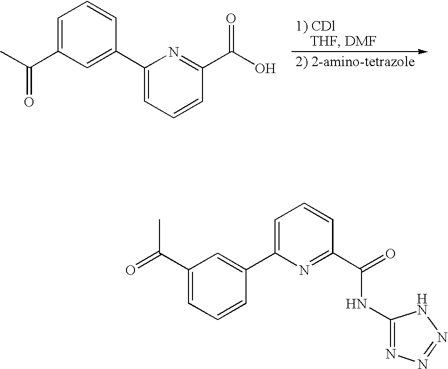

1,1′-Carbonyldiimidazole (170 mg, 1.05 mmol) was added to a solution of 6-(3-acetyl-phenyl)-pyridine-2-carboxylic acid (241 mg, 1 mmol) in THF/DMF (2.4 mL/1.2 mL). The reaction was stirred at room temperature for 1.5 hours. 2-aminotetrazole was then added and the reaction was heated at 60° C. for 2 hours. The reaction mixture was concentrated under reduced pressure and water was added, followed by 2M HCl. A white solid precipitated and was collected by filtration, rinsed with water and dried (243 mg, 79%). ¹H NMR (d₆-DMSO, ppm): 4.77 (s, 3H), 7.72 (t, 1H), 8.08 (d, 1H), 8.17-8.27 (m, 2H), 8.42 (d, 1H), 8.3 (m, 2H).

b) Hydrazone Formation

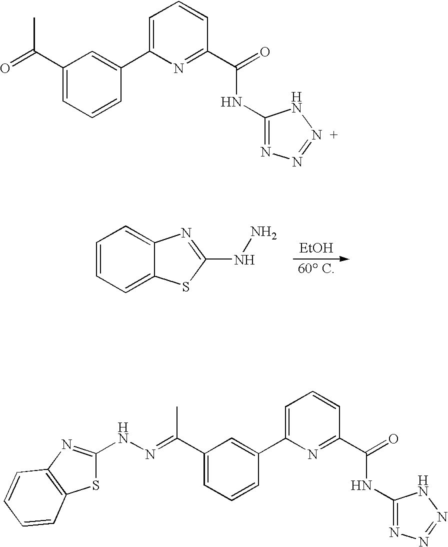

6-(3-Acetyl-phenyl)-pyridine-2-carboxylic acid (1H-tetrazol-5-yl)-amide (154 mg, 0.5 mmol) and benzothiazol-2-yl-hydrazine (91 mg, 0.55 mmol) were heated at 60° C. in ethanol. After 4 hours a white solid had formed which was collected by filtration and rinsed with water and a small amount of diethyl ether. The solid was then dried in vacuum (139 mg, 61%). ¹H NMR and MS details are listed in Table 12.

### Example 34

### Preparation of 6-[1-(2-benzothiazolylhydrazono)indan-6-yl]pyridine-2-carboxylic acid (Compound No. 610 from Table 6)

a) Preparation of Boronic Ester

6-bromo-1-indanone (183 mg, 0.87 mmol), bis(pinacolato)diboron (286 mg, 1.12 mmol), potassium acetate (186 mg, 1.72 mmol) and [1,1′-bis(diphenylphosphino)ferrocene]-dichloropalladium (16 mg, 0.022 mmol) were dissolved in 4 mL methanol and the solution was stirred at 60° C. for 14 hours. The reaction was allowed to cool, filtered over celite and concentrated to give a dark oil. The crude product was purified by flash chromatography on silica, using a 7:3 and then 9:1 ratio of petroleum hydrocarbons and ethyl acetate as eluent to give the boronic ester (50 mg, 22%). ¹H NMR (d-chloroform) δ (ppm):1.36 (s, 12H); 2.69 (m, 2H); 3.16 (m, 2H); 7.48 (d, 1H); 8.00 (s, 1H), 8.25 (s, 1H)

b) Suzuki Coupling

The boronic acid from part a) (50 mg, 0.19 mmol), 6-bromopicolinic acid (34 mg, 0.17 mmol), tetrabutylammoniumborohydride (5 mg, 0.017 mmol), potassium carbonate (63 mg, 0.46 mmol) and trans-dichlorobis(triphenylphosphine)palladium(II) (6 mg, 0.0085 mmol) were dissolved in 1 mL dioxane and 0.5 mL water. Under microwave conditions (120 W) the solution was heated at 100° C. for 1 hour. After cooling, the reaction mixture was poured into a water/ethyl acetate mixture. After extraction, the aqueous layer was acidified with 2N hydrochloric acid (pH ˜3) and extracted with ethyl acetate (2×20 mL), The combined organic layers were washed with brine, dried (Na₂SO₄) and concentrated to give the desired product (25 mg, 60%). ¹H NMR (d-chloroform) δ (ppm): 2.80 (m, 2H); 3.24 (m, 2H); 7.67 (d, 1H); 7.78 (dd, 1H); 8.07 (d, 1H); 8.21 (t, 1H); 8.32 (dd, 1H), 8.37 (s, 1H).

Hydrazone Formation

The product from part b) (25 mg, 0099 mmol) was dissolved in hot ethanol (1 mL) and 2-hydrazinobenzothiazole (25 mg, 0.15 mmol) in ethanol (1 mL) was added. The clear solution was stirred and heated at 60° C. for 14 hours and during this time a pale cream precipitate formed. The reaction was allowed to cool and then filtered to give the crude hydrazone product which was purified by washing with methanol to give the pure hydrazone product as a cream solid (16 mg, 40%). ¹H NMR and MS details are summarized in Table 12.

### Example 35

### Preparation of 6-{5-[1-(2-benzothiazolylhydrazono)ethyl]-2-thienyl]pyridine-2-carboxylic acid (Compound No. 502 from Table 5)

Compound No. 502 was prepared following essentially the same steps as used for Compound 610 above, starting from commercially available 2-acetyl-5-bromothiophene. The ¹H NMR and MS details are summarized in Table 12.

### Example 36

### Preparation of 6-[8-(2-benzothiazolylhydrazono)-5,6,7,8-tetrahydronaphthalen-2-yl]-pyridine-2-carboxylic acid (Compound No. 611 in Table 6)

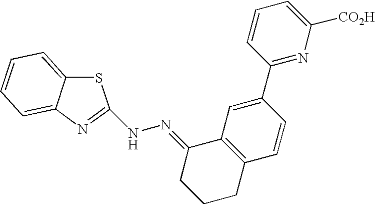

a) Preparation of 1-tetralone-7-boronic ester

7-Bromo-tetralone (200 mg, 0.89 mmol), bis(pinacolato)diboron (296 mg, 1.16 mmol), Potassium acetate (192 mg, 1.78 mmol) and [1,1′-bis(diphenylphosphino)ferrocene]-dichloropalladium (17 mg, 0.023 mmol) were dissolved in 4 mL methanol and the solution was stirred at 60° C. for 14 hours. The reaction was allowed to cool, filtered over celite and concentrated to give a dark oil. The crude product was purified by flash chromatography on silica, using petroleum benzine and then 9:1 ratio of petroleum benzine and ethyl acetate as eluent to give the boronic ester (160 mg, 66%). ¹H NMR (d-chloroform) δ (ppm): 1.31 (s, 12H); 2.11 (m, 2H); 2.63 (t, 2H); 2.95 (t, 2H), 7.21 (s, 1H), 7.85 (dd, 1H), 8.47 (s, 1H).

b) Suzuki Coupling

The boronic ester from part a) (80 mg, 0.29 mmol), 6-bromopicolinic acid (51 mg, 0.26 mmol), tetrabutylammoniumbromide (7.5 mg, 0.026 mmol), potassium carbonate (94 mg, 0.69 mmol) and trans-dichlorobis(triphenylphosphine)palladium(II) (8.7 mg, 0.013 mmol) were dissolved in a mixture of 1 mL dioxane and 0.5 mL water. Under microwave conditions (120 W) the solution was heated at 100° C. for 1 hour. After cooling the reaction mixture was poured into a water/ethyl acetate mixture. After extraction, the aqueous layer was acidified with 2N hydrochloric acid (pH ˜3) and extracted with ethyl acetate (2×20 mL), The combined organic layers were washed with brine, dried (Na₂SO₄) and concentrated to give the desired product (30 mg, 39%). ¹H NMR (d₆-dmso) δ (ppm): 2.06 (m, 2H); 2.63 (t, 2H); 2.99 (t, 2H); 7.96 (dd, 1H); 8.03 (t, 1H); 8.18 (dd, 1H); 8.31 (dd, 1H), 8.59 (d, 1H).

c) Hydrazone Formation

The tetralone derivative from part b) (34 mg, 0.13 mmol) was dissolved in hot ethanol (1 mL) and 2-hydrazinobenzothiazole (32 mg, 0.19 mmol) in ethanol (1 mL) was added. The clear solution was stirred and heated at 60° C. for 6 hours and during this time a pale cream precipitate formed. The reaction was allowed to cool and then filtered and washed with hot methanol to give the hydrazone product as a cream solid (22 mg, 41%). ¹H NMR (d₆-dmso) δ (ppm): 1.90 (m, 2H); 2.83 (m, 4H); 7.10 (t, 1H); 7.28 (t, 1H); 7.37 (d, 2H); 7.75 (d, 1H); 8.02 (dd, 1H); 8.05-8.18 (m, 3H), 8.81 (s, 1H), MS 415 (M+1).

### Example 37

### Preparation of 6-[4-(2-benzothiazolylhydrazono)-chroman-4-on-6-yl]-pyridine-2-carboxylic acid (compound 612 from Table 6)

Compound No. 612 was prepared following essentially the same steps as used for Compound 611 above and starting from commercially available 6-bromochroman-4-one. The ¹H NMR and MS details are summarized in Table 12.

### Example 38

### Preparation of 5-{5-[1-(2-benzothiazolylhydrazono)ethyl]-2-thiophenyl}-furan-2-carboxylic acid (Compound No. 700 from Table 7)

a) 5-(5-Acetyl-thiophen-2-yl)-furan-2-carboxylic acid

A mixture of 5-bromo-furoic acid (100 mg, 0.52 mmol), 2-acetyl-thiophene-5-boronic acid (125 mg, 0.68 mmol), PdCl₂(PPh₃)₂ (18 mg, 5 mol %), tetrabutylammonium bromide (17 mg, 0.07 mmol), potassium carbonate (578 mg, 1.56 mmol) in a dioxane:H₂O (2:1 ml) mixture was heated at 70° C. under microwave irradiation for 30 minutes. The reaction was cooled, diluted with ethyl acetate and poured onto water. The aqueous phase was extracted with ethyl acetate and then acidified with 2N hydrochloric acid solution. The solid that precipitated was filtered off and washed with water to afford an off-white solid (58 mg, 46%). ¹H NMR (d₆-DMSO) δ: 7.96 (1H, d, J=4.2 Hz), 7.65 (1H, d, J=3.9 Hz), 7.35 (1H, d, J=3.6 Hz), 7.20 (1H, d, J=3.6 Hz), 2.56 (3H, s).

b) Hydrazone Formation

A mixture of the furoic acid from part a) (20 mg, 0.085 mmol) and benzothiazole-2-hydrazine (15 mg, 0.094 mmol) in 1 mL of acetic acid was heated at 60° C. for 16 hours. After this time a solid had precipitated. It was collected by filtration, washed with water and dried in a vacuum oven to give the title compound as a pale brown solid (25 mg, 77%). The ¹H NMR spectrum is summarized in Table 12.

### Example 39

### Preparation of 5-{5-[1-(2-benzothiazolylhydrazono)ethyl]-2-thiophenyl}-thiophene-2-carboxylic acid (Compound No. 701 from Table 7)

a) 5-(5-Acetyl-thiophen-2-yl)-thiophene-2-carboxylic acid

A mixture of 5-bromo-2-thiophene carboxylic acid (325 mg, 1.57 mmol), 2-acetyl-thiophene-5-boronic acid (373 mg, 2.2 mmol), PdCl₂(PPh₃)₂ (55 mg, 5 mol %), tetrabutylammonium bromide (51 mg, 0.16 mmol), potassium carbonate (578 mg, 4.71 mmol) in a dioxane:H₂O (5:2.5 mL) mixture was heated at 70° C. under microwave irradiation for 30 minutes. The reaction was cooled down, diluted with ethyl acetate and poured onto water. The aqueous phase was extracted with ethyl acetate and then acidified with 2N hydrochloric acid solution. The solid that precipitated was filtered off at the pump and washed with water to afford a pale brown solid (160 mg, 41%). ¹H NMR (d₆-DMSO) δ: 7.93 (1H, d, J=4.2 Hz), 7.70 (1H, d, J=3.9 Hz), 7.59 (1H, d, J=3.9 Hz), 7.55 (1H, d, J=3.9 Hz), 2.54 (3H, s).

b) Hydrazone Formation

A mixture of the thiophene from part a) (20 mg, 0.079 mmol) and benzothiazole-2-hydrazine (15 mg, 0.087 mmol) in 1 mL of acetic acid was heated at 60° C. for 16 hours. After this time a solid had precipitated. It was collected by filtration, washed with water and dried in a vacuum oven to give the title compound as a pale brown solid (27 mg, 84%). The ¹H NMR spectrum is summarized in Table 12.

### Example 40

### N-Acetyl-6-[8-(Benzothiazol-2-yl-hydrazono)-5,6,7,8-tetrahydro-naphthalen-2-yl]-pyridine-2-sulfonamide (Compound No. 615 from Table 6)

a) 6-Bromo-pyridine-2-(N-tert-butyl)-sulfonamide

Benzylmercaptan (6.3 g, 51 mmol) was added to sodium hydroxide (13.6 g, 15% aqueous solution (w/w)). Toluene (15 mL) and then 2,6-dibromopyridine (5 g, 34 mmol) and finally tetrabutylammonium bromide (300 mg, 0.93 mmol) were added. The reaction was heated at 80° C. for 4 h and the mixture was then decanted and the aqueous layer separated. The organic layer was washed with water and dried over MgSO₄. The toluene solution was concentrated to afford a thick yellow oil which was used in the next step without further purification. A 5% aqueous solution of sodium hypochlorite (150 mL) was added dropwise to the 2-bromo-6-benzylthiopyridine (6.71 g, 10 mmol) in a mixture of water (80 mL), CH₂Cl₂ (103 mL) and concentrated hydrochloric acid (13 mL) at 0° C. After the addition was completed, the reaction was stirred at 0° C. for a further 30 minutes. The reaction was then diluted with ice cold water and extracted twice with ice cold CH₂Cl₂. The combined organic layers were washed with brine and dried over Na₂SO₄. The filtrate was cooled to −78° C. and t-butylamine (13 mL, 124 mmol) was added. The reaction was slowly warmed to room temperature and the reaction was then acidified to pH=1 with concentrated hydrochloric acid and extracted three times with CH₂Cl₂. The combined organic layers were washed with water and brine, dried over Na₂SO₄ and concentrated. The solid residue was purified by flash chromatography on SiO₂ with flashmaster purification system. Conditions: Petroleum ether/ethyl acetate 80:20 for 2 minutes, 80:20 to 65:35 in 15 minutes, 65:35 for 10 minutes and 65:35 to 50:50 in 3 minutes. A brown solid was obtained. It was finally triturated with Et₂O to afford a white solid (1.51 g, 22%). NMR ¹H (d₆-DMSO, ppm): 8.00-7.93 (m, 2H), 7.87-7.84 (m, 1H), 7.85 (br s., 1H), 1.09 (s, 9H). NMR ¹³C NMR (d₆-DMSO, ppm): 160.5, 141.7, 141.0, 131.1, 120.9, 53.6, 29.8.

b) 6-Bromo-pyridine-2-sulfonamide

TFA (2 mL) was added to 6-bromo-pyridine-2-(N-tert-butyl)-sulfonamide (478 mg, 1.63 mmol) and the reaction was stirred at 60° C. for 3 hours. TFA was removed under vacuum and a mixture of Petroleum ether and Et₂O was added leading to the precipitation of a brown solid. It was collected and triturated three times with Et₂O. A white solid was obtained (125 mg, 32%). NMR ¹H (d₆-DMSO, ppm): 7.98 (t, J=7.5 Hz, 1H), 7.91 (dd, J=7.5 and 1.2 Hz, 1H), 7.87 (dd, J=7.5 and 0.9 Hz, 1H).

c) 6-(8-Oxo-5,6,7,8-tetrahydro-naphthalen-2-yl)-pyridine-2-sulfonamide

6-Bromo-pyridine-2-sulfonamide (65 mg, 0.27 mmol), 7-(4,4,5,5-Tetramethyl-[1,3,2]dioxaborolan-2-yl)-3,4-dihydro-2H-naphthalen-1-one (97 mg, 0.36 mmol), tetrabutylammonium bromide (TBAB) (10 mg, 0.04 mmol) and Pd(PPh₃)₂Cl₂ (13 mg, 0.02 mmol) in dioxane:water (1.2 mL/0.6 mL) were heated at 100° C. in a microwave reactor for 1 hour. After this time, water was added and the aqueous phase was extracted three times with ethyl acetate. The combined organic phases were washed with water and brine and dried over Na₂SO₄. Concentration afforded a brown solid which was triturated with Et₂O. An off-white solid was obtained. Analysis (NMR) showed presence of impurities but the compound was on-reacted without further purification. NMR ¹H (d₆-DMSO, ppm): 8.6 (d, J=2.1 Hz, 1H), 8.36 (dd, J=8.1 and 2.1 Hz, 1H), 8.20 (dd, J=9.03 and 1 Hz, 1H), 8.11 (t, J=7.6 Hz, 1H), 7.85 (dd, J=7.6 and 1 Hz, 1H), 7.53-7.50 (m, 3H), 3.01-2.98 (m, 2H), 2.66-2.62 (m, 2H), 2.10-2.04 (m, 2H).

d) N-Acetyl-6-(8-Oxo-5,6,7,8-tetrahydro-naphthalen-2-yl)-pyridine-2-sulfonamide

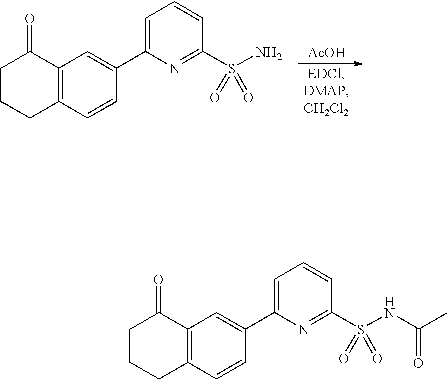

6-(8-Oxo-5,6,7,8-tetrahydro-naphthalen-2-yl)-pyridine-2-sulfonamide (68 mg, 0.23 mmol) EDCI (85.5 mg, 0.45 mmol), DMAP (57.1 mg, 0.45 mmol) and acetic acid (16 μL, 0.27 mmol) were stirred in CH₂Cl₂ at room temperature for 16 h. After this time, more CH₂Cl₂ was added followed by 2M HCl. The organic layer was washed twice with 2M HCl, NaHCO₃ was then added and the organic layer was washed four times with a saturated solution of NaHCO₃. The combined basic aqueous layer was acidified with 2M HCl until precipitation of a white solid at pH=1. The solid was collected by filtration, rinsed with water and dried in a vacuum oven. A white solid was obtained (41 mg, 53%). NMR ¹H (d₆-DMSO, ppm): 8.6 (d, 1H), 8.17 (dd, 1H), 8.11 (t, 1H), 8.03 (dd, 2H), 7.37 (d, 1H), 3.04 (t, 2H), 2.73 (t, 2H), 2.23 (s, 3H), 2.20 (t, 2H). LCMS: M+H=345.

### e) N-Acetyl-6-[8-(benzothiazol-2-yl-hydrazono)-5,6,7,8-tetrahydro-naphthalen-2-yl]-pyridine-2-sulfonamide (Compound No. 615)

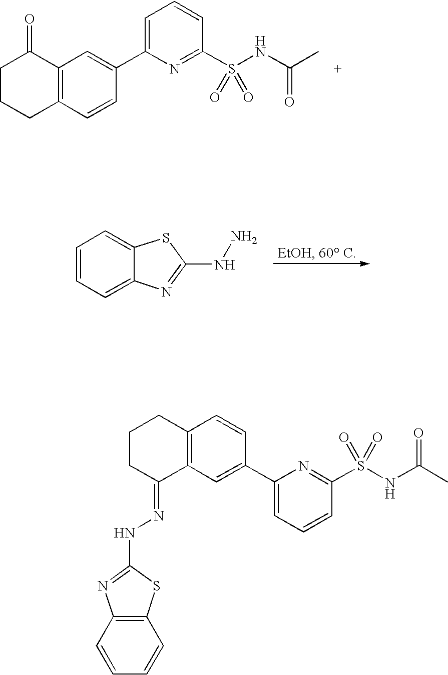

N-Acetyl-6-(8-Oxo-5,6,7,8-tetrahydro-naphthalen-2-yl)-pyridine-2-sulfonamide (25 mg, 0.073 mmol) and benzothiazol-2-yl-hydrazine (12 mg, 0.073 mmol) were stirred in 0.5 mL of EtOH at 60° C. for 16 h. The white solid formed was collected by filtration, rinsed with EtOH and Et₂O and dried in a vacuum oven (22 mg, 61%). NMR and LCMS details are listed in Table 12.

### Example 41

Compound No. 613 was prepared following a similar sequence of reactions to those described for Compound No. 615 in Example 40 above. NMR and LCMS details for the product are listed in Table 12.

### Example 42

Compound No. 614 was prepared following a similar sequence of reactions to those described for Compound No. 606 in Example 31 above. NMR and LCMS details for the product are listed in Table 12.

### Example 43

### 6-[8-(Benzothiazol-2-yl-hydrazono)-5,6,7,8-tetrahydro-naphthalen-2-yl]-pyridine-2-sulfonic acid (6-phenyl-hexanoyl)-amide (Compound No. 616)

a) N-(6-phenyl-hexanoyl)-6-(8-Oxo-5,6,7,8-tetrahydro-naphthalen-2-yl)-pyridine-2-sulfonamide

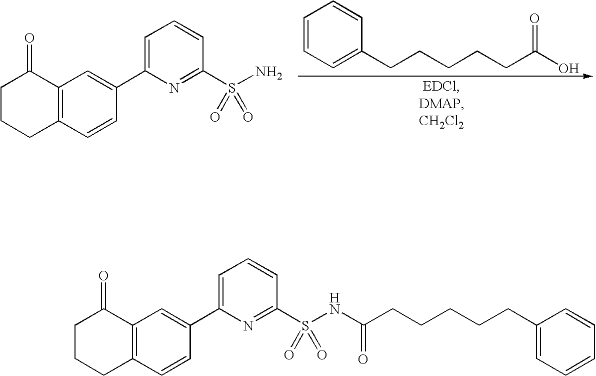

6-(8-Oxo-5,6,7,8-tetrahydro-naphthalen-2-yl)-pyridine-2-sulfonamide (11 mg, 36.4 μmol), EDCI (17 mg, 72 μmol), DMAP (11.1 mg, 72 μmol) and 6-phenylhexanoic acid (8.4 mg, 44 μmol) were stirred in CH₂Cl₂ at room temperature for 16 h. CH₂Cl₂ was added, followed by 2M HCl and the organic layer was washed twice with 2M HCl. The organic layer was then washed with water and brine, dried over Na₂SO₄ and concentrated. The residue obtained was purified by flash chromatography on SiO₂ using CH₂Cl₂/MeOH (100:0 then 99:1). A colourless film was obtained (13 mg, 75%). NMR ¹H (CDCl₃, ppm): 8.78 (br s, 1H), 8.57 (d, 1H), 8.16 (dd, 1H), 8.12 (t, 1H), 7.99 (dd, 2H), 7.30 (d, 1H), 7.23-7.11 (m, 3H), 7.05-7.02 (m, 2H), 2.98-2.94 (m, 2H), 2.68-2.63 (m, 2H), 2.43-2.33 (m, 4H), 2.12-2.08 (m, 2H), 1.57-1.52 (m, 2H), 1.47-1.42 (m, 2H), 1.26-1.19 (m, 2H). LCMS: M−H=475.3.

b) 6-[8-(Benzothiazol-2-yl-hydrazono)-5,6,7,8-tetrahydro-naphthalen-2-yl]-pyridine-2-sulfonic acid (6-phenyl-hexanoyl)-amide (Compound No 616)

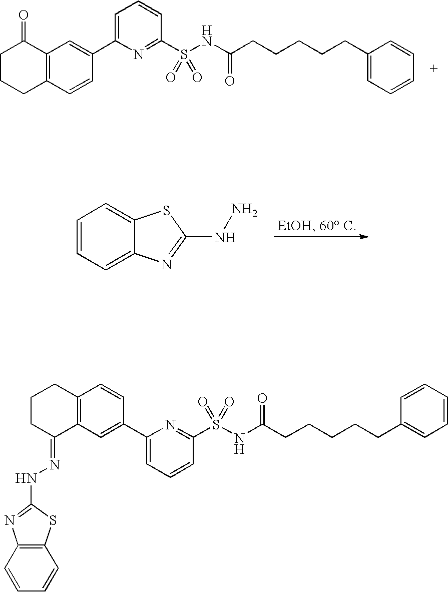

N-(6-phenyl-hexanoyl)-6-(8-Oxo-5,6,7,8-tetrahydro-naphthalen-2-yl)-pyridine-2-sulfonamide (13 mg, 0.027 mmol) and benzothiazol-2-yl-hydrazine (5 mg, 0.027 mmol) were stirred in 0.25 mL of EtOH at 60° C. for 16 h. After addition of water, the solid product was collected by filtration, rinsed with EtOH and Et₂O and dried in a vacuum oven to give the hydrazone as a pale yellow solid (11 mg, 65%). NMR and LCMS details are provided in Table 12.

### Example 44

### N-{6-[8-(Benzothiazol-2-yl-hydrazono)-5,6,7,8-tetrahydro-naphthalen-2-yl]-pyridin-2-yl}-4-methyl-benzenesulfonamide (Compound No 618)

a) N-(6-Bromo-pyridin-2-yl)-4-methyl-benzenesulfonamide

To a solution of 2-amino-6-bromopyridine (500 mg, 2.89 mmol) in anhydrous dichloromethane (10 mL) at 0° C., DIPEA (757 μL, 4.34 mmol) was added, followed by 4-toluenesulfonylchloride (551 mg, 2.89 mmol). The reaction mixture was stirred for 18 hours at ambient temperature. The layers were separated between saturated aqueous sodium bicarbonate and dichloromethane. The organic layer was washed with brine, dried and concentrated, followed by purification by column chromatography (2:1 petroleum ether/ethyl acetate). Isolated 502 mg (53%) of clean product.

b) N-[6-(8-oxo-5,6,7,8-tetrahydronaphthanenyl-pyridin-2-yl)-4-methyl-benzenesulfonamide

To a mixture of the bromopyridine from part a) above (100 mg, 0.305 mmol) in dioxane (1.3 mL) and water (0.7 mL), 4-tetralone-6-boronic acid pinacol ester (108 mg, 0.397 mol) was added, followed by TBAB (10 mg), K₂CO₃ (127 mg) and Pd(PPh₃)₂Cl₂ (12 mg). The mixture was reacted in a microwave for 60 minutes at 100° C.; 150 W. The contents were concentrated, separated between water and dichloromethane. The organic layer was washed with brine, dried and the removed solvent to give 77 mg (65%) of clean product.

c) Hydrazone Formation

The above product (75 mg, 0.191 mmol) in ethanol (2 mL) and 2-benzothiazole hydrazine (32 mg, 0.191 mmol) was heated at 65° C. for 18 hours. The product was isolated cleanly (64 mg, 62%) by cooling and filtration. LCMS details for the product are in Table 12.

### Example 45

Compound No.'s 619-623 were prepared using the appropriate N-pyridyl-sulfonamide following a similar sequence of reactions to those described for Compound No. 618 in Example 44 above. NMR and/or LCMS details for the products are listed in Table 12.

### Example 46

### 6-[4-(Benzothiazol-2-yl-hydrazono)-1-ethyl-1,2,3,4-tetrahydro-quinolinyl-6-yl]-pyridine-2-carboxylic acid (Compound No 625)

a) 1-Ethyl-6-iodo-4-oxo-1,4-dihydroquinoline-3-carboxylic acid

1-Ethyl-6-iodo-4-oxo-1,4-dihydroquinoline-3-carboxylic acid ethyl ester was prepared following literature procedures (WO 2006/120545). The ethyl ester (931 mg, 2.52 mmol) was stirred in 1 N NaOH (21 mL) and MeOH (10 mL) at 90° C. for 3 h. After concentration, the mixture was taken up into water (100 mL) and acidified with 10% citric acid solution. The resulting precipitate was collected by filtration to afford the carboxylic acid (802 mg, 93%). ¹H NMR (DMSO-d6) δ (ppm): 1.36 (t, 3H), 4.54 (q, 2H), 7.81 (d, 1H), 8.18 (dd, 1H), 8.58 (s, 1H), 9.03 (s, 1H).

b) 1-Ethyl-6-iodo-4-oxo-1,2,3,4-tetrahydroquinoline

Sodiumborohydride (243 mg, 6.46 mmol) was added portionwise to a solution of the carboxylic acid from part a) (500 mg, 1.46 mmol) in methanol (20 mL) at room temperature. Once addition was complete, p-toluenesulfonic acid monohydrate (30 mg, 0.15 mmol) was added and the reaction mixture was stirred at 65° C. for 1 hour after which the mixture was concentrated. The residue was taken up into ethylacetate (50 mL) and washed with saturated sodium bicarbonate, water and brine respectively. Drying (Na₂SO₄) and concentration afforded the reduced and decarboxylated compound (297 mg, 68%). ¹H NMR (d-chloroform) δ (ppm): 1.18 (t, 3H); 2.65 (t, 2H); 3.37-3.50 (m, 4H); 6.50 (d, 1H); 7.55 (dd, 1H); 8.13 (s, 1H).

c) The iodo compound from part b) above was converted through to final Compound No. 625 in three steps using similar conditions to those described in Example 36, parts a) to c). Thus preparation of the boronic acid pinacol ester was followed by Suzuki coupling with 6-bromopicolinic acid and then benzothiazole-2-hydrazone formation. NMR details for the final compound are provided in Table 12.

### Example 47

### 6-[4-(Benzothiazol-2-yl-hydrazono)-1,2,3,4-tetrahydro-quinolinyl-6-yl]pyridine-2-carboxylic acid (Compound No 626)

a) 6-Bromo-2,3-dihydro-1H-quinolin-4-one

4-Bromoaniline (2.0 g, 11.6 mmol) and acrylic acid (0.95 mL, 13.9 mmol) were stirred in toluene (15 mL) at 100° C. for 3 days. After cooling, the reaction mixture was extracted with 1N NaOH (150 mL). The aqueous layer was acidified with 2N HCl (pH˜3) and subsequently extracted with ethylacetate (2×100 mL). The combined organic layers were washed with brine, dried (Na₂SO₄) and concentrated to afford N-(4-bromophenyl)-3-aminopropionic acid (1.65 g, 58%). A mixture of the carboxylic acid (1.64 g, 6.72 mmol) in polyphosphoric acid (30 g) was stirred at 90° C. overnight. The reaction was allowed to cool and ice-water was added. The mixture was then extracted with ethylacetate (2×200 mL). The combined organic layers were washed with 1N NaOH, water and brine respectively. Drying (Na₂SO₄) and concentration afforded the cyclized product (0.88 g, 58%). ¹H NMR (d-chloroform) δ (ppm): 2.67 (t, 2H), 3.54 (t, 2H), 6.57 (d, 1H), 7.33 (dd, 1H), 7.92 (s, 1H).

b) The bromoquinoline from part a) above was converted through to final Compound No. 626 in three steps using similar conditions to those described in Example 36, parts a) to c). Thus preparation of the boronic acid pinacol ester was followed by Suzuki coupling with 6-bromopicolinic acid and then benzothiazole-2-hydrazone formation. NMR details for the final compound are provided in Table 12.

### Example 48

### 6-[4-(Benzothiazol-2-yl-hydrazono)-2,3-dihydro-benzothiopyran-6-yl]-pyridine-2-carboxylic acid (Compound No 627)

a) 6-Bromo-thiochroman-4-one

A mixture of 4-bromobenzenethiol (1.55 g, 8.2 mmol) in 2N NaOH (4 mL) was heated to refluxing temperature. An ice cold solution of 3-chloropropionic acid (1.78 g, 16.5 mmol) in 2N NaOH (8 mL) was added dropwise. Subsequently, the mixture was refluxed for 1 h. The reaction mixture was cooled and washed with ethylacetate (100 mL). The aqueous layer was acidified, using 2N HCl (pH˜3) and extracted with ethylacetate (2×50 mL). The combined organic layers were extracted with sat. sodium bicarbonate solution. Again, the aqueous layer was acidified using 2N HCl (pH˜3), which gave a white precipitate which was collected by filtration to give 3-(4-bromophenyl)-mercapto-propionic acid (1.08 g, 50%). ¹H NMR (DMSO-d6) δ (ppm): 2.48 (t, 2H), 3.10 (t, 2H), 7.26 (d, 2H), 7.47 (d, 2

The carboxylic acid was converted to the thiochromanone by treatment with polyphosphoric acid.

b) 6-Bromo-thiochroman-4-one from part a) above was converted through to final Compound No. 627 in three steps using similar conditions to those described in Example 36, parts a) to c). Thus preparation of the boronic acid pinacol ester was followed by Suzuki coupling with 6-bromopicolinic acid and then benzothiazole-2-hydrazone formation. NMR details for the final compound are provided in Table 12.

### Example 49

### 6-[9-(Benzothiazol-2-yl-hydrazono)-6,7,8,9-tetrahydro-5H-benzocyclohepten-2-yl]-pyridine-2-carboxylic acid (Compound No 628)

a) 5-(4-Bromophenyl)-pentanoic acid

Aluminium trichloride (1.33 g, 10.0 mmol) was added to bromobenzene (7.46 g, 47.5 mmol). Glutaric acid monoethyl ester chloride (0.89 g, 5 mmol) was added dropwise at 0° C. The reaction mixture was stirred at room temperature during 3.5 hours after which the mixture was poured into an icewater/2N HCl mixture (150 mL). The mixture was extracted with ethyl acetate (2×100 mL). The combined organic layers were washed with water and then brine. Drying (Na₂SO₄) and concentration resulted in a residue which was taken up into 2N NaOH (4 mL) and methanol (7 mL). The mixture was refluxed for 1.5 hours and after concentration, the residue was acidified with 2N HCl (pH˜3) to form a white precipitate which was collected by filtration to afford 5-(4-bromophenyl)-5-oxo-pentanoic acid (990 mg, 73%). ¹H NMR (d₆DMSO) δ (ppm): 1.78 (t, 3H), 2.23 (t, 3H), 3.00 (t, 3H), 7.69(d, 2H), 7.85 (d, 2H). To a mixture of the keto-carboxylic acid (970 mg, 3.58 mmol) in trifluoroacetic acid (2 mL) was added dropwise triethylsilane (1.4 mL, 18.9 mmol). The mixture was stirred overnight at 55° C. After concentration, the mixture was basified (1N NaOH) and extracted with diethyl ether (100 mL). The aqueous layer was then acidified (1N HCl) to give a precipitate which was collected by filtration to give the desired product (769 mg, 84%). ¹H NMR (d₆DMSO) δ (ppm): 1.48 (m, 4H); 2.18 (t, 2H); 2.52 (t, 2H); 7.12 (d, 2H); 7.41 (d, 2H).

b) 3-Bromo-6,7,8,9-tetrahydro-5H-benzocyclohepten-5-one

The carboxylic acid from part b) was converted to the bromo-benzocycloheptenone by treatment with polyphosphoric acid using standard conditions.

c) 3-Bromo-6,7,8,9-tetrahydro-5H-benzocyclohepten-5-one from part b) above was converted through to final Compound No. 628 in three steps using similar conditions to those described in Example 36, parts a) to c). Thus preparation of the boronic acid pinacol ester was followed by Suzuki coupling with 6-bromopicolinic acid and then benzothiazole-2-hydrazone formation. NMR details for the final compound are provided in Table 12.

### Example 50

### 6-[8-(Benzothiazol-2-yl-hydrazono)-5,67,8-tetrahydro-naphthalen-2-yl]-pyridine-2-carboxylic acid 2-morpholin-4-yl-ethyl ester (Compound No. 629)

a) 6-(8-Oxo-5,6,7,8-tetrahydronaphthalen-2-yl)-pyridine-2-carboxylic acid (described in Example 36) (300 mg, 1.12 mmol) was coupled with 4-(2-hydroxyethyl)morpholine (137 μL, 1.12 mmol) using EDC (277 mg) and DMAP (177 mg) in anhydrous dichloromethane (5 mL). The crude product was purified by column chromatography (10/1 dichloromethane-methanol), to yield 277 mg (65%).

b) The product from above (272 mg, 0.716 mmol) was heated under reflux with 2-benzothiazole hydrazine (118 mg, 0.716 mmol) in ethanol (3 mL) for 5 hours. The product (163 mg, 43%) was isolated after purification on the Flash Master (1/1 ethyl acetate/petroleum ether to ethyl acetate only). NMR details for the final compound are provided in Table 12.

### Example 51

### 6-[4-(Benzothiazol-2-yl-hydrazono)-2,3-dihydro-4H-1-oxido-benzothiopyran-6-yl]-pyridine-2-carboxylic acid (Compound No 630)

a) 6-Bromo-pyridine-2-carboxylic acid tert-butyl ester

Tosylchloride (4.5 g, 23.6 mmol) was added to a solution of 6-bromopicolinic acid (2.02 g, 10 mmol) and pyridine (5.43 mL, 67.1 mmol) in 18 mL of tert-butanol at 0° C. The reaction was slowly warmed to room temperature and stirred for 16 hours. The milky mixture was poured onto saturated NaHCO₃ solution using a small amount of Et₂O. A solid precipitated and after Et₂O had evaporated, the solid was collected by filtration and dried in a vacuum oven. A white solid was obtained (1.77 g, 69%). NMR ¹H (CDCl₃, ppm): 7.95 (dd, 1H), 7.65-7.58 (m, 2H), 1.6 (s, 9H). LCMS: M+H=257.8.

b) 6-(4-Oxo-thiochroman-6-yl)-pyridine-2-carboxylic acid tert-butyl ester

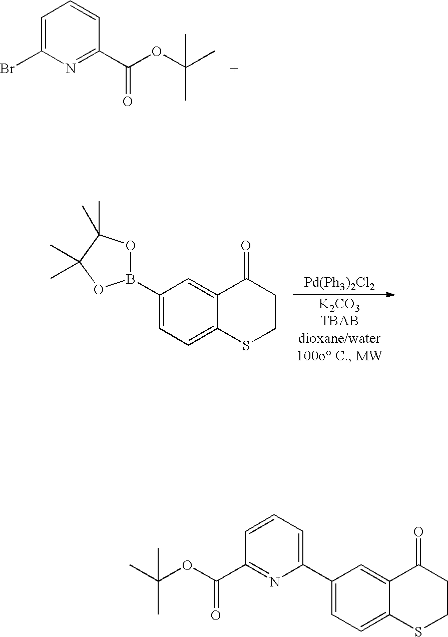

6-Bromo-pyridine-2-carboxylic acid tert-butyl ester (159 mg, 0.78 mmol), 6-(4,4,5,5-tetramethyl-[1,3,2]dioxaborolan-2-yl)-thiochroman-4-one (290 mg, 1 mmol), potassium carbonate (246 mg, 1.6 mmol), tetrabutylammonium bromide (27 mg, 74 μmol) and Pd(PPh₃)₂Cl₂ (36 mg, 52 μmol) were stirred in a mixture of 2.4 mL of dioxane and 1.3 mL of water in a microwave reactor at 100° C. for 1 hour. Water and ethyl acetate were then added. The aqueous layer was extracted three times with ethyl acetate. The combined organic layers were washed with water and brine, dried over Na₂SO₄ and concentrated. The residue was purified by flash chromatography on SiO₂ with Flashmaster purification system. Conditions: Petroleum ether/ethyl acetate 95:5 for 5 minutes, 95:5 to 70:30 in 10 minutes, 70:30 for 5 minutes and 70:30 to 50:50 in 5 minutes. The resulting solid was rinsed with Petroleum ether and Et₂O. A white solid was obtained (205 mg, 69%). NMR ¹H (CDCl₃, ppm): 8.67 (d, 1H), 8.34 (dd, 1H), 7.96-7.92 (m, 2H), 7.84 (t, 1H), 7.39 (d, 1H), 3.29-3.25 (m, 2H), 3.03-2.98 (m, 2H), 1.64 (s, 9H).

c) 6-(2,3-Dihydro-4H-1-benzothiopyran-4-one-1-oxide)-pyridine-2-carboxylic acid tert-butyl ester

A solution of m-CPBA (33 mg of a 77% mixture, 0.15 mmol) in 0.3 mL of chloroform was added by 25 mL aliquots every 5 minutes to a solution of 6-(4-Oxo-thiochroman-6-yl)-pyridine-2-carboxylic acid tert-butyl ester (50 mg, 0.15 mmol) in 0.3 mL of chloroform at room temperature. TLC (Pet. Ether/AcOEt 50:50) indicated complete reaction after 1.5 h. Saturated NaHCO₃ was then added to the reaction and the mixture was stirred vigorously. The organic layer was decanted and the saturated NaHCO₃ treatment was repeated 2 more times followed by one water wash. The chloroform solution was then directly poured onto a column for flash chromatography using SiO₂ and CH₂Cl₂/MeOH 100:0 to 99.5:0.5, then 99:1 to 98.5:1.5. A white solid was obtained (48 mg, 89%). NMR ¹H (d₆-DMSO, ppm): 8.73 (d, J=2.0, 1H), 8.65 (dd, J=8.1 and 2.0 Hz, 1H), 8.03 (dd, J=7.4 and 1.3 Hz, 1H), 7.99 (dd, J=9.6 and 0.4, 1H), 7.98 (dd, J=7.92 and 1.3 Hz, 1H), 7.91 (dd, J=7.9 and 7.44, 1H), 3.57-3.46 (m, 3H), 3.00-2.86 (m, 1H), 1.64 (s, 9H).

d) 6-(2,3-Dihydro-4H-1-benzothiopyran-4-one-1-oxide)-pyridine-2-carboxylic acid

The sulfoxide of the thiopyranyl-pyridine-2-carboxylic acid tert-butyl ester (25 mg, 70 μmol) was treated with 0.5 mL of formic acid and the resulting mixture was stirred at room temperature for 20 hours. It was then concentrated and Et₂O was added. After standing, Et₂O was removed and another treatment with Et₂O was carried out. The resulting solid was then dried in a vacuum oven (17 mg, 81%). NMR ¹H (d₆-DMSO, ppm): 8.75 (d, 1H), 8.60 (dd, 1H), 8.31 (dd, 1H), 8.10 (t, 1H), 8.04 (dd, 1H), 8.01 (d, 1H), 3.74-3.66 (m, 1H), 3.57-3.48 (m, 1H), 3.29-3.19 (m, 1H), 3.01-2.91 (m, 1H). NMR ¹³C (d₆-DMSO, ppm): 192.6, 166.0, 153.8, 148.8, 146.8, 141.0, 139.0, 132.3, 129.8, 129.0, 126.2, 124.3, 123.9, 46.0, 30.4.

e) Hydrazone Formation

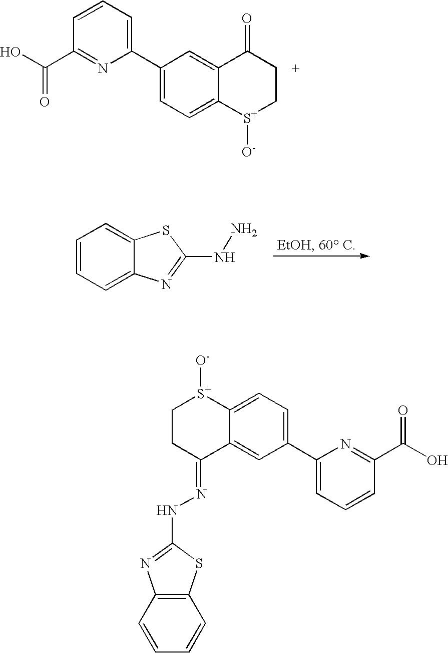

The sulfoxide of the thiopyranyl-pyridine-2-carboxylic (9 mg, 30 μmol) from part d) and benzothiazol-2-yl-hydrazine (5 mg, 0.030 mmol) were stirred in 0.1 mL of EtOH at 60° C. for 16 hours. A white solid formed and after addition of water, it was collected by filtration, rinsed with EtOH and Et₂O and dried in a vacuum oven. Compound No. 630 was obtained as a pale yellow solid (7 mg, 54%). NMR and LCMS details are in Table 12.

### Example 52

### 6-[4-(Benzothiazol-2-yl-hydrazono)-2,3-dihydro-1,1-dioxido-benzothiopyran-6-yl]-pyridine-2-carboxylic acid (Compound No 631)

a) 6-(1,1,4-Trioxo-1-thiochroman-6-yl)-pyridine-2-carboxylic acid t-butyl ester

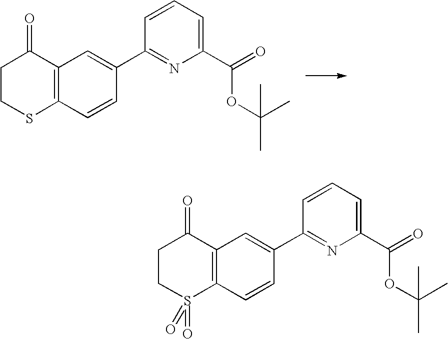

m-Chloroperbenzoic acid (m-CPBA, max. 77%, 112 mg, 0.50 mmol) in dichloromethane (0.5 mL) was added slowly to a solution of the ketone from Example 51, part b) (86 mg, 0.25 mmol) in dichloromethane (0.5 mL) at 0° C. The mixture was allowed to warm to room temperature and stirred for 3 hours. Then, a saturated bicarbonate solution was added and the mixture was vigorously stirred. The layers were separated and the aqueous layer was extracted with dichloromethane (2×5 mL). The combined organic layers were washed with water and brine respectively. Drying (Na₂SO₄) and concentration gave a residue which was purified by flash chromatography (gradient of 50 to 0% petroleum ether in ethylacetate as eluent) to afford the desired sulfone (60 mg, 64%). ¹H NMR (CDCl₃) δ (ppm): 1.62 (s, 9H); 3.42 (t, 2H); 3.71 (t, 2H); 7.88-8.07 (m, 4H); 8.60 (d, 1H); 8.63 (s, 1H).

b) Hydrazone Formation and Ester Hydrolysis

The ketone from part a) was converted to the benzothiazole hydrazone under standard conditions. The resultant tert-butylester of Compound No. 631 (15 mg, 0.029 mmol) was stirred in formic acid (0.2 mL) at room temperature overnight. Following concentration, the residue was triturated with hot methanol and filtered to afford the desired carboxylic acid (11 mg, 82%). NMR and LCMS details for the product are listed in Table 12.

### Example 53

The ester Compound No. 632 was prepared following a similar sequence of reactions to those described for Compound No. 629 in Example 50 above. LCMS details for the product are listed in Table 12.

### Example 54

### 6-[4-(Benzothiazol-2-yl-hydrazono)-2-trifluoromethyl-1,2,3,4-tetrahydro-quinolin-6-yl]-pyridine-2-carboxylic acid (Compound No. 633)

a) 2-Trifluoromethyl-4-oxo-1,2,3,4-tetrahydro-quinoline-6-boronic acid pinacol ester

6-Bromo-2-trifluoromethyl-2,3-dihydro-1H-quinolin-4-one (20 mg, 68 μmol, prepared according to Gong and Kato, 2004), bis(pinacolato)diboron (19 mg, 75 μmol), potassium acetate (20 mg, 0.2 mmol) and Pd(dppf)₂Cl₂.CH₂Cl₂ (2 mg, 2 mol %) were heated in toluene at 150° C. in a microwave reactor for 8 minutes. The reaction mixture was then diluted with ethyl acetate and water. The aqueous layer was extracted three times with ethyl acetate. The organic phases were washed with water and brine, dried over Na₂SO₄ and concentrated. The residue was purified by flash chromatography on SiO₂ using petroleum ether and ethyl acetate 90:10 to 70:30. A yellow oil was obtained (7 mg, 30%). NMR ¹H (CDCl₃, ppm): 8.3 (s, 1H), 7.75 (dd, 1H), 6.79 (d, 1H), 4.67 (br s, 1H), 4.19-4.09 (m, 1H), 2.99-2.82 (m, 2H), 1.30 (s, 12H).

b) 6-(4-Oxo-2-trifluoromethyl-1,2,3,4-tetrahydro-quinolin-6-yl)-pyridine-2-carboxylic acid

The boronic acid ester from part a) (7 mg, 21 μmol), 6-bromopicolinic acid (4 mg, 21 μmol), Pd(PPh₃)₂Cl₂ (1 mg, 5 mol %) were stirred in a mixture of 9 μL of 2M sodium carbonate and 0.2 mL of DME/EtOH/H₂O 1:1:1 at 90° C. for 1 hour. The reaction mixture was then concentrated and water and ethyl acetate were added. The aqueous phase was extracted twice with ethyl acetate. Dilute HCl was added to the aqueous phase, which became cloudy. Ethyl acetate was added and the acidic aqueous phase was extracted twice with ethyl acetate. The organic phase was concentrated and the resulting solid was dried to give the product (6 mg, 85%). NMR ¹H (CD₃OD, ppm): 8.36 (s, 1H), 8.11-8.08 (m, 2H), 7.87-7.77 (m, 2H), 7.01 (d, 1H), 4.43-4.36 (m, 1H), 3.20-3.12 (m, 1H), 2.89-2.82 (m, 1H).

c) Hydrazone Formation

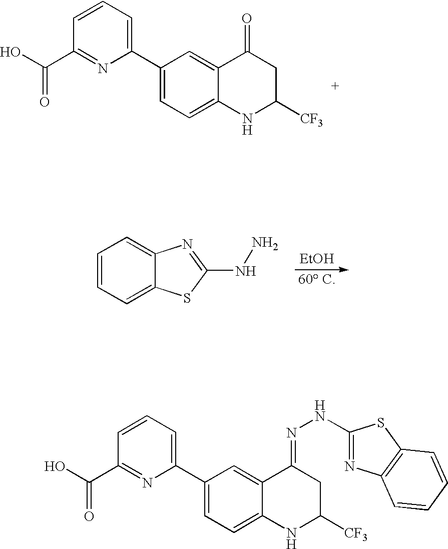

6-(4-Oxo-2-trifluoromethyl-1,2,3,4-tetrahydro-quinolin-6-yl)-pyridine-2-carboxylic acid (6 mg, 20 μmol) and benzothiazol-2-yl-hydrazine (3.4 mg, 20 μmol) were stirred in EtOH at 60° C. for 16 hours. A yellow solid formed which was collected by filtration and dried. NMR details are in the Table 12.

### Example 55

### 6-[4-(Benzothiazol-2-yl-hydrazono)-2,2-dimethyl-chroman-4-on-6-yl]pyridine-2-carboxylic acid (Compound No 634)

a) 2,2-Dimethyl-4-oxo-chromanyl-6-boronic acid pinacol ester

6-Bromo-2,2-dimethyl-chroman-4-one (250 mg, 0.95 mmol, prepared according to Sebille et al., 2005), bis(pinacolato)diboron (275 mg, 1.05 mmol), potassium acetate (290 mg, 3 mmol) and Pd(dppf)₂Cl₂.CH₂Cl₂ (90 mg, 2 mol %) were heated in toluene at 150° C. in a microwave reactor for 8 minutes. The reaction mixture was then diluted with ethyl acetate and water. The aqueous layer was extracted three times with ethyl acetate. The organic phases were washed with water and brine, dried over Na₂SO₄ and concentrated. The residue was purified by flash chromatography on SiO₂ with flashmaster purification system using petroleum ether and ethyl acetate (95:5 for 5 minutes, 95:5 to 85:15 in 7 minutes, 85:15 to 70:30 in 15 minutes). A yellow solid was obtained (293 mg, quant.). NMR ¹H (CDCl₃, ppm): 8.32 (d, J=1.4 Hz, 1H), 7.84 (dd, 1H), 6.87 (dd, 1H), 2.69 (s, 2H), 1.42 (s, 6H), 1.29 (s, 12H).

b) 6-(2,2-Dimethyl-4-oxo-chroman-6-yl)-pyridine-2-carboxylic acid

The boronic acid ester from part a) (293 mg, 0.97 mmol), 6-bromopicolinic acid (151 mg, 0.8 mmol), Pd(PPh₃)₂Cl₂ (23 mg, 5 mol %) were stirred in a mixture of 371 μL of 2M sodium carbonate and 1.5 mL of DME/EtOH/H₂O 1:1:1 at 90° C. for 1 hour. The reaction was then concentrated, water and ethyl acetate were added and the aqueous phase was extracted twice with ethyl acetate. HCl (2M) was added to the aqueous phase leading to a precipitate. The solid was collected by filtration, rinsed with water and dried in a vacuum oven (131 mg, 60%). NMR ¹H (d₆-DMSO, ppm): 8.47 (s, 1H), 8.34 (dd, J=8.7 and 2.4 Hz, 1H), 8.14 (dd, J=7.8 and 0.9 Hz, 1H), 8.00 (t, J=7.5 Hz, 1H), 7.92 (dd, J=6.6 and 0.9 Hz, 1H), 7.10 (d, J=8.7 Hz, 1H), 2.84 (s, 2H), 1.40 (s, 6H).

c) Hydrazone Formation

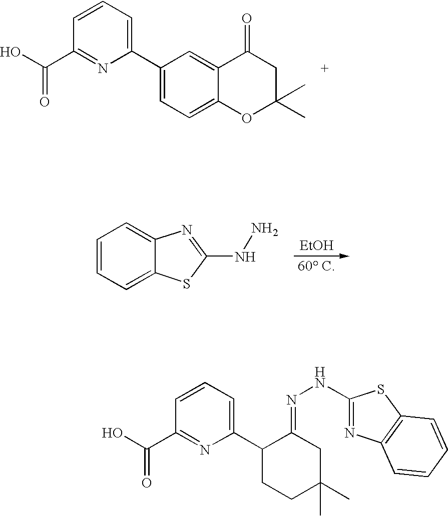

6-(2,2-Dimethyl-4-oxo-chroman-6-yl)-pyridine-2-carboxylic acid (70 mg, 0.24 mmol) and benzothiazol-2-yl-hydrazine (39 mg, 0.24 mmol) were stirred in EtOH at 60° C. for 16 hours. A white solid formed which was filtered, rinsed with EtOH and Et₂O and dried in a vacuum oven (75 mg, 70%). LCMS and NMR details are listed in Table 12.

### Example 56

### 6[-8-(Benzothiazol-2-yl-hydrazono)-7-methyl-5,6,7,8-tetrahydro-naphthalen-2-yl]-pyridine-2-carboxylic acid (Compound No 635)

a) 7-Bromo-2-methyl-3,4-dihydro-2H-naphthalen-1-one

To a solution of LDA (2M, 2.2 mL) in anhydrous THF (16 mL) at −78° C. 7-bromo-3,4-dihydro-1(2H)-naphthalenone (1.0 g, 4.44 mmol) in THF (5 mL) was added dropwise and the mixture was stirred at −78° C. for 30 minutes. Iodomethane (276 μL, 4.44 mL) was added and the reaction mixture left stirring for 18 hours at ambient temperature. Saturated ammonium chloride was added and then diethyl ether. The organic layer was washed with brine followed by drying and evaporation of solvent. The crude product was purified by column chromatography (6/1 petroleum ether-ethyl acetate) to obtain the mono- (424 mg, 40%) and di-methylated (112 mg, 10%) products.

b) 7-Bromo-2-methyl-3,4-dihydro-2H-naphthalen-1-one from part a) was converted through to final Compound No. 635 in three steps using similar conditions to those described in Example 36, parts a) to c). Thus preparation of the boronic acid pinacol ester was followed by Suzuki coupling with 6-bromopicolinic acid and then benzothiazole-2-hydrazone formation. NMR details for the final compound are provided in Table 12.

### Example 57

### 6-[8-(Benzothiazol-2-yl-hydrazono)-7,7-dimethyl-5,6,7,8-tetrahydro-naphthalen-2-yl]-pyridine-2-carboxylic acid (Compound No 636)

a) 7-Bromo-2,2-dimethyl-3,4-dihydro-2H-naphthalen-1-one was obtained as the minor product from Example 56, part a).

b) 7-Bromo-2,2-dimethyl-3,4-dihydro-2H-naphthalen-1-one from part a) was converted through to final Compound No. 636 in three steps using similar conditions to those described in Example 36, parts a) to c). Thus preparation of the boronic acid pinacol ester was followed by Suzuki coupling with 6-bromopicolinic acid and then benzothiazole-2-hydrazone formation. NMR details for the final compound are provided in Table 12.

### Example 58

### 6-[8-(Benzothiazol-2-yl-hydrazono)-6-methyl-5,6,7,8-tetrahydro-naphthalen-2-yl]-pyridine-2-carboxylic acid (Compound No 637)

a) 4-(4-Bromophenyl)-3-methyl-butyraldehyde

4-bromo-1-iodobenzene (2.5 g, 8.84 mmol), lithium acetate (1.46 g, 22.1 mmol), lithium chloride (375 mg, 8.8 mmol), 3-methyl-3-buten-1-ol (761 mg, 8.8 mmol), tetrabutylammonium chloride hydrate (4.91 g, 17.7 mmol) and palladium(II)acetate (119 mg, 4.9 mmol) were stirred for 4 days in dimethyl formamide (17 mL) at 70° C. The reaction mixture was cooled and quenched with saturated ammonium chloride solution and extracted with diethylether (2×200 mL). The combined organic layers were washed with brine, dried (Na₂SO₄) and concentrated. The residue was purified by flash chromatography (gradient of 0 to 10% ethylacetate in petroleum ether as eluent) to afford the carboxyaldehyde (975 mg, 46%). ¹H NMR (d₆DMSO) δ (ppm): 0.93 (d, 3H); 2.20-2.60 (m, 5H); 7.01 (d, 2H); 7.39 (d, 2H); 9.70 (s, 1H).

b) 4-(4-Bromophenyl)-3-methyl-butyric acid

Periodic acid (1.0 g, 4.4 mmol) was stirred in acetonitrile (35 mL) for 15 minutes. The aldehyde from part a) (0.97 g, 4.0 mmol) dissolved in acetonitrile (4 mL) was added at 0° C. followed by pyridinium chlorochromate (18 mg, 0.08 mmol) in acetonitrile (4 mL). The reaction mixture was stirred at room temperature for 1.5 hours. Next, ethylacetate (250 mL) was added and the mixture was subsequently washed with a 1:1 brine:water solution, saturated sodium hydrogensulphate and brine. Drying (Na₂SO₄) and concentration afforded the desired carboxylic acid (990 mg, 96%). ¹H NMR (d₆DMSO) δ (ppm): 0.94 (d, 3H); 2.12-2.38 (m, 3H); 2.45 (dd, 1H); 2.59 (dd, 1H); 7.02 (d, 2H); 7.39 (d, 2H).

c) 7-Bromo-3-methyl-3,4-dihydro-2H-naphthalen-1-one was prepared by treatment of the butyric acid from part b) with polyphosphoric acid under standard conditions.

d) 7-Bromo-3-methyl-3,4-dihydro-2H-naphthalen-1-one from part c) above was converted into final Compound No. 637 in three steps using similar conditions to those described in Example 36, parts a) to c). Thus preparation of the boronic acid pinacol ester was followed by Suzuki coupling with 6-bromopicolinic acid and then benzothiazole-2-hydrazone formation. NMR details for the final compound are provided in Table 12.

### Example 59

### 6-[5-(Benzothiazol-2-yl-hydrazono)-2,3,4,5-tetrahydro-benzo[b]oxepin-7-yl]-pyridine-2-carboxylic acid (Compound No 638)

7-Bromo-3,4-dihydro-2H-benzo[b]oxepin-5-one was converted into final Compound No. 638 in three steps using similar conditions to those described in Example 36, parts a) to c). Thus preparation of the boronic acid pinacol ester was followed by Suzuki coupling with 6-bromopicolinic acid and then benzothiazole-2-hydrazone formation. NMR details for the final compound are provided in Table 12.

### Example 60

### 6-[3-(Benzothiazol-2-yl-hydrazono)-1-methyl-2-oxo-2,3-dihydro-1H-indol-5-yl]-pyridine-2-carboxylic acid (Compound No. 639)

5-Bromo-1-methyl-1H-indole-2,3-dione was converted into final Compound No. 639 in three steps using similar conditions to those described in Example 36, parts a) to c). Thus preparation of the boronic acid pinacol ester was followed by Suzuki coupling with 6-bromopicolinic acid and then benzothiazole-2-hydrazone formation. NMR details for the final compound are provided in Table 12.

### Example 61

### 2-[8-(2-Benzothiazol-2-yl-hydrazono)-5,6,7,8-tetrahydronaphthalen-2-yl]-thiazole-4-carboxylic acid (Compound No. 800)

a) 2-(8-Oxo-5,6,7,8-tetrahydro-naphthalen-2-yl)-thiazole-4-carboxylic acid ethyl ester

1-Tetralone-7-boronic acid pinacol ester (250 mg, 0.92 mmol), 2-chlorothiazole-4-carboxylic acid ethyl ester (153 mg, 0.80 mmol), tetrabutylammoniumborohydride (25 mg, 0.085 mmol), potassium carbonate (295 mg, 2.14 mmol) and trans-dichlorobis(triphenylphosphine)palladium(II) (27 mg, 0.038 mmol) were dissolved in dioxane (4 mL) and water (2 mL). Under microwave conditions (120 W) the solution was heated at 100° C. for 2 hours. After cooling the reaction mixture was poured into a water/ethyl acetate mixture (100 mL). After extraction, the aqueous layer was acidified with 2N hydrochloric acid (pH˜3) to give a white precipitate which was collected by filtration to give the desired product (62mg, 22%). ¹H NMR (CDCl₃) 8 (ppm):1.41 (t, 3H), 2.16 (m, 2H), 2.69 (t, 2H), 3.00 (t, 2H), 4.43 (q, 2H), 7.35 (d, 1H), 8.15 (s, 1H), 8.25 (dd, 1H), 8.49 (s, 1H).

b) 2-(8-Oxo-5,6,7,8-tetrahydro-naphthalen-2-yl)-thiazole-4-carboxylic acid

The ester from part a) (56 mg, 0.19 mmol) was stirred in 1N NaOH (2 mL) and MeOH (2 mL) at 90° C. for 2.5 hours. After concentration, 10% citric acid (10 mL) was added and the mixture was extracted with ethyl acetate (2×10 mL). The combined organic layers were washed with brine, dried (Na₂SO₄) and concentrated to afford the desired carboxylic acid (46 mg, 89%). LCMS 274 (M+1).

c) Hydrazone Formation

The keto-acid from part b) was condensed with 2-benzothiazole hydrazine using similar conditions to those described in Example 36 part c). NMR and LCMS details for the product are in Table 12.

### Example 62

### 2-[8-(Benzothiazol-2-yl-hydrazono)-5,6,7,8-tetrahydro-naphthalen-2-yl]-oxazole-4-carboxylic acid (Compound No. 801)

a) 2-(8-Oxo-5,6,7,8-tetrahydro-naphthalen-2-yl)-oxazole-4-carboxylic acid ethyl ester

2-Chloro-oxazole-4-carboxylic acid ethyl ester (50 mg, 0.28 mmol), prepared according to Hodgetts and Kershaw, 2002, 7-(4,4,5,5-Tetramethyl-[1,3,2]dioxaborolan-2-yl)-3,4-dihydro-2H-naphthalen-1-one (94 mg, 0.36 mmol), tetrabutylammonium bromide (10 mg, 0.04 mmol) and Pd(PPh₃)₂Cl₂ (13 mg, 0.02 mmol) in dioxane:water (1.2 mL/0.6 mL) were heated at 100° C. in a microwave reactor for 1 hour. After this time, water and ethyl acetate were added and the aqueous phase was extracted three times with ethyl acetate. The combined organic phases were washed with water and brine and dried over Na₂SO₄. Concentration afforded a brown solid which was triturated with Et₂0. The product was obtained as an off-white solid (43mg, 54%). NMR ¹H (CDCl₃, ppm): 8.72 (s, 1H), 8.31 (s, 1H), 8.28 (dd, 1H), 7.41 (d, 1H), 4.45 (q, 2H), 3.07-3.03 (m, 2H), 2.75-2.71 (m, 2H), 2.25-2.16 (m, 2H), 1.44 (t, 1H).

b) 2-(8-Oxo-5,6,7,8-tetrahydro-naphthalen-2-yl)-oxazole-4-carboxylic acid

Sodium hydroxide (0.2 mL of a 1M aqueous solution) was added to the ethyl ester from part a) (43 mg, 0.15 mmol) dissolved in 0.2 mL of MeOH. The reaction was then heated to 90° C. for a few minutes and all solids disappeared. It was then stirred to room temperature for 1 hour. The reaction was concentrated, water was added, the aqueous layer was washed twice with Et₂O and acidified to pH=1 with HCl (2M). The white solid formed was collected by filtration, rinsed with water and dried in a vacuum oven (23 mg, 61%). NMR ¹H (d₆-DMSO, ppm): 8.80 (s, 1H), 8.4 (s, 1H), 8.10 (dd, 1H), 7.53 (d, 1H), 3.01-2.97 (m, 2H), 2.65-2.61 (m, 2H), 2.09-2.01 (m, 2H).

c) Hydrazone Formation

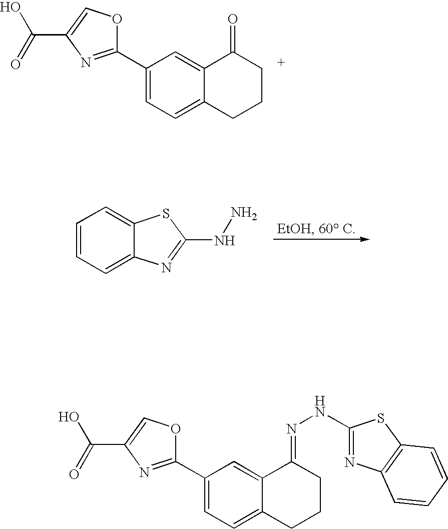

2-(8-Oxo-5,6,7,8-tetrahydro-naphthalen-2-yl)-oxazole-4-carboxylic acid (23mg, 89 μmol) and benzothiazol-2-yl-hydrazine (15 mg, 89 μmol) were stirred in EtOH at 60° C. for 16 hours A white solid formed and after addition of water, it was collected by filtration, rinsed with EtOH and Et₂O and dried to give the product (22 mg, 61%). NMR details are listed in Table 12.

### Example 63

Compound No.'s 802, 803 and 804 were prepared using similar conditions to those described in Examples 61 and 62. Thus the required boronic acid pinacol ester was coupled with the appropriate chloro-heterocycle followed by ester hydrolysis in the case of the thiazole derivatives and then benzothiazole-2-hydrazone formation. NMR details for the final compounds are provided in Table 12.

### Example 64

Compound No.'s 900, 901 and 902 were prepared from 2-hydrazino-benzoselenazole (G. A. Reynolds et al., 1959) using similar conditions to those described for the corresponding benzothiazole hydrazones. NMR details for the final compounds are provided in Table 12.

### Example 65

### 6-[8-(Benzothiazol-2-yl-hydrazono)-5,6,7,8-tetrahydro-naphthalen-2-yl]-3-hydroxy-pyridine-2-carboxylic acid (Compound No. 1000)

a) Methyl 6-bromo-3-hydroxypicolinic acid ester was prepared using similar bromination conditions to the published method (PCT patent WO 2005/009962).

b) The bromo-picolinate from part a) above was converted through to final Compound No. 1000 in three steps using similar reactions to those described in Example 61, parts a) to c). Thus Suzuki coupling of the tetralone-boronic acid pinacol ester with 6-bromo-3-hydroxypicolinic acid methyl ester was followed by ester hydrolysis and then benzothiazole-2-hydrazone formation. LCMS details for the final compound are provided in Table 12.

### Example 66

### 6-[8-(Benzothiazol-2-yl-hydrazono)-5,6,7,8-tetrahydro-naphthalen-2-yl]-3-acetoxy-pyridine-2-carboxylic acid (Compound No. 1001)

a) Acetylation of 3-hydroxy-6-(8-oxo-5,6,7,8-tetrahydro-naphthalen-2-y1)-pyridine-2-carboxylic acid

A mixture of the 3-hydroxypicolinate (20 mg, 0.066 mmol), pyridine (20 ul, 0.20 mmol), acetic anhydride (1 ml) and dichloroethane (1 mL) were heated at 50° C. for 4 hours. The solvent was removed in vacuo and water was added. The aqueous mixture was extracted with ethyl acetate (2×3 ml), the organic layer was dried (MgSO₄) and concentrated to give the crude 3-O-acetyl-picolinic acid product.

b) Hydrazone Formation

A mixture of the picolinate from part a) (20 mg, 0.059 mmol) and benzothiazole-2-hydrazine (9.6 mg, 0.065 mmol) in 1 mL of acetic acid was heated at 60° C. for 16 hours. Water was added and the solid precipitate was collected by filtration, washed with water and dried to give the title compound as a pale brown solid (15 mg, 53%). LCMS details for Compound No. 1001 are provided in Table 12.

### Example 67

### 6-18-(Benzothiazol-2-yl-hydrazono)-5,6,7,8-tetrahydro-naphthalen-2-yl]-3-(3-phenoxypropoxy)-pyridine-2-carboxylic acid (Compound No. 1002)

a) 6-Bromo-3-(3-phenoxy-propoxy)-pyridine-2-carboxylic acid methyl ester

A mixture of 6-bromo-3-hydroxypicolinic acid methyl ester (100 mg, 0.43 mmol), sodium hydride (60% dispersion in mineral oil) (17.3 mg, 0.43 mmol), 3-phenoxypropyl bromide (68 μL, 0.43 mmol), in dimethyl acetamide (1.5 mL) was heated at 90° C. for 3 hours. The reaction was allowed to cool, poured onto water and then extracted with ethyl acetate (2×3 mL). The organic layer was dried (MgSO₄) and concentrated in vacuo. The resulting residue was purified by silica chromatography eluting with 100% petroleum ether to 30% ethyl acetate/petroleum ether to obtain a white solid (95 mg, 60%). ¹H NMR (300 MHz, CDCl₃) δ: 7.48 (1H, d), 7.24-7.21 (3H, m), 6.91-6.86 (3H, m), 4.19 (2H, t), 4.15 (1H, t), 3.88 (3H, s), 2.30-2.22 (2H, m).

b) Suzuki Coupling

A mixture of the methyl picolinate from part a) (90 mg, 0.025 mmol), 8-Oxo-5,6,7,8-tetrahydro-naphthalene-2-boronic acid pinacol ester (129 mg, 0.028 mmol), PdCl₂(PPh₃)₂ (4.3 mg, 2.5 mol %), tetrabutylammonium bromide (9 mg, 0.0025 mmol), potassium carbonate (118 mg, 0.088 mmol) in a dioxane:H₂O (2:1 mL) mixture was heated at 100° C. under microwave irradiation for 90 minutes. The reaction was cooled down, diluted with ethyl acetate and poured onto water. The aqueous phase was extracted with ethyl acetate and then acidified with 2N hydrochloric acid solution. The solid that precipitated was filtered off at the pump and washed with water to afford an off-white solid (30 mg, 29%) (the organic layer contained the product ester). LCMS: M+H 418; >95% purity.

c) Hydrazone Formation

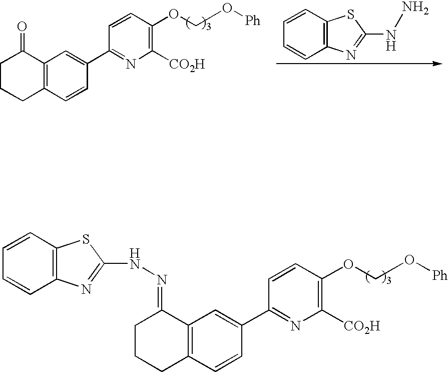

A mixture of the picolinate (30 mg, 0.072 mmol) and benzothiazole-2-hydrazine (15 mg, 0.079 mmol) in 1 mL of acetic acid was heated at 60° C. for 16 hours. After this time water was added and a solid precipitated. It was collected by filtration, washed with water and dried in a vacuum oven to give the title compound as a pale brown solid (28 mg, 73%). LCMS details for Compound No. 1002 are provided in Table 12.

### Example 68

### 6-[8-(Benzothiazol-2-yl-hydrazono)-5,6,7,8-tetrahydro-naphthalen-2-yl]-3-methoxy-pyridine-2-carboxylic acid (Compound No. 1003)

a) 3-Hydroxy-6-(8-Oxo-5,6,7,8-tetrahydro-naphthalen-2-yl)-pyridine-2-carboxylic acid methyl ester

A mixture of methyl 6-bromo-3-hydroxypicolinate (100 mg, 0.43 mmol), 7-pinacloatoborane-tetralone (129 mg, 0.47 mmol), PdCl₂(PPh₃)₂ (7.5 mg, 2.5 mol %), tetrabutylammonium bromide (14 mg, 0.043 mmol), caesium fluoride (145 mg, 0.95 mmol) in dioxane:H₂O (2:1, mL) was heated at 100° C. under microwave irradiation for 90 minutes. The reaction was cooled, diluted with ethyl acetate and poured onto water. The aqueous phase was extracted with ethyl acetate (2×3 mL), the organic layer was dried (MgSO₄) and concentrated in vacuo. The residue was triturated with diethyl ether and the solid was filtered to afford an off-white solid (40 mg, 31%). ¹H NMR (d₆-DMSO) δ: 10.55 (1H, bs), 8.42 (1H, d), 8.13 (1H, dd), 8.07 (1H, dd), 7.48 (1H, d), 7.43 (1H), 3.90 (3H, s), 2.96 (2H, t), 2.61 (2H, t), 2.07-2.01 (2H, m).

b) 3-Methoxy-6-(8-Oxo-5,6,7,8-tetrahydro-naphthalen-2-yl)-pyridine-2-carboxylic acid methyl ester

A mixture of the methyl picolinate from part a) (30 mg, 0.10 mmol), potassium carbonate (28 mg, 0.20 mmol), iodomethane (19 μL, 0.30 mmol), in acetone (2 mL) was heated at reflux for 16 hours. The reaction was cooled, diluted with ethyl acetate and poured onto water. The aqueous phase was extracted with ethyl acetate (2×3 mL) and the organic layer was dried (MgSO₄) and concentrated in vacuo to give the product as a white solid. LCMS: M+H 312; >95% purity

c) Hydrazone Formation and Ester Hydrolysis

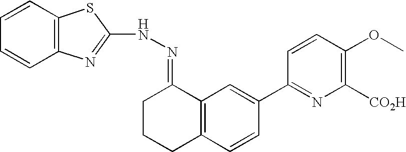

A mixture of the picolinate from part b) (20 mg, 0.064 mmol) and benzothiazole-2-hydrazine (11 mg, 0.070 mmol) in 1 mL of acetic acid was heated at 60° C. for 16 hours. Water was added and a solid precipitated which was collected by filtration, washed with water and dried in a vacuum oven to give the product as a pale brown solid. The solid was dissolved in tetrahydrofuran (1 mL) and sodium hydroxide solution (2N, 1 mL) was added and the solution was stirred for 16 hours. Ethyl acetate was added to the solution and the layers separated. The aqueous layer was acidified and the solid that precipitated was filtered to give Compound No. 1003 as a pale brown solid (16 mg, 56%). LCMS details for Compound No. 1003 are provided in Table 12.

### Example 69

### 6-[4-(Benzothiazol-2-yl-hydrazono)-4,5,6,7-tetrahydro-benzo[b]thiophen-2-yl]-pyridine-2-carboxylic acid (Compound No. 1100)

a) 2-Bromo-6,7-dihydro-5H-benzo[b]thiophen-4-one

Bromine (44 uL, 0.85 mmol) was added to a solution of the thiophene (100 mg, 0.66 mmol) in acetic acid (0.5 mL) and water (0.5 mL) at 0° C. After 2 hours stirring at 0° C., ethyl acetate (20 mL and water (20 mL) were added. The organic layer was washed with brine, dried (Na₂SO₄) and concentrated. The residue was purified by flash chromatography with a 6:1 mixture of petroleum ether and ethyl acetate as eluent to afford the desired bromothiophene (87 mg, 57%).

b) The bromothiophene from part a) above was converted through to final Compound No. 1100 in three steps using similar conditions to those described in Example 36, parts a) to c). Thus preparation of the boronic acid pinacol ester was followed by Suzuki coupling with 6-bromopicolinic acid and then benzothiazole-2-hydrazone formation. LCMS details for the final compound are provided in Table 12.

### Example 70

### 3-[4-(Benzothiazol-2-yl-hydrazono)-4,5,6,7-tetrahydro-indazol-2-yl]-benzoic acid (Compound No. 1101)

a) 2-Methylthio-7,8-dihydro-6H-quinazolin-5-one was prepared following the literature method (A. Bajnati, et al., Bull. Soc. Chim. Fr., 1987, p 318).

b) 3-(4-Oxo-4,5,6,7-tetrahydro-indazol-2-yl)-benzoic acid was prepared by reaction of the quinazoline from part a) with 3-hydrazino-benzoic acid following the reaction conditions described in the literature for similar conversions (A. Bajnati, et al., 1987). ¹H NMR (d₆-DMSO) δ: 2.0-2.1 (m, 2H), 2.45 (m, 2H), 2.84 (m, 2H), 7.60 (t, 1H), 7.86-7.89 (m, 1H), 8.10-8.14 (m, 1H), 8.39 (bs), 9.05 (s, 1H)

c) Hydrazone formation was carried out using the normal conditions to give Compound No. 1101 as a pale brown solid. NMR and LCMS indicate a 1:1 mixture of cis/trans isomers. Details are provided in Table 12.

### Example 71

The compounds of the invention which are listed in Tables 1 to 11 above were generally isolated as pure solids by filtration following the hydrazone formation reaction. The compounds were characterized by their ¹H nmr spectra and in some cases also by their mass spectra. For convenience the nmr data are recorded in Table 12 below.

TABLE 12

| Compound | NMR data: Proton Chemical Shift δ in ppm (recorded in d₆DMSO unless |
|---|---|
| No. | otherwise noted) or Mass spectral data |
| 1 | 2.28 (s, 3H), 2.59 (s, 3H), 6.86 (d, 1H), 6.98 (d, 1H), 7.05 (m, 1H), |
| | 7.2-7.3 (m, 1H), 7.37 (dd, 1H), 7.66 (dd, 2H), 7.77 (d, 1H). |
| 2 | 2.29 (s, 3H), 6.98 (d, 1H), 7.05 (dd, 1H), 7.2-7.3 (m, 2H), 7.58 (dd, 1H), |
| | 7.67 (d, 1H), 7.86 (d, 1H), 7.98 (d, 1H), 8.29 (s, 1H) |
| 3 | 2.29 (s, 3H), 7.01 (d, 1H), 7.06 (dd, 1H), 7.2-7.3 (m, 2H), 7.31 (d, 1H), |
| | 7.6-7.7 (m, 2H), 7.83 (dd, 1H), 8.51 (d, 1H). |
| 4 | 2.28 (s, 3H), 6.99 (d, 1H), 7.05 (m, 1H), 7.22 (d, 1H), 7.2-7.3 (m, 2H), |
| | 7.6-7.7 (m, 2H), 7.85 (dd, 1H), 8.11 (d, 1H). |
| 5 | 2.30 (s, 3H), 7.0-7.2 (m, 2H), 7.2-7.3 (m, 2H), 7.48 (d, 1H), 7.64 (d, 1H), |
| | 8.02 (dd, 1H), 8.07-8.13 (m, 2H) |
| 6 | 1.12 (t, 3H), 2.80 (q, 2H), 6.98 (d, 1H), 7.05 (m, 1H), 7.18 (d, 1H), |
| | 7.26 (m, 2H), 7.58 (dd, 1H), 7.67 (d, 1H), 7.86 (d, 1H), 7.97 (d, 1H), 8.29 (s, 1H). |
| 7 | 2.34 (s, 3H), 7.03-7.07 (m, 1H), 7.22-7.29 (m, 2H), 7.47 (d, 1H), 7.67 (d, |
| | 1H), 7.80 (d, 1H), 7.98-8.04 (m, 2H), 8.01 (s, 1H). |
| 8 | 2.33 (s, 3H), 7.02-7.05 (m, 1H), 7.21-7.23 (m, 2H), 7.40 (d, 1H), 7.55 (d, |
| | 1H), 7.58 (dd, 1H), 7.67 (d, 1H), 7.83 (d, 1H), 7.99 (s, 1H). |
| 9 | 0.91 (d, 6H), 2.08 (m, 1H), 2.71 (d, 2H), 6.94 (d, 1H), 7.03 (m, 1H), |
| | 7.2-7.3 (m, 3H), 7.5-7.6 (m, 2H), 7.85 (d, 1H), 8.12 (s, 1H) |
| 10 | 1.23 & 1.28 (2xd, total 6H), 3.80 (m, 4H), 6.91-7.32 (m, 4H), |
| | 7.55-7.67 (m, 3H), 7.81-7.89 (m, 1H), 8.09 (2s, 1H) |
| 11 | 1.11 (t, 3H), 2.77 (q, 2H), 6.99 (d, 1H), 7.05 (m, 1H), 7.21-7.25 (m, 3H), |
| | 7.60-7.65 (m, 2H), 7.85 (dd, 1H), 8.11 (d, 1H) |
| 12 | 2.30 (s, 3H), 3.87 (s, 3H), 6.30 (d, 1H), 6.63 (d, 1H), 6.98-7.04 (m, 1H), |
| | 7.19-7.26 (m, 2H), 7.55-7.59 (m, 2H), 7.63 (d, 1H), 7.78 (d, 1H) |
| 13 | 2.27 (s, 3H), 6.59 (dd, 1H), 6.67 (dd, 1H), 7.01 (dd, 1H), 7.21 (dd, 1H), |
| | 7.24 (dd, 1H), 7.49 (d, 1H), 7.66 (d, 1H), 7.88 (d, 1H), 8.16 (s, 1H), |
| | 11.20 (s, 1H). |
| 14 | 0.89 (t, 3H), 1.30-1.41 (m, 2H), 1.47-1.57 (m, 2H), 2.78 (t, 2H), 6.95 (d, |
| | 1H), 7.00-7.06 (m, 1H), 7.20-7.29 (m, 3H), 7.60-7.67 (m, 2H), 7.84 (dd, |
| | 1H), 8.11 (d, 1H) |
| 15 | 2.30 (t, 3H), 6.99 (d, 1H), 7.02-7.08 (m, 1H), 7.2-7.3 (m, 2H), 7.33 (d, 1H), |
| | 7.65 (d, 1H), 8.35 (s, 1H), 8.47 (s, 2H); MS 422 (M + 1) |
| 16 | 2.26 (s, 3H), 6.71 (d, 1H), 6.82-6.87 (m, 1H), 6.86 (d, 1H), 7.03 (dd, 1H), |
| | 7.23 (dd, 1H), 7.27-7.29 (m, 1H), 7.60-7.67 (m, 2H), 8.11 (s, 1H). |
| 17 | 2.29 (s, 3H), 6.98 (d, 1H), 7.11 (d, 1H), 7.20-7.27 (m, 2H), 7.46-7.66 (br. |
| | m, 3H), 7.59 (d, 2H), 7.95 (d, 2H), 8.09 (dd, 1H), 8.43 (s, 1H), 8.79 (d, |
| | 1H). |
| 18 | 2.09 (s, 3H), 2.27 (s, 3H), 6.95 (d, 1H), 7.04 (dd, 1H), 7.15-7.25 (m, 2H), |
| | 7.70 (d, 1H), 7.80 (dd, 1H), 8.35 (d, 1H), 8.53 (d, 1H) |
| 19 | 2.35 (s, 3H), 3.62 (s, 2H), 3.70 (s, 3H), 6.87 (d, 2H), 6.95 (d, 2H), 7.03 (dd, |
| | 2H), 7.25 (d, 3H), 7.72 (d, 1H), 7.82 (d, 1H), 8.34 (s, 1H), 8.55 (d, 1H). |
| 20 | 2.28 (s, 3H), 6.98 (d, 1H), 7.0-7.1 (m, 1H), 7.22 (d, 1H), 7.3-7.4 (m, 1H), |
| | 7.62 (d, 1H), 7.66 (dd, 1H), 7.86 (dd, 1H), 8.12 (d, 1H) |
| 21 | 2.28 (s, 3H), 2.57 (s, 3H), 6.86 (d, 1H), 6.99 (d, 1H), 7.05-7.15 (m, 1H), |
| | 7.3-7.4 (m, 2H), 7.6-7.7 (m, 2H), 7.76 (dd, 1H) |
| 22 | 2.30 (s, 3H), 7.00 (d, 1H), 7.07 (dd, 1H), 7.24 (dd, 1H), 7.27 (d, 2H), |
| | 7.56 (dd, 1H), 7.61 (d, 1H), 7.69 (d, 1H), 7.99 (d, 1H). |
| 23 | 2.29 (s, 3H), 7.00 (d, 1H), 7.03-7.08 (m, 1H), 7.2-7.3 (m, 2H), 7.31 (d, |
| | 1H), 7.67 (d, 1H), 7.79 (d, 1H), 7.91 (dd, 1H), 7.95 (bs, 1H) |
| 24 | MS 510 (M + 1) |
| 25 | MS 554 (M + 1) |
| 26 | 2.28 (s, 3H), 6.99 (d, 1H), 7.22 (d, 1H), 7.26 (d, 1H), 7.40 (dd, 1H), |
| | 7.61 (d, 1H), 7.86 (dd, 1H), 7.95 (d, 1H), 8.13 (d, 1H) |
| 27 | 2.28 (s, 3H), 6.99 (d, 1H), 7.22 (d, 1H), 7.27 (dd, 1H), 7.32 (d, 1H), |
| | 7.61 (d, 1H), 7.84 (d, 1H), 7.86 (dd, 1H), 8.12 (d, 1H) |
| 28 | 2.30 (s, 3H), 7.01 (d, 1H), 7.18 (d, 1H), 7.44 (d, 1H), 7.52 (d, 1H), |
| | 7.56 (dd, 1H), 7.76 (dd, 1H), 7.98 (d, 1H), 8.15 (d, 1H) |
| 30 | 2.29 (s, 3H), 7.00 (d, 1H), 7.22 (d, 1H), 7.23 (dd, 1H), 7.50 (d, 1H), |
| | 7.61 (d, 1H), 7.68 (d, 1H), 7.86 (dd, 1H), 8.12 (d, 1H) |
| 31 | 2.30 (s, 3H), 7.01 (d, 1H), 7.16-7.27 (m, 2H), 7.52 (d, 1H), 7.58 (d, 1H), |
| | 7.77 (m, 1H), 7.98 (d, 1H) |
| 32 | 2.26 (s, 3H), 3.74 (s, 3H), 6.86 (dd, 1H), 6.95 (d, 1H), 7.21 (d, 1H), |
| | 7.24 (d, 1H), 7.61 (d, 1H), 7.85 (dd, 1H), 8.11 (d, 1H) |
| 33 | 2.29 (s, 3H), 7.00-7.22 (m, 4H), 7.60-7.63 (d, 2H), 7.84-7.90 (m, 1H), |
| | 8.12 (s, 1H) |
| 34 | 2.29 (s, 3H), 7.01 (d, 1H), 7.22 (d, 1H), 7.33 (s, 1H), 7.48 (s, 1H), 7.61 (d, |
| | 1H), 7.76 (s, 1H), 7.85 (d, 1H), 7.92 (s, 1H), 8.13 (s, 1H) |
| 35 | 2.18-2.37 (m, 9H), 6.91-6.95 (d, 1H), 7.07 (s, 1H), 7.20-7.28 (m, 2H), |
| | 7.40 (s, 1H), 7.60 (d, 1H), 7.70 (m, 1H), 7.87 (m, 1H), 8.10 (s, 1H) |
| 36 | 2.27 (s, 3H), 2.71 (t, 2H), 2.93 (t, 2H), 6.95 (d, 1H), 7.01-7.05 (m, 1H), |
| | 7.04 (d, 1H), 7.1-7.2 (m, 2H), 7.26-7.35 (m, 5H), 7.59 (d, 1H), 7.93 (d, |
| | 1H), 8.33 (s, 1H), 8.56 (d, 1H). |
| 37 | MS 543 (M + 1) |
| 38 | MS 537 (M − 1) |
| 39 | 2.33 (s, 3H), 2.52 (s, 3H), 7.04-7.07 (m, 1H), 7.12 (d, 1H), 7.26 (d, 2H), |
| | 7.36 (dt, 1H), 7.43 (d, 1H), 7.56 (dd, 1H), 7.65 (d, 1H), 7.74 (dd, 1H) |
| 40 | 2.30 (s, 3H), 6.93 (d, 1H), 6.96 (d, 1H), 6.99 (d, 1H), 7.08 (m, 1H), |
| | 7.24-7.33 (m, 2H), 7.70 (d, 1H), 7.83 (dd, 1H), 8.17 (d, 1H) |
| 41 | MS 394 (M + 1) |
| 200 | 2.29 (s, 3H), 4.69 (s, 2H), 6.93 (d, 2H), 7.03 (dd, 1H), 7.23 (dd, 1H), |
| | 7.29 (d, 1H), 7.65 (d, 1H), 7.73 (d, 2H) |
| 201 | 2.24 (s, 3H), 3.82 (s, 2H), 6.56 (d, 2H), 7.01 (t, 1H), 7.22-7.29 (m, 2H), |
| | 7.52 (d, 2H), 7.67 (d, 1H). MS (esi): M + H⁺: 341 |
| 202 | 1.80 (t, 2H), 2.70 (m, 4H), 4.67 (s, 2H), 6.70 (d, 1H), 6.83 (dd, 1H), |
| | 7.03 (dd, 1H), 7.23 (dd, 1H), 7.30 (d, 1H), 7.67 (d, 1H), 7.94 (d, 1H), |
| | 12.27-12.33 (br. s, 1H). |
| 203 | 2.33 (s, 3H), 6.55 (d, 1H), 7.05 (dd, 1H), 7.22-7.35 (m, 2H), 7.58 (d, 1H), |
| | 7.69 (dd, 1H), 7.71 (d, 2H), 7.83 (d, 2H) |
| 204 | 1.47 (d, 3H), 1.79 (m, 2H), 2.69 (b, 4H), 4.85 (q, 1H), 6.64 (d, 1H), |
| | 6.78 (dd, 1H), 7.03 (m, 1H), 7.22 (m, 1H), 7.30 (d, 1H), 7.66 (d, 1H), 7.94 (d, 1H) |
| 205 | 2.83-2.89 (m, 2H), 2.97-3.02 (m, 2H), 4.68 (s, 2H), 6.86 (d, 1H), 6.88 (s, |
| | 1H), 7.02 (t, 1H), 7.23 (t, 1H), 7.31 (d, 1H), 7.51 (d, 1H), 7.67 (d, 1H) |
| 206 | 2.31 (s, 3H), 4.20 (d, 2H), 6.92-7.08 (m, 2H), 7.18-7.30 (m, 2H), 7.66 (d, |
| | 1H), 8.03 (dd, 1H), 8.40 (d, 1H), 8.53 (bt, 1H) |
| 207 | 0.86 (t, 3H), 1.25-1.45 (m, 4H), 2.83 (bt, 2H), 4.69 (s, 2H), 6.92 (d, 2H), |
| | 6.95-7.1 (m, 1H), 7.2-7.4 (m, 2H), 7.62 (d, 1H), 7.72 (d, 2H) |
| 208 | 1.80 (t, 2H), 2.70 (t, 4H), 3.26 (s, 3H), 4.77 (s, 2H), 6.70 (s, 1H), 6.81 (d, |
| | 1H), 7.04 (dd, 1H), 7.23 (dd, 1H), 7.29 (d, 1H), 7.66 (d, 1H), 7.98 (d, 1H). |
| 209 | 1.82 (t, 2H), 2.71 (t, 4H), 4.69 (s, 2H), 4.71 (s, 2H), 6.70 (s, 1H), 6.79 (d, |
| | 1H), 7.04 (dd, 1H), 7.23 (dd, 1H), 7.29 (m, 3H), 7.39 (m, 3H), 7.69 (d, |
| | 1H), 7.98 (d, 1H) |
| 210 | 2.24 (s, 3H), 3.86 (s, 2H), 7.05-7.10 (m, 1H), 7.24-7.30 (m, 1H), |
| | 7.32-7.40 (m, 3H), 7.70 (d, 1H), 7.77 (d, 2H) |
| 211 | 2.41 (s, 3H), 4.52 (s, 2H), 7.08-7.13 (t, 1H), 7.27-7.37 (m, 2H), 7.73 (d, |
| | 1H), 7.95 (d, 2H), 8.04 (d, 2H) |
| 212 | 2.31 (s, 3H), 7.04 (m, 1H), 7.23 (m, 1H), 7.27 (bd, 1H), 7.65 (bd, 1H), |
| | 7.79 (dd, 4H), 10.77 (s, 1H) |
| 300 | In CDCl₃, as a mixture of 2 isomers (2:1 ratio): 1.96 (s, 3 + 1 H), 2.34 (s, |
| | 2H), 6.93 (d, 1H), 7.01 (d, 1H), 7.08 (t, 1H), 7.26 (t, 1H), 7.37 (d, 1H), |
| | 7.59-7.64 (m, 2H), 7.87 (d, 1H), 8.05 (d, 1H), 8.34 (s, 1H) |
| 301 | 2.37 (s, 3H), 2.83 (s, 3H), 3.75 (s, 3H), 6.25 (d, 1H), 6.94 (dd, 1H), |
| | 7.14-7.20 (m, 2H), 7.26-7.43 (m, 4H), 7.63 (d, 1H), 8.9 (bs, 1H) |
| 302 | 2.28 (s, 3H), 3.62 (s, 2H), 6.95 (d, 1H), 7.00-7.08 (m, 2H), 7.17-7.27 (m, |
| | 3H), 7.39 (dd, 1H), 7.62-7.70 (m, 3H) |
| 303 | 2.31 (s, 3H), 3.86 (s, 3H), 6.30 (d, 1H), 6.63 (d, 1H), 7.01 (m, 1H), 7.24 (d, |
| | 1H), 7.42 (br. s, 2H), 7.52 (br. s, 1H), 7.73 (2 × d, 4H), 7.82-7.86 (m, 2H) |
| 304 | 2.3 (s, 3H), 2.54 (t, 2H), 2.71 (t, 2H), 7.01-7.15 (m, 7), 7.19-7.24 (m, 2H), |
| | 7.27 (s, 1H), 7.63 (d, 1H), 7.69 (t, 1H), 7.81 (d, 1H), 8.06 (d, 1H), 8.17 (s, |
| | 1H), 11.8 (s, 1H), 12.2 (s, 1H). MS (-esi): M − H⁻: 543.3 |
| 305 | 1.65-1.70 (m, 2H), 2.23 (t, 2H), 2.29 (s, 3H), 2.40 (t, 2H), 7.00-7.26 (m, |
| | 9H), 7.64 (d, 1H), 7.69 (t, 1H), 7.81 (d, 1H), 8.05 (d, 1H), 8.20 (s, 1H), |
| | 11.75 (s, 1H), 12.18 (s, 1H). MS (-esi):: M − H⁻: 557.7 |
| 306 | Obtained as a 2:1 mixture of isomers. 1.87 (s, 2H), 1.92 (s, 2H), 2.41 (s, |
| | 2H), 7.01-7.06 (m, 1H), 7.22 (s, 2H), 7.44 (d, 1H), 7.59 (d, 1H), |
| | 7.64-7.69 (m, 2H), 7.80 (d, 1H), 8.04 (d, 2H). MS (-esi): M − H⁻: 469.2 |
| 307 | 2.29 (s, 3H), 7.00 (d, 1H), 7.06 (t, 1H), 7.22-7.27 m, 3H), 7.44 (t, 2H), |
| | 7.59 (t, 1H), 7.67 (t, 1H), 7.73 (d, 1H), 7.85-7.97 (m, 3H), 8.07 (d, 1H), 8.29 (s, |
| | 1H), 11.5 (br s, 1H), 12.2 (br s, 1H). MS (-esi): M − H⁻: 515.3 |
| 308 | 2.29 (s, 3H), 3.54 (s, 2H), 7.00 (d, 1H), 7.05-7.30 (m, 9H), 7.65 (br s, 1H), |
| | 7.67 (t, 1H), 7.79 (d, 1H), 8.04 (d, 1H), 8.17 (s, 1H), 11.7 (br s, 1H), |
| | 12.49 (s, 1H). MS (-esi): M − H⁻: 529.3 |
| 309 | 1.87 (s, 3H), 5.07 (s, 1H), 7.01 (d, 1H), 7.03-7.09 (m, 5H), 7.16-7.30 (m, |
| | 9H), 7.64 (d, 1H), 7.69 (d, 1H), 7.76 (s, 1H), 8.07 (d, 1H), 8.14 (s, 1H), |
| | 11.82 (s, 1H), 12.70 (s, 1H) MS (-esi): M − H⁻: 605.3 |
| 310 | MS 505 (M + 1) |
| 311 | MS 489 (M + 1) |
| 312 | 2.31 (s, 3H), 7.04-7.08 (m, 2H), 7.23-7.28 (m, 2H), 7.22 (d, 1H), 7.37 (d, |
| | 1H), 7.42-7.47 (m, 2H), 7.54 (t, 1H), 7.65 (d, 1H), 7.70-7.75 (m, 2H), |
| | 7.83 (d, 1H), 7.88-7.95 (m, 2H), 8.09 (d, 1H), 8.20 (s, 1H), 8.33 (s, 1H) |
| 313 | Obtained as a 3:2 mixture of isomers. 1.87 (s, 1.2H), 2.30 (s, 1.8H), |
| | 7.02 (d, 1H), 7.06 (t, 1H), 7.22-7.32 (m, 2H), 7.33-7.47 (m, 3H), 7.64-7.81 (m, |
| | 6H), 7.91-7.99 (m, 3H), 8.08 (d, 1H), 8.31 (s, 1H) |
| 314 | 2.29 (s, 3H), 4.79 (br. s, 2H), 6.97 (d, 1H), 7.03 (dd, 1H), 7.16 (d, 1H), |
| | 7.24 (dd, 1H), 7.33-7.41 (m, 4H), 7.42 (m, 2H), 7.57 (d, 2H), 7.78 (s, 1H), |
| | 7.80 (d, 1H). |
| 315 | 2.28 (s, 3H), 3.03 (t, 2H), 3.51 (t, 2H), 6.97 (d, 1H), 7.06 (m, 1H), |
| | 7.12-7.26 (br. m, 8H), 7.55-7.62 (m, 2H), 7.78 (d, 1H), 7.87 (s, 1H). |
| 316 | 2.09 (tt, 2H), 2.28 (s, 3H), 2.74 (t, 2H), 3.52 (t, 2H), 6.98 (d, 1H), 7.05 (dd, |
| | 1H), 7.14-7.26 (m, 8H), 7.62-7.65 (m, 2H), 7.86 (d, 1H), 7.91 (s, 1H). |
| 317 | In CDCl₃: 2.43 (s, 3H), 3.96 (s, 2H), 4.55 (s, 2H), 6.52 (bs, 2H), 6.89 (d, |
| | 1H), 6.93 (d, 1H), 7.21-7.46 (m, 7H), 7.52-7.72 (m, 3H), 7.96 (d, 2H), |
| | 8.39 (s, 1H). MS (esi): M + H⁺: 561 |
| 318 | As a mixture of 2 isomers (1:1 ratio): 2.29 (s, 1.5H), 2.46 (s, 1.5H), 2.68 (t, |
| | 2H), 4.06 (t, 2H), 6.74 (t, 2H), 6.84 (t, 1H), 7.00 (d, 1H), 7.12-7.30 (m, |
| | 3H), 7.34 (d, 1H), 7.44 (s, 1H), 7.58 (d, 1H), 7.61-7.75 (m, 2H), 7.86 (dd, |
| | 1H), 8.09 (dd, 1H), 8.23 (d, 1H). MS (esi): M + H⁺: 561 |
| 319 | 2.30 (s, 3H), 2.56 (t, 2H), 3.03 (t, 2H), 7.00 (d, 1H), 7.03-7.17 (m, 2H), |
| | 7.18-7.30 (m, 7H), 7.62-7.73 (m, 2H), 7.81 (d, 1H), 8.06 (d, 1H), 8.20 (s, |
| | 1H). MS (esi): M + H⁺: 577 |
| 320 | 1.91 (m, 2H), 2.34 (m, 2H), 2.50 (m, 1H), 6.98 (d, 1H), 7.03 (d, 3H), |
| | 7.13 (m, 2H), 7.18 (d, 1H), 7.24 (m, 2H), 7.57 (t, 1H), 7.65 (bd, 1H), 7.80 (d, |
| | 1H), 7.90 (d, 1H), 7.97 (bs, 1H), 8.19 (s, 1H). MS (esi): M + H⁺: 574 |
| 321 | 1.39 (m, 4H), 2.23 (t, 2H), 2.29 (s, 3H), 2.41 (t, 2H), 7.01 (t, 2H), 7.08 (t, |
| | 2H), 7.17 (t, 2H), 7.21-7.31 (m, 3H), 7.44 (s, 1H), 7.67 (t, 2H), 7.80 (d, |
| | 1H), 8.05 (d, 1H), 8.19 (s, 1H). MS (esi): M + H⁺: 573 |
| 322 | 1.10 (m, 2H), 1.40 (m, 4H), 2.21 (t, 2H), 2.29 (s, 3H), 2.38 (t, 2H), |
| | 6.99-7.11 (m, 5H), 7.13-7.31 (m, 5H), 7.65 (d, 1H), 7.69 (t, 1H), 7.82 (d, 1H), |
| | 8.06 (d, 1H), 8.19 (s, 1H). MS (esi): M + H⁺: 587 |
| 323 | 2.30 (s, 3H), 3.17 (d, 2H), 6.16 (m, 1H), 6.38 (d, 1H), 7.00 (d, 1H), 7.05 (t, |
| | 1H), 7.14-7.34 (m, 8H), 7.69 (m, 2H), 7.83 (d, 1H), 8.06 (d, 1H), 8.21 (s, |
| | 1H). MS (esi): M + H⁺: 557 |
| 324 | 1.64 (m, 2H), 2.20 (t, 2H), 2.29 (s, 3H), 2.33 (t, 2H), 3.64 (s, 3H), 6.72 (d, |
| | 2H), 6.91 (d, 2H), 7.00 (d, 1H), 7.05 (t, 1H), 7.23 (d, 1H), 7.27 (t, 2H), |
| | 7.65 (d, 1H), 7.70 (t, 1H), 7.81 (m, 1H), 8.06 (d, 1H), 8.20 (s, 1H). MS |
| | (esi): M + H⁺: 589 |
| 325 | MS (esi): M + H⁺: 685 |
| 326 | MS (esi): M + H⁺: 619 |
| 327 | MS (esi): M + H⁺: 619 |
| 328 | MS (esi): M + H⁺: 604 |
| 329 | 1.21 (s, 9H), 2.30 (s, 3H), 2.35 (m, 2H), 2.52 (m, 2H), 3.84 (m, 1H), |
| | 6.68 (d, 1H), 6.98-7.27 (m, 9H), 7.67 (t, 2H), 7.80 (d, 1H), 8.03 (d, 1H), 8.19 (s, |
| | 1H). MS (esi): M + H⁺: 674 |
| 330 | 1.21 (s, 9H), 2.30 (s, 3H), 2.35 (m, 2H), 2.56 (m, 2H), 3.84 (m, 1H), |
| | 6.68 (d, 1H), 6.98-7.31 (m, 9H), 7.67 (t, 2H), 7.81 (d, 1H), 8.03 (d, 1H), 8.19 (s, |
| | 1H) |
| 331 | 2.34 (s, 3H), 2.55 (m, 1H), 2.71 (m, 2H), 2.86 (m, 2H), 7.05 (d, 1H), |
| | 7.10 (t, 2H), 7.20-7.33 (m, 5H), 7.70 (m, 1H), 7.78 (d, 1H), 7.86 (m, 3H), |
| | 8.12 (d, 1H), 8.22 (s, 1H). MS (esi): M + H⁺: 574 |
| 332 | 7.76 (d, J = 6.81 Hz, 1H), 7.70 (d, J = 7.71 Hz, 1H), 7.62 (t, J = 1.59 Hz, |
| | 1H), 7.52-7.49 (m, 2H), 7.47-1.43 (m, 1H), 7.28-7.26 (m, 3H), |
| | 7.10-7.05 (m, 1H), 4.77 (s, 2H), 2.37 (s, 3H), 2.04 (s, 3H). MS (+ESI): M + H+: 485.1. |
| 333 | MS (+ESI): M + H+: 547.1. |
| 334 | MS (+ESI): M + H+: 561.2. |
| 335 | 11.85 (br s, 2H), 7.75 (d, J = 7.77 Hz, 1H), 7.61-7.58 (m, 2H), |
| | 7.48-7.43 (m, 2H), 7.41 (t, J = 7.83 Hz, 1H), 7.35-7.27 (m, 7H), 7.13 (d, J = 7.98 Hz, |
| | 1H), 7.08-7.03 (m, 1H), 4.77 (s, 2H), 3.62 (s, 2H), 2.38 (s, 3H). MS |
| | (+ESI): M + H+: 575.1. |
| 336 | 11.85 (br s, 1H), 11.56 (br s, 1H), 7.75 (d, J = 8.28 Hz, 1H), 7.66 (d, J = 7.74 Hz, |
| | 1H), 7.60 (s, 1H), 7.48 (d, J = 3.96 Hz, 1H), 7.45 (d, J = 7.77 Hz, |
| | 1H), 7.41 (d, J = 4.87 Hz, 1H), 7.29-7.22 (m, 5H), 7.18-7.13 (m, 3H), |
| | 7.09-7.03 (m, 1H), 4.78 (s, 2H), 2.55 (t, J = 7.32 Hz, 2H), 2.38 (s, 3H), 2.25 (t, J = 7.35 Hz, |
| | 2H), 1.84 (q, J = 7.74 Hz, 2H). MS (+ESI): M + H+: 589.2. |
| 339 | MS 574 (M + 1) |
| 340 | 2.27 (s, 3H), 3.14 (dd, 1H), 3.45 (dd, 1H), 3.66 (bm, 1H), 6.98 (d, 1H), |
| | 7.04 (t, 1H), 7.13 (d, 1H), 7.18-7.38 (m, 7H), 7.56 (t, 1H), 7.65 (d, 1H), |
| | 7.89 (d, 1H), 8.10 (bm, 2H), 8.16 (s, 1H) |
| 341 | 1.64 (m, 2H), 2.17 (s, 3H), 2.20 (t, 2H), 2.34 (s, 3H), 2.46 (t, 2H), |
| | 6.85-7.08 (m, 5H), 7.21-7.28 (m, 3H), 7.62-7.81 (m, 4H), 8.05 (d, 1H), 8.20 (s, |
| | 1H), MS 573 (M + 1) |
| 342 | MS 483 (M + 1) |
| 343 | 1.72 (m, 2H), 2.23 (t, 2H), 2.30 (s, 3H), 2.46 (t, 2H), 6.98-8.22 (m, 19H), |
| | MS 635 (M + 1) |
| 344 | MS 574 (M + 1) |
| 345 | MS 498 (M + 1) |
| 346 | MS 498 (M + 1) |
| 347 | MS 471 (M + 1) |
| 348 | MS 547 (M + 1) |
| 349 | MS 607 (M + 1) |
| 350 | MS 573 (M + 1) |
| 351 | 1.72 (m, 2H), 2.27 (t, 2H), 2.30 (s, 3H), 2.42 (t, 2H), 7.01-8.27 (m, 19H), |
| | MS 635 (M + 1) |
| 352 | 1.71 (m, 2H), 2.29 (t, 2H), 2.34 (s, 3H), 2.45 (t, 2H), 7.03 (d, 1H), 7.08 (m, |
| | 2H), 7.18 (t, 1H), 7.22 (d, 1H), 7.26-7.36 (m, 3H), 7.47 (s, 1H), |
| | 7.65-7.78 (m, 2H), 7.85 (d, 1H), 8.10 (d, 1H), 8.24 (s, 1H), MS 639 (M + 1) |
| 353 | 1.65 (m, 2H), 2.23 (t, 2H), 2.35 (t, 2H), 2.36 (s, 3H), 4.16 (t, 4H), 6.48 (dd, |
| | 1H), 6.55 (d, 1H), 6.66 (d, 1H), 7.03 (d, 1H), 7.09 (t, 1H), 7.24-7.34 (m, |
| | 3H), 7.65-7.78 (m, 2H), 7.85 (d, 1H), 8.09 (d, 1H), 8.22 (s, 1H), MS |
| | 617 (M + 1) |
| 354 | 2.65 (s, 3H), 2.55 (t, 2H), 3.03 (t, 2H), 7.03 (t, 1H), 7.11-7.16 (m, 1H), |
| | 7.20-7.27 (m, 6H), 7.43 (d, 1H), 7.58 (d, 1H), 7.64-7.71 (m, 2H), 7.81 (dt, |
| | 1H), 8.03-8.08 (m, 2H), 11.83 (br. s., 1H), 12.21 (s, 1H). MS 591.3 (M + 1) |
| 355 | 1.94 (s, 3H), 2.31 (s, 3H), 7.4 (d, 1H), 7.06 (td, 1H), 7.24 (td, 1H), |
| | 7.26-7.27 (m, 1H), 7.38 (d, 1H), 7.62 (d, 1H), 7.81-7.82 (m, 2H), 8.39-8.40 (m, |
| | 1H). MS 489.1 (M + 1) |
| 356 | 1.91 (s, 3H), 2.30 (s, 3H), 2.60 (s, 3H), 6.99 (d, 1H), 7.02 (d, 1H), |
| | 7.04-7.07 (m, 1H), 7.24 (td, 1H), 7.28 (d, 1H), 7.55 (d, 1H), 7.63 (d, 1H), |
| | 7.74 (dd, 1H), 8.20 (d, 1H). MS 467.1 (M + 1) |
| 357 | 2.29 (s, 3H), 2.55 (t, 2H), 2.60 (s, 3H), 3.02 (t, 2H), 6.99 (d, 1H), 7.02 (d, |
| | 1H), 7.02-7.07 (m, 1H), 7.11-7.16 (m, 1H), 7.18-7.30 (m, 6H), 7.55 (d, |
| | 1H), 7.62 (d, 1H), 7.74 (dd, 1H), 8.20 (d, 1H), 11.81 (s, 1H), 12.16 (s, 1H). |
| | MS 589.2 (M + 1) |
| 358 | 1.97 (s, 3H), 2.29 (s, 3H), 7.01 (d, 1H), 7.02-7.08 (m, 1H), 7.22-7.28 (m, |
| | 2H), 7.29 (d, 1H), 7.64 (d, 1H), 7.75 (d, 1H), 8.02 (dd, 1H), 8.35 (d, 1H), |
| | 12.55 (s, 1H). MS 487.3 (M + 1) |
| 359 | 2.29 (s, 3H), 2.60 (t, 2H), 3.03 (t, 2H), 7.01 (d, 1H), 7.02-7.08 (m, 1H), |
| | 7.14-7.19 (m, 1H), 7.23-7.30 (m, 7H), 7.64 (d, 1H), 7.75 (d, 1H), 8.03 (dd, |
| | 1H), 8.37 (d, 1H), 11.82 (s, 1H), 12.63 (s, 1H). MS 609.2 (M + 1) |
| 360 | 1.27 (s, 9H), 2.29 (s, 3H), 2.53 (m, 1H), 2.69 (s, 5H), 4.13 (m, 1H), 6.98 (d, |
| | 1H), 7.02-7.07 (m, 1H), 7.14-7.29 (m, 8H), 7.64-7.69 (m, 2H), 7.79 (d, 2, |
| | 1H), 8.03 (d, 1H), 8.20 (s, 1H), 11.81 (s, 1H), 12.45 (s, 1H). MS |
| | 718.4 (M + 1) |
| 361 | 2.29 (s, 3H), 2.66-2.73 (m, 4H), 2.81-3.00 (m, 2H), 3.84 (m, 1H), 6.99 (d, |
| | 1H), 7.05-7.07 (m, 1H), 7.14-7.27 (m, 9H), 7.52-7.67 (m, 3H), 7.80 (d, |
| | 1H), 7.94 (d, 1H), 8.05 (br s, 3H), 8.20 (s, 1H). MS 618.9 (M + 1) |
| 362 | 0.82 (d, 6H), 2.29 (d, 2H), 2.33 (s, 3H), 2.56 (m, 4H), 7.03 (d, 1H), 7.09 (t, |
| | 1H), 7.25-7.35 (m, 3H), 7.65-7.78 (m, 2H), 7.85 (d, 1H), 8.09 (d, 1H), |
| | 8.24 (s, 1H), MS 557 (M + 1) |
| 363 | 0.83 (t, 3H), 1.45 (m, 2H), 2.34 (s, 3H), 2.37 (t, 2H), 2.55 (m, 4H), 7.04 (d, |
| | 1H), 7.09 (t, 1H), 7.27-7.35 (m, 3H), 7.65-7.78 (m, 2H), 7.85 (d, 1H), |
| | 8.09 (d, 1H), 8.24 (s, 1H), MS 543 (M + 1) |
| 364 | 1.08 (d, 6H), 2.34 (s, 3H), 2.58 (m, 4H), 2.80 (m, 1H), 7.04 (d, 1H), 7.09 (t, |
| | 1H), 7.26-7.34 (m, 3H), 7.65-7.78 (m, 2H), 7.85 (d, 1H), 8.09 (d, 1H), |
| | 8.24 (s, 1H), MS 543 (M + 1) |
| 365 | 1.07 (t, 3H), 2.34 (s, 3H), 2.41 (q, 2H), 2.59 (m, 4H), 7.04 (d, 1H), 7.09 (t, |
| | 1H), 7.25-7.35 (m, 3H), 7.63-7.78 (m, 2H), 7.87 (d, 1H), 8.09 (d, 1H), |
| | 8.24 (s, 1H), MS 529 (M + 1) |
| 366 | 0.75 (t, 3H), 1.15 (m, 2H), 1.40 (m, 2H), 2.25 (t, 2H), 2.34 (s, 3H), 7.04 (d, |
| | 1H), 7.09 (t, 1H), 7.25-7.33 (m, 3H), 7.65-7.78 (m, 2H), 7.84 (d, 1H), |
| | 8.09 (d, 1H), 8.23 (s, 1H), MS 497 (M + 1) |
| 367 | 0.76 (d, 6H), 0.84 (m, 1H), 1.33 (m, 2H), 2.25 (t, 2H), 2.34 (s, 3H), 7.04 (d, |
| | 1H), 7.09 (t, 1H), 7.23-7.33 (m, 3H), 7.65-7.78 (m, 2H), 7.84 (d, 1H), |
| | 8.08 (d, 1H), 8.23 (s, 1H), MS 511 (M + 1) |
| 368 | 0.77 (d, 6H), 1.92 (m, 1H), 2.13 (d, 2H), 2.34 (s, 3H), 7.03 (d, 1H), 7.10 (t, |
| | 1H), 7.21-7.36 (m, 2H), 7.45 (d, 1H), 7.65-7.78 (m, 2H), 7.84 (d, 1H), |
| | 8.10 (d, 1H), 8.23 (s, 1H), MS 497 (M + 1) |
| 369 | 2.34 (s, 3H), 2.55 (t, 2H), 2.94 (t, 2H), 3.72 (s, 3H), 6.86 (d, 2H), 7.04 (d, |
| | 1H), 7.09 (t, 1H), 7.20-7.48 (m, 5H), 7.62-7.80 (m, 2H), 7.85 (d, 1H), |
| | 8.10 (d, 1H), 8.24 (s, 1H), MS 607 (M + 1) |
| 370 | 1.05 (d, 3H), 1.11 (d, 3H), 2.34 (s, 3H), 2.77-2.99 (m, 3H), 3.86 (bs, 1H), |
| | 7.03 (d, 1H), 7.09 (t, 1H), 7.21 (d, 1H), 7.24-7.38 (m, 2H), 7.62-7.75 (m, |
| | 2H), 7.85 (d, 1H), 8.00 (d, 1H), 8.08 (bs, 2H), 8.23 (s, 1H), MS 558 (M + 1) |
| 371 | 2.34 (s, 3H), 2.65 (t, 2H), 2.92 (m, 2H), 7.04 (d, 1H), 7.10 (t, 1H), |
| | 7.24-7.35 (m, 3H), 7.60-7.72 (bm, 3H), 7.75 (t, 1H), 7.88 (d, 1H), 8.12 (d, 1H), |
| | 8.26 (s, 1H), MS 484 (M + 1) |
| 372 | 2.34 (s, 3H), 3.32 (dd, 1H), 3.57 (dd, 1H), 4.01 (bs, 1H), 7.03-7.18 (m, |
| | 3H), 7.22-7.35 (m, 4H), 7.51-7.74 (m, 4H), 7.78-7.87 (m, 2H), 7.95 (d, |
| | 1H), 8.08 (d, 1H), 8.11-8.28 (m, 4H), MS 610 (M + 1) |
| 373 | MS 606 (M + 1) |
| 374 | 1.99 (m, 2H), 2.34 (s, 3H), 2.36 (m, 2H), 3.65 (s, 2H), 3.80 (bs, 1H), |
| | 7.03 (d, 1H), 7.09 (t, 1H), 7.20-7.37 (m, 8H), 7.63-7.72 (m, 2H), 7.83 (d, 2H), |
| | 7.99-8.12 (m, 3H), 8.25 (s, 1H), MS 620 (M + 1) |
| 375 | 1.56 (m, 2H), 2.14 (t, 2H), 2.34 (s, 3H), 2.43 (t, 2H), 7.03 (d, 1H), |
| | 7.05-7.41 (m, 10H), 7.47 (d, 2H), 7.65-7.83 (m, 4H), 8.09 (d, 1H), 8.21 (s, 1H), MS |
| | 635 (M + 1) |
| 376 | 2.34 (s, 3H), 2.88 (bs, 4H), 7.04-7.42 (m, 10H), 7.47 (d, 2H), 7.64-7.82 (m, |
| | 4H), 7.95 (d, 1H), 8.12 (d, 1H), 8.34 (s, 1H), MS 621 (M + 1) |
| 377 | 2.34 (s, 3H), 7.04 (d, 1H), 7.10 (t, 1H), 7.25-7.85 (m, 15H), 7.98 (d, 1H), |
| | 8.10-8.20 (m, 2H), 8.36 (s, 1H), MS 619 (M + 1) |
| 378 | 2.34 (s, 3H), 3.97 (s, 2H), 7.05 (d, 1H), 7.10 (t, 1H), 7.18-7.34 (m, 7H), |
| | 7.38-7.50 (m, 3H), 7.67-7.80 (m, 4H), 7.93 (d, 1H), 8.10 (d, 1H), 8.33 (s, |
| | 1H), MS 607 (M + 1) |
| 379 | 2.34 (s, 3H), 3.99 (s, 2H), 7.04 (d, 1H), 7.10 (t, 1H), 7.15-7.37 (m, 9H), |
| | 7.47 (d, 1H), 7.63-7.88 (4H), 7.92 (d, 1H), 8.10 (d, 1H), 8.32 (s, 1H), MS |
| | 607 (M + 1) |
| 380 | 2.34 (s, 3H), 3.05 (m, 2H), 4.00 (bs, 1H), 7.02-7.37 (m, 9H), 7.65-7.76 (m, |
| | 2H), 7.91 (d, 1H), 8.00-8.19 (m, 4H, 8.24 (s, 1H), MS 560 (M + 1) |
| 381 | MS 590 (M + 1) |
| 383 | 0.83 (d, 6H), 1.68 (m, 1H), 2.34 (s, 3H), 2.35 (m, 2H), 3.90 (bs, 1H), |
| | 7.04 (d, 1H), 7.09 (t, 1H), 7.22 (d, 1H), 7.25-7.35 (m, 2H), 7.62-7.73 (m, 2H), |
| | 7.88 (d, 1H), 7.99-8.25 (m, 4H), MS 572 (M + 1) |
| 384 | 0.84 (t, 3H), 1.45 (m, 2H), 2.34 (s, 3H), 2.40 (m, 2H), 2.90 (m, 2H), |
| | 3.85 (bs, 1H), 7.03 (d, 1H), 7.09 (t, 1H), 7.21 (d, 1H), 7.23-7.38 (m, 2H), |
| | 7.47 (d, 1H), 7.61-7.80 (m, 2H), 7.85 (d, 1H), 7.99 (d, 1H), 8.07 (bs, 2H), |
| | 8.23 (s, 1H), MS 558 (M + 1) |
| 385 | 1.08 (t, 3H), 2.34 (s, 3H), 2.41 (m, 2H), 2.90 (m, 2H), 3.88 (bs, 1H), |
| | 7.03 (d, 1H), 7.09 (t, 1H), 7.22 (d, 1H), 7.24-7.37 (m, 2H), 7.62-7.73 (m, 2H), |
| | 7.85 (d, 1H), 8.01 (d, 1H), 8.10 (bs, 2H), 8.23 (s, 1H), MS 544 (M + 1) |
| 386 | 1.34 (d, 3H), 2.34 (s, 3H), 3.84 (bs, 1H), 7.04 (d, 1H), 7.10 (t, 1H), |
| | 7.24-7.35 (m, 3H), 7.67-7.78 (m, 2H), 7.88 (d, 1H), 7.99-8.15 (m, 3H), 8.25 (s, |
| | 1H), MS 484 (M + 1) |
| 387 | 2.30 (s, 3H), 7.00 (d, 1H), 7.04-7.10 (m, 2H), 7.23-7.30 (m, 2H), 7.40 (t, |
| | 1H), 7.52-7.55 (m, 1H), 7.69-7.73 (m, 2H), 7.96 (t, 1H) |
| 388 | (CDCl₃) 2.46 (s, 3H), 6.88 (d, 1H), 6.98 (s, 1H), 7.34 (t, 1H), 7.47 (t, 1H), |
| | 7.55-7.62 (m, 2H), 7.74-7.88 (m, 3H), 7.93-8.00 (m, 2H), 8.25 (t, 1H), |
| | 8.43 (bs, 1H) |
| 389 | 2.30 (s, 3H), 6.85 (d, 1H), 7.02 (t, 1H), 7.0-7.1 (m, 1H), 7.20 (d, 1H), |
| | 7.2-7.3 (m, 2H), 7.64 (t, 1H), 7.65-7.7 (m, 1H), 7.85-7.89 (m, 1H), |
| | 7.96-8.00 (m, 1H), 8.29 (t, 1H), 8.49 (d, 2H), MS 491 (M + 1) |
| 390 | 2.26 (s, 3H), 6.98 (d, 1H), 7.05 (t, 1H), 7.18-7.39 (m, 6H), 7.63 (t, 2H), |
| | 7.72 (d, 1H), 7.75-7.79 (m, 1H), 7.96-8.00 (m, 1H), 8.18 (t, 1H) |
| 400 | 2.39 (s, 3H), 7.05 (dd, 1H), 7.19 (d, 1H), 7.21-7.35 (m, 3H), 7.52 (dd, 1H), |
| | 7.67 (dd, 1H), 7.76-7.86 (m, 2H), 8.13 (s, 1H) |
| 401 | 2.39 (s, 3H), 7.05 (dd, 1H), 7.22-7.37 (m, 2H), 7.49 (dd, 1H), 7.61 (d, 1H), |
| | 7.69-7.74 (m, 3H), 7.80 (d, 1H), 8.08 (s, 1H) |
| 500 | Obtained as a 1:1 mixture of isomers. 2.41 (s, 1.5H), 2.47 (s, 1.5H), 7.05 (t, |
| | 1H), 7.24 (t, 1H), 7.31 (d, 1H), 7.50 (t, 1H), 7.62 (t, 1H), 7.67 (d, 2H), |
| | 7.83 (d, 1H), 7.94 (d, 2H), 8.05 (s, 1H), 8.21 (s, 1H), 11.5 (br s, 1H) |
| 501 | 2.34 (s, 3H), 2.53 (s, 3H), 7.01-7.11 (m, 2H), 7.27 (dd, 1H), 7.50 (dd, 1H), |
| | 7.67 (d, 1H), 7.73 (d, 1H), 8.23 (d, 1H), 8.62 (s, 1H) |
| 502 | 2.39 (s, 3H), 7.08 (m, 1H), 7.27 (m, 2H), 7.48 (d, 1H), 7.75 (d, 2H), |
| | 7.87 (d, 1H), 7.92 (d, 1H), 8.02 (t, 1H), 8.17 (d, 1H), MS 395 (M + 1) |
| 503 | 1.92 (s, 3H), 2.41 (s, 3H), 7.04 (t, 1H), 7.23 (t, 1H), 7.40 (s, 1H), 7.56 (t, |
| | 1H), 7.62-7.77 (m, 3H), 7.82-7.95 (m, 2H), 8.00-8.07 (m, 2H), 8.13 (s, |
| | 1H), MS 465 (M + 1) |
| 600 | 2.46 (s, 3H), 7.09 (t, 1H), 7.28 (t, 1H), 7.36 (d, 1H), 7.59 (t, 1H), 7.72 (d, |
| | 1H), 7.96 (d, 1H), 8.03 (d, 1H), 8.11 (t, 1H), 8.19 (d, 1H), 8.26 (d, 1H), |
| | 8.55 (s, 1H), MS 389 (M + 1) |
| 601 | MS 570 (M + 1) |
| 602 | 1.40 (t, 3H), 2.46 (s, 3H), 4.42 (q, 2H), 7.10 (t, 1H), 7.29 (t, 1H), 7.37 (bs, |
| | 1H), 7.61 (t, 1H), 7.70 (bs, 1H), 7.95 (d, 1H), 8.06 (d, 1H), 8.16 (t, 2H), |
| | 8.28 (d, 1H), 8.58 (s, 1H), MS 417 (M + 1) |
| 603 | MS 502 (M + 1) |
| 604 | MS 547 (M + 1) |
| 605 | 2.45 (s, 3H), 3.77 (m, 2H), 4.39 (m, 2H), 4.95 (t, 1H), 7.08 (t, 1H), 7.28 (t, |
| | 1H), 7.36 (bs, 1H), 7.60 (t, 1H), 7.72 (bs, 1H), 7.95 (d, 1H), 8.07-8.18 (m, |
| | 3H), 8.28 (d, 1H), 8.56 (s, 1H) |
| 606 | 2.98 (m, 2H), 3.13 (m, 2H), 7.08 (t, 1H), 7.28 (t, 1H), 7.38 (d, 2H), 7.51 (d, |
| | 2H), 7.65 (t, 1H), 7.73 (t, 1H), 7.86 (s, 1H), 7.97 (t, 1H), 8.20 (s, 1H) |
| 607 | 2.51 (s, 3H), 7.10 (t, 1H), 7.29 (t, 1H), 7.31 (br s, 1H), 7.63 (t, 1H), |
| | 7.73 (br t, 1H), 8.01 (d, 1H), 8.19-8.26 (m, 3H), 8.37 (dt, 1H), 8.58 (d, 1H). MS |
| | (-ESI): 411.3 (M − 1) |
| 608 | MS 466 (M + 1) |
| 609 | 2.4 (s, 3H), 7.09 (t, 1H), 7.29 (t, 1H), 7.34 (br s, 1H), 7.64 (t, 1H), 7.71 (m, |
| | 1H), 7.98 (d, 1H), 8.17-8.27 (m, 2H), 8.37 (d, 1H), 8.52 (d, 1H), 8.62 (s, |
| | 1H), 12.2 (s, 1H). MS 454.3 (M + 1) |
| 610 | 3.00 (m, 2H), 3.17 (m, 2H), 7.09 (t, 1H), 7.29 (t, 1H), 7.38 (d, 1H), 7.55 (d, |
| | 1H), 7.55 (d, 1H), 8.00-8.23 (m, 4H), 8.36 (s, 1H), MS 401 (M + 1) |
| 611 | 1.90 (m, 2H); 2.83 (m, 4H); 7.10 (t, 1H); 7.28 (t, 1H); 7.37 (d, 2H); 7.75 (d, |
| | 1H); 8.02 (dd, 1H); 8.05-8.18 (m, 3H), 8.81 (s, 1H), MS 415 (M + 1) |
| 612 | 3.00 (t, 2H); 4.32 (t, 2H); 7.00-7.09 (m, 2H); 7.23 (d, 1H); 7.67 (d, 1H); |
| | 7.95 (m, 1H); 8.02-8.10, (m, 3H); 8.70 (s, 1H), MS 417 (M + 1) |
| 613 | Includes some benzothiazole starting material impurities. 1.99 (s, 3H), |
| | 2.43 (s, 3H), 7.05 (br t, J = 7.8 Hz, 1H), 7.25 (br t, J = 7.5 Hz, 1H), |
| | 7.29-7.26 (m, 1H), 7.59 (t, J = 8.0 Hz, 1H), 7.65-7.62 (m, 1H), 7.96 (d, J = 8.5 Hz, |
| | 1H), 8.02 (dd, J = 7.5 and 1 Hz, 1H), 8.06 (d, J = 7.8 Hz, 1H), 8.22 (t, J = 7.6 Hz, |
| | 1H), 8.29 (dd, J = 9.1 and 1.0 Hz, 1H), 8.40 (t, J = 1.7 Hz, 1H). |
| | MS: M + H = 466.2. |
| 614 | 1.94-1.88 (m, 2H), 2.84-2.81 (m, 4H), 7.08 (d t, J = 7.8 and 1.2 Hz, 1H), |
| | 7.28 (d t, J = 7.23 and 1.0 Hz, 1H), 7.38-7.32 (m, 2H), 7.60 (dd, J = 7.9 and |
| | 2 Hz, 1H), 7.66 (t, J = 7.74 Hz, 1H), 7.72 (d, J = 7.6 Hz, 1H), 7.92 (d q, J = 8.5 |
| | and 1.1 Hz, 1H), 7.97 (d t, J = 7.9 and 1.2 Hz, 1H), 8.23 (t, J = 1.6 Hz, |
| | 1H), 8.35 (d, J = 2.0 Hz, 1H), 12.4 (br s, 1H). LCMS: M + H = 414.2. |
| 615 | 1.86-1.91 (m, 2H), 2.03 (s, 3H), 2.81-2.84 (m, 4H), 7.05-7.11 (m, 1H), |
| | 7.28 (t, J = 7.41 Hz, 1H), 7.35 (br s, 1H), 7.39 (d, J = 8.01, 1H), 7.68 (br s., 1H), |
| | 7.94 (dd, J = 7.9 and 1.7, 1H), 8.05 (d, J = 7.4 Hz, 1H), 8.23-8.17 (m, 2H), |
| | 8.64 (s, 1H), 11.62 (br s, 1H), 12.37 (br s, 1H). LCMS: M + H = 492.2 (99.8% |
| | purity). |
| 616 | 1.00-1.08 (m, 2H), 1.25-1.33 (m, 2H), 1.33-1.41 (m, 2H), 1.80-1.83 (m, |
| | 2H), 2.23-2.29 (m, 4H), 2.75-2.79 (m, 4H), 6.94-6.98 (m, 2H), |
| | 7.05-7.18 (m, 4H), 7.22-7.29 (m, 1H), 7.28 (br s, 1H), 7.33 (d, J = 8.0 Hz, 1H), |
| | 7.66 (br s, 1H), 7.93 (dd, J = 8.0 and 2.0, 1H), 8.01 (dd, J = 7.4 and 1.1 Hz, 1H), |
| | 8.15 (dd, J = 8.0 and 1.1 Hz, 1H), 8.23 (t, J = 7.9 Hz, 1H), 8.62 (d, J = 1.5 Hz, |
| | 1H), 11.64 (br s, 1H), 12.29 (br s, 1H). LCMS: M + H = 624.4. |
| 617 | LCMS: Single peak with M + H = 624.4 |
| 618 | LCMS: Single peak with M + H = 540.3 |
| 619 | LCMS: Single peak with M + H = 464.2 |
| 620 | 2.40 (s, 3H), 3.44 (s, 3H), 6.89 (d, 1H), 7.05 (br t, 1H), 7.24 (br t, 1H), |
| | 7.3 (br, 1H), 7.53 (t, 1H), 7.63 (d, 1H), 7.68 (br d, 1H), 7.82 (t, 1H), 7.86 (d, |
| | 1H), 8.01 (d, 1H), 8.42 (s, 1H), 10.6 (br, 1H). |
| 621 | 1.82-1.90 (m, 2H), 2.77-2.84 (m, 4H), 7.04 (t, 1H), 7.24 (t, 1H), |
| | 7.30-7.38 (br m, 3H), 7.57-7.68 (br m, 3H), 8.16 (dd, 1H), 8.32 (s, 1H). LCMS: |
| | M + H = 518.4 |
| 622 | 2.26 (s, 3H), 2.40 (s, 3H), 6.94 (d, 1H), 7.05 (t, 1H), 7.21-7.32 (m, 4H), |
| | 7.49 (t, 1H), 7.55 (d, 1H), 7.6-7.7 (br, 1H), 7.75 (t, 1H), 7.83-7.90 (m, 4H), |
| | 8.27 (s, 1H). LCMS: M + H = 514.3 |
| 623 | 2.41 (s, 3H), 7.05 (br t, 1H), 7.25 (br t, 1H), 7.3 (br, 1H), 7.45 (d, 1H), |
| | 7.58 (t, 1H), 7.63 (d, 1H), 7.65-7.71 (br, 1H), 7.78 (d, 1H), 8.02 (d, 1H), |
| | 8.10-8.18 (m, 2H). |
| 624 | 1.47 (t, 3H), 1.97 (t, 2H), 2.91 (m, 4H), 4.49 (q, 2H), 7.17 (t, 1H); 7.35 (t, |
| | 1H); 7.43 (d, 1H); 7.62-7.81 (m, 2H); 8.05-8.12 (m, 2H), 8.13-8.27 (m, |
| | 2H), 8.92 (s, 1H). MS: 443 (M + 1) |
| 625 | 1.09 (t, 3H), 2.90 (t, 2H), 3.31 (t, 2H), 3.46 (q, 2H), 6.89 (d, 1H), 7.03 (t, |
| | 1H), 7.24 (m, 2H), 7.67 (d, 1H), 7.86 (dd, 1H), 7.95-8.04 (m, 3H), 8.70 (s, |
| | 1H). MS: 444 (M + 1) |
| 626 | 2.83 (t, 2H), 3.27 (t, 2H), 6.55 (bs, 1H), 6.76 (d, 1H), 7.03 (t, 1H), 7.23 (m, |
| | 2H), 7.67 (d, 1H), 7.84 (dd, 1H), 7.91-8.01 (m, 3H), 8.60 (s, 1H). MS: |
| | 416 (M + 1) |
| 627 | 3.12 (m, 4H), 7.06 (t, 1H), 7.25 (m, 2H), 7.37 (d, 1H), 7.68 (d, 1H), |
| | 7.96-8.13 (m, 4H), 8.89 (s, 1H). MS: 433 (M + 1) |
| 628 | 1.68 (m, 4H), 2.77 (m, 4H), 7.03 (t, 1H), 7.24 (t, 1H), 7.32 (m, 2H), |
| | 7.65 (d, 1H), 7.96 (d, 1H), 8.05 (m, 2H), 8.14 (d, 1H), 8.19 (s, 1H). MS: |
| | 429 (M + 1) |
| 629 | 1.83-1.91 (m, 2H), 2.44-2.49 (m, 4H), 2.71 (t, 2H), 2.76-2.84 (m, 4H), |
| | 3.49 (m, 4H), 4.47 (t, 2H), 7.06 (t, 1H), 7.25 (t, 1H), 7.33 (br d, 2H), 7.70 (br d, |
| | 1H), 7.95-8.01 (m, 2H), 8.09 (t, 1H), 8.12 (dd, 1H), 8.78 (d, 1H). |
| 630 | 3.00-3.40 (m, 4H), 7.05-7.11 (m, 1H), 7.26-7.28 (m, 2H), 7.69 (d, J = 7.7 Hz, |
| | 1H), 7.87 (d, J = 7.8 Hz, 1H), 8.05 (dd, J = 7.6 and 1.1 Hz, 1H), 8.14 (t, |
| | J = 7.7 Hz, 1H), 8.22 (dd, J = 7.8 and 1.1 Hz, 1H), 8.30 (dd, J = 8.1 and 1.9 Hz, |
| | 1H), 8.93 (d, J = 1.8 Hz, 1H). LCMS: M + H = 449. |
| 631 | 3.40 (t, 2H), 3.73 (t, 2H), 7.08 (m, 1H), 7.25 (m, 2H), 7.69 (d, 1H), 7.97 (d, |
| | 1H), 8.08 (dd, 1H), 8.16 (t, 1H), 8.24 (dd, 1H), 8.35 (dd, 1H), 8.99 (s, 1H). |
| | LCMS: 465 (M + 1) |
| 632 | LCMS shows 2 isomers with M + H = 546 |
| 633 | 3.12-3.25 (m, 1H), 3.34-3.38 (m, 1H), 4.33-4.35 (m, 1H), 4.61 (br s, 1H), |
| | 6.41 (m, 1H), 6.99 (d, J = 8.6 Hz, 1H), 7.08-7.10 (m, 1H), 7.25-7.31 (m, |
| | 2H), 7.61 (d, J = 7.6 Hz, 1H), 8.02-8.05 (m, 1H), 8.10-8.14 (m, 2H), |
| | 8.70 (s, 1H). |
| 634 | 1.35 (s, 6H), 2.97 (s, 2H), 6.97 (d, J = 8.6 Hz, 1H), 7.02-7.07 (m, 1H), |
| | 7.25-7.24 (m, 2H), 7.66 (d, J = 7.9 Hz, 1H), 7.92-7.95 (m, 1H), |
| | 8.04-8.09 (m, 3H), 8.67 (d, J = 2.3 Hz, 1H). LCMS: M + H = 445.2. |
| 635 | 1.12 (d, 3H), 1.75-1.98 (br m, 2H), 2.7-2.8 (br m, 1H), 2.92-3.07 (br m, |
| | 1H), 3.6 (br s, 1H), 7.04 (br t, 1H), 7.2-7.3 (m, 2H), 7.32 (d, 1H), 7.67 (d, |
| | 1H), 7.97 (dd, 1H), 8.02-8.11 (m, 3H), 8.83 (d, 1H). |
| 636 | LCMS shows 2 isomers with M + H = 443 |
| 637 | 1.07 (d, 3H), 2.00 (m, 1H), 2.22 (dd, 1H), 2.53 (dd, 1H), 2.87 (dd, 1H), |
| | 3.12 (dd, 1H), 7.03 (t, 1H), 7.23 (t, 1H), 7.32 (d, 2H), 7.70 (d, 1H), |
| | 7.98-8.12 (m, 4H), 8.77 (s, 1H). MS: 429 (M + 1) |
| 638 | 2.03 (m, 2H), 2.95 (t, 2H), 4.17 (t, 2H), 7.04 (t, 1H), 7.10 (d, 1H), |
| | 7.21-7.31 (m, 2H), 7.65 (d, 1H), 7.95 (dd, 1H), 8.01-8.11 (m, 3H), 8.34 (s, 1H). |
| | MS: 431 (M + 1) |
| 639 | 3.22 (s, 3H), 7.17-7.20 (m, 2H), 7.32-7.41 (m, 2H), 7.72-7.74 (m, 1H), |
| | 7.93-7.95 (m, 1H), 8.04 (t, J = 7.7 Hz, 1H), 8.21-8.36 (m, 2H), 8.85 (s, |
| | 1H). LCMS: M + H = 430.3 |
| 700 | 2.37 (s, 3H), 7.04-7.09 (m, 2H), 7.25-7.26 (m, 2H), 7.33 (d, 1H), 7.44 (d, |
| | 1H), 7.53 (d, 1H), 7.72 (d, 1H) |
| 701 | 2.36 (s, 3H), 7.05-7.10 (m, 2H), 7.26-7.28 (m, 2H), 7.41 (d, 1H), 7.43 (d, |
| | 1H), 7.46 (d, 1H), 7.68-7.70 (m, 1H) |
| 800 | 1.86 (m, 2H), 2.79 (m, 4H), 7.04 (t, 1H), 7.25 (t, 1H), 7.33 (m, 2H), |
| | 7.72 (d, 1H), 7.86 (dd, 1H), 8.48 (s, 1H), 8.56 (s, 1H). MS: 421 (M + 1) |
| 801 | 1.85-1.87 (m, 2H), 2.79-2.82 (m, 4H), 7.03-7.09 (m, 1H), 7.22-7.34 (m, |
| | 3H), 7.37 (d, J = 8.0 Hz, 1 H), 7.73 (d, J = 8.1 Hz, 1H), 7.87 (dd, J = 8.0 |
| | and 1.9 Hz, 1H), 8.62 (s, 1H), 8.80 (s, 1H). |
| 802 | 3.00 (t, 2H), 4.33 (t, 2H), 7.02-7.08 (m, 2H), 7.25 (br d, 2H), 7.69 (d, 1H), |
| | 7.85 (dd, 1H), 8.44 (s, 1H), 8.48 (d, 1H). |
| 803 | 1.61 (br, 2H), 1.74 (br, 2H), 2.7-2.8 (br, 4H), 7.04 (t, 1H), 7.22 (t, 1H), |
| | 7.3-7.4 (m, 2H), 7.66 (d, 1H), 7.86 (d, 1H), 8.00 (s, 1H), 8.44 (s, 1H). |
| 804 | 1.90 (br t, 2H), 2.83 (br m, 4H), 7.09 (t, 1H), 7.28 (t, 1H), 7.32-7.41 (m, |
| | 2H), 7.74 (d, 1H), 7.89 (d, 1H), 8.34 (dd, 1H), 9.17 (d, 1H), 9.20 (s, 1H). |
| | LCMS: M + H = 416 |
| 900 | 2.44 (s, 3H), 6.94 (t, 1H), 7.13 (d, 1H), 7.20 (t, 1H), 7.55 (t, 1H), 7.64 (d, |
| | 1H), 7.89 (d, 1H), 7.99 (d, 1H), 8.08 (t, 1H), 8.16 (d, 1H), 8.21 (d, 1H), |
| | 8.54 (s, 1H). |
| 901 | 1.84-1.87 (m, 2H), 2.80-2.86 (m, 4H), 6.95 (t, 1H), 7.13 (d, 1H), 7.20 (t, |
| | 1H), 7.32 (d, 1H), 7.65 (d, 1H), 7.96-7.99 (dd, 1H), 8.03-8.12 9m, 3H), |
| | 8.81 (d, 1H). LCMS: 2 peaks of mass 463 (M + 1) |
| 902 | 3.12 (t, 2H), 3.18 (t, 2H), 6.96 (t, 1H), 7.11 (d, 1H), 7.21 (t, 1H), 7.36 (d, |
| | 1H), 7.66 (d, 1H), 7.96-8.13 (m, 4H), 8.93 (s, 1H). MS 481 (M + 1) |
| 1000 | LCMS: M + H: 431; >95% purity |
| 1001 | LCMS: M + H: 473; 85% purity. |
| 1002 | LCMS: M + H: 565; >95% purity. |
| 1003 | LCMS: M + H 445; >95% purity |
| 1100 | LCMS: 421 (M + 1) |
| 1101 | 1.91-2.04 (m, 2H), 2.4-2.85 (m, 4H), 6.96-7.08 (m, 1H), 7.13-7.32 (m, 2H), |
| | 7.50-7.70 (m, 2H), 7.84-7.91 (m, 1H), 8.03-8.21 (m, 1H), 8.34 and 8.44 (2 × bs, |
| | total 1H), 8.71 and 9.20 (2 × s, total 1H). LCMS: 2 peaks 404 (M + 1) |

### Biological Example 1

Measurement of competition of benzothiazole compounds with Bim26-mer for a Bcl-2 binding site.

Alphascreen (Amplified Luminescent Proximity Homogenous Assay) is a bead based technology which measures the interaction between molecules. The assay consists of two hydrogel coated beads which, when bought into close proximity by a binding interaction, allow the transfer of singlet oxygen from a donor bead to an acceptor bead.

Upon binding and excitation with laser light at 680 nm, a photosensitiser in the donor bead converts ambient oxygen to a more excited singlet state. This singlet oxygen then diffuses across to react with a chemiluminescer in the acceptor bead. Fluorophores within the same bead are activated resulting in the emission of light at 580-620 nm.

Screening of the benzothiazole test compounds was performed using the Alphascreen GST (glutathione s-transferase) detection kit system. Test compounds were titrated into the assay which consisted of GST tagged Bcl_{w} ΔC29 protein (0.05 nM Final concentration) and Biotinylated Bim BH3-26 peptide, Biotin-DLRPEIRIAQELRRIGDEFNETYTRR (3.0 nM Final concentration). For the GST tagged Bcl-x_{L} assay, GST tagged Bcl-x_{L} ΔC25 protein (0.6 nM Final concentration) and Biotinylated Bim BH3-26 peptide, Biotin-DLRPEIRIAQELRRIGDEFNETYTRR (5.0 nM Final concentration) were used. To this reaction mix anti-GST coated acceptor beads and Streptavidin coated donor beads, both at 15 μg/ml Final concentration, were added and the assay mixture incubated for 4 hours at room temperature before reading. Similarly when the Bcl-2 protein was Mcl-1, GST tagged Mcl-1 protein (0.4 nM Final concentration) and Biotinylated Bak BH3 peptide, Biotin-PSSTMGQVGRQLAIIGDDINRRYDSE-OH (4.0 nM Final concentration) were used.

Detailed Protocol:
1) prepare a 384 well with 4.75 μL of buffer and 0.25 μL of compounds (20 mM in DMSO) per well.
2) Mix the binding partners, in one tube add Bcl-w, Bcl-x_{L} or Mcl-1 and the acceptor beads, in the second tube add Biotinylated BH3 peptide and the donor beads.
3) Pre-incubate the two pairs of binding partners for 30 minutes.
4) Add 1 μL of acceptor beads:Bcl-w, Bcl-x_{L} or Mcl-1 protein mix to each well.
5) Seal the plate and incubate at room temperature for 30 minutes.
6) Add 10 μL of donor bead:BH3 peptide mix to each well.
7) Seal the plate, cover with foil and incubate for 4 hours.

Assay buffer contained 50 mM Hepes pH 7.4, 10 mM DTT, 100 mM NaCl, 0.05% Tween and 0.1 mg/ml casein. Bead dilution buffer contained 50 mM Tris, pH 7.5, 0.01% Tween and 0.1 mg/ml casein. The final DMSO concentration in the assay was 0.5%. Assays were performed in 384 well white Optiplates and analysed on the PerkinElmer Fusion alpha plate reader (Ex680, Em520-620 nM).

The GST Alphascreen detection kit and Optiplates were purchased from PerkinElmer.

Various compounds of the invention have IC₅₀ values between 1 nM and 10 μM for some of the Bcl-2 proteins. For example from Tables 1-11 the following compounds have IC₅₀ values for Bcl-xl which are below 10 μM: 1-2, 4-9, 11-14, 16, 18-22, 24-26, 33, 36, 39, 40, 200, 202, 206-208, 300, 302-309, 311-314, 316-331, 335-349, 358, 360-378, 380-389, 400, 500, 502, 503, 600, 601, 606-619, 621, 623-635, 637, 638, 700, 701, 800-803, 1000-1003 and 1100-1101.

### Biological Example 2

Cell Based Assay

The efficacy of the compounds of the present invention can also be determined in cell based killing assays using a variety of cell lines and mouse tumor models. For example, their activity on cell viability can be assessed on a panel of cultured tumorigenic and non-tumorigenic cell lines, as well as primary mouse or human cell populations, e.g. lymphocytes. For these assays, 5,000-20,000 cells are cultured at 37° C. and 10% CO₂ in appropriate growth media, eg: 100 μL Dulbecco's Modified Eagle's medium supplemented with 10% foetal calf serum, asparaginase and 2-mercaptoethanol in the case of pre-B Eμ-Myc mouse tumors in 96 well plates. Cell viability and total cell numbers can be monitored over 1-7 days of incubation with 1 nM-100 μM of the compounds to identify those that kill at IC50<10 μM. Cell viability is determined by the ability of the cells to exclude propidum iodide (10 μg/mL by immunofluorescence analysis of emission wavelengths of 660-675 nm on a flow cytometer (BD FACScan). Alternatively, a high throughput colorimetric assay such as the Cell Titre 96. AQueous Non-Radioactive Cell Proliferation Assay (Promega) may be used. Cell death by apoptosis is confirmed by pre-incubation of the cells with 50 μM of a caspase inhibitor such as zVAD-fmk. Drug internalisation is confirmed by confocal microscopy of conjugates labelled with a fluorochrome such as Fitc.

The conjugates of the present invention can also be evaluated for the specificity of their targets and mode of action in vivo. For example, if a conjugate comprises a compound of the invention that binds with high selectivity to Bcl-2, it should not kill cells lacking Bcl-2. Hence, the specificity of action can be confirmed by comparing the activity of the compound in wild-type cells with those lacking Bcl-2, derived from Bcl-2-deficient mice.

### Biological Example 3

Measurement of the affinity of the compounds of the invention for Bcl-xl protein was also determined using a competitive binding assay based on fluorescence polarization (FP). For this assay fluorescein labelled Bak peptide which is known to bind to the hydrophobic groove of Bcl-xl with high affinity was used following the general method described by [Wang et al., 2000]. Various compounds from Tables 1-5 were tested using the FP assay and for example compounds No. 2, 8, 12, 24, 305, 306, 308, 311, 319-324 and 400-401 gave IC₅₀ values for Bcl-xl which are below 1 μM.

### REFERENCES

A. Bajnati et al., Bull. Soc. Chim Fr., 1987, 318.

S. Cory, J. A. Adams, Cancer Cell, 2005, 5-6.

T. W. Green and P. Wuts, Protective Groups in Organic Synthesis, John Wiley & Sons, 3^{rd} Edition, 1999.

Y. Gong and K. Kato, J. Fluorine Chem., 2004, 767.

M. G. Hinds, M. Lackmann, G. L. Skea, P. J. Harrison, D. C. S. Huang, C. L. Day, EMBO J. 2003, 22, 1497.

K. T. Hodgetts and M. T. Kershaw, Org. Lett., 2002, 2905.

L. Katz, J. Am. Chem. Soc., 1951, 73, 4007.

X. Liu, S. Dai, Y. Zhu, P. Marrack, J. Kappler, Immunity. 2003, 19, 341.

S. W. Muchmore, M. Sattler, H. Liang, R. P. Meadows, J. E. Harlan, H. S. Yoon, D. Nettesheim, B. S. Chang, C. B. Thompson, S. L. Wong, S. L. Ng, S. W. Fesik, Nature. 1996, 381, 335.

Oleinik et. al., Russian Journal of Heterocyclic Chemistry, 1972, 402.

T. Oltersdorf, S. W. Elmore, A. R. Shoemaker, R.C. Armstrong, D. J. Augeri, B. A. Belli, M. Bruncko, T. L. Deckwerth, J. Dinges, P. J. Hajduk, M. K. Joseph, S. Kitada, S. J. Korsmeyer, A. R. Kunzer, A. Letai, C. Li, M. J. Mitten, D. G. Nettesheim, S. Ng, P. M. Nimmer, J. M. O'Connor, A. Oleksijew, A. M. Petros, J. C. Reed, W. Shen, S. K. Tahir, C. B. Thompson, K. J. Tomaselli, B. Wang, M. D. Wendt, H. Zhang, S. W. Fesik, S. H. Rosenberg, Nature, 2005, 435, 677-681.

G. A. Patani and E. J. LaVoie, Chem. Rev., 1996, 96, 3147-3176.

A. M. Petros, D. G. Nettesheim, Y. Wang, E. T. Olejniczak, R. P. Meadows, J. Mack, K. Swift, E. D. Matayoshi, H. Zhang, C. B. Thompson, S. W. Fesik, Protein Science. 2000, 9, 2528.

G. A. Reynolds et al., J. Org. Chem., 1959, 24, 1478.

M. Sattler, H. Liang, D. Nettesheim, R. P. Meadows, J. E. Harlan, M. Eberstadt, H. S. Yoon, S. B. Shuker, B. S. Chang, A. J. Minn, C. B. Thompson, S. W. Fesik, Science. 1997, 275, 983.

S. Sebille et al., J Med. Chem., 48, 614.

Wang et al., Proc. Nat. Acad. Sci., 2000, 97, 7124.

J. Y. Zhang, Nature Reviews/Drug Discovery 2002, 1, 101.

1. A compound of formula (I): 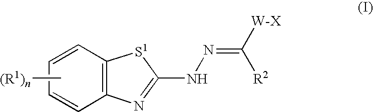
or a salt, ester or isomer thereof,
wherein
S¹ is S or Se;
W is an aryl or heteroaryl group;
X is -L-Z or -Q-Y—Z;
L is a linker of 1 to 3 atoms in length;
Q is an aryl or heteroaryl group;
Y is absent or is C₁₋₂alkylene;
Z is a carboxy group or an optionally substituted isosteric equivalent of a carboxy group;
R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyano and —N(R⁵)₂;
R² is selected from the group consisting of C₁₋₇alkyl, C₂₋₇alkenyl, C₂₋₇alkynyl, (CH₂)_{q}C₃₋₈cycloalkyl, (CH₂)_{q}halo, (CH₂)_{q}OH, C₁₋₇alkoxy(CH₂)ₜ, C₂₋₇alkenyloxy(CH₂)ₜ, C₂₋₇alkynyloxy(CH₂)ₜ, C₃₋₈cycloalkyloxy(CH₂)ₜ, C₁₋₇alkylthio(CH₂)ₜ, C₂₋₇alkenylthio(CH₂)ₜ, C₂₋₇alkynylthio(CH₂)ₜ, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl, or R² together with a ring atom from W forms an optionally substituted 5-8 membered carbocyclic or heterocyclic ring;
each R⁴ is independently selected from the group consisting of hydrogen and halogen;
each R⁵ is independently selected from the group consisting of hydrogen and C₁₋₃alkyl;
each R⁶ is independently selected from the group consisting of hydrogen, C₁₋₇alkyl, C₂₋₇alkenyl, C₂₋₇alkynyl, C₃₋₈cycloalkyl, acyl, aryl, heterocyclyl or heteroaryl, or two R⁶ taken together with the nitrogen atom to which they are attached form an optionally substituted carbocyclic or heterocyclic ring;
R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃₋₈cycloalkyloxy, aryloxy, heteroaryloxy, heterocyclyloxy and N(R⁶)₂;
M is O, S or NR⁵;
n is 0, 1 or 2;
q is 1 to 7;
r is 1 to 4;
t is 0 or 1 to 4;
wherein each alkyl, alkenyl, alkynyl, alkylene, linker, carbocyclic, heterocyclic, aryl and heteroaryl group may be optionally substituted with one or more optional substituents, provided that said compound is other than [4-[1-[(6-ethoxy-2-benzothiazolyl)hydrazono]ethyl]phenoxy]-acetic acid; [4-[1-[(6-methoxy-2-benzothiazolyl)hydrazono]ethyl]phenoxy]-acetic acid; [4-[1-[(6-methyl-2-benzothiazolyl)hydrazono]ethyl]phenoxy]-acetic acid; [4-[1-[(6-chloro-2-benzothiazolyl)hydrazono]ethyl]phenoxy]-acetic acid; and [4-[1-(2-benzothiazolylhydrazono)ethyl]phenoxy]-acetic acid.

2. A compound of formula (I) or a salt or isomer thereof of claim 1 wherein Z is an optionally substituted isosteric equivalent of a carboxy group selected from the group consisting of —CONHSO₂R³, —SO₂NHCOR³, —SO₂NHCONHR³, —SO₂NHR^{3a} and —NHSO₂R³, where R³ is selected from the group consisting of C₁₋₇alkyl, C₂₋₇alkenyl, C₂₋₇alkynyl, C₃₋₈cycloalkyl, aryl, heterocyclyl, heteroaryl, —(CH₂)_{q}C₃₋₈cycloalkyl, —(CH₂)_{q}halo, —(CH₂)_{q}OH, C₁₋₇alkoxy(CH₂)ₜ—, C₂₋₇alkenyloxy(CH₂)ₜ—, C₂₋₇alkynyloxy(CH₂)ₜ—, C₃₋₈cycloalkyloxy(CH₂)ₜ—, C₁₋₇alkylthio(CH₂)ₜ—, C₂₋₇alkenylthio(CH₂)ₜ—, C₂₋₇alkynylthio(CH₂)ₜ—, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl; and R^{3a} is an aryl or heteroaryl group.

3. A compound of formula (I) or a salt or isomer thereof of claim 1 or claim 2 in the form of an ester wherein Z is CO₂R³⁰, where R³⁰ is selected from the group consisting of C₁₋₇alkyl, C₂₋₇alkenyl, C₂₋₇alkynyl, C₃₋₈cycloalkyl, aryl, heterocyclyl, heteroaryl, —(CH₂)_{q}C₃₋₈cycloalkyl, —(CH₂)_{q}halo, —(CH₂)_{q}OH, C₁₋₇alkoxy(CH₂)_{q}—, C₂₋₇alkenyloxy(CH₂)_{q}—, C₂₋₇alkynyloxy(CH₂)_{q}—, C₃₋₈cycloalkyloxy(CH₂)_{q}—, C₁₋₇alkylthio(CH₂)_{q}—, C₂₋₇alkenylthio(CH₂)_{q}—, C₂₋₇alkynylthio(CH₂)_{q}—, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl, —(CH₂)ᵣM(CH₂)ₜheterocyclyl, —Si(C₁₋₆alkyl)₃ and —(CH₂)ᵣOSi(C₁₋₆alkyl)₃.

4. A compound according to claim 1 wherein W is furanyl, thiophenyl, pyrrolyl, N-methylpyrrolyl, pyrazolyl and phenyl, each of which may be optionally substituted.

5. A compound according to claim 1 wherein Q is selected from the group consisting of phenyl, pyridyl, furanyl, thiophenyl, pyrazolyl, pyrrolyl, N-methylpyrrolyl, thiazole, oxazole, triazole and pyrimidyl, each of which may be optionally substituted.

6. A compound according to claim 1 wherein R² is C₁₋₆alkyl or R² together with an atom in the aryl or heteroaryl ring of W forms an optionally substituted 5-8 membered carbocyclic or heterocyclic ring.

7. A compound according to claim 1 wherein R² and the carbon atom of W, together with atoms to which they are attached form an optionally substituted indanyl group, a tetrahydronaphthylene group, a chromanyl group, a tetrahydroquinolinyl or N-alkyl-tetrahydroquinolinyl group, a benzothiopyranyl group, a benzocycloheptenyl group, an S-oxido or S-dioxido-benzothiopyranyl group, a dihydroindolyl group or a 2-oxodihydroindolyl group.

8. A compound according to claim 1 wherein when W—X is W-Q-Y—Z, the attachment of Q to W is in a 1,3-arrangement with the attachment of W to the benzothiazole or benzoselenazole hydrazone moiety.

9. A compound according to claim 1 wherein when W—X is -W-Q-Y—Z, W and Y are positioned in a 1,3-arrangement with respect to Q.

10. A compound according to claim 1 wherein when W—X is W-L-Z, the attachment of L-Z to W is in a 1,4-arrangement with the attachment of W to the benzothiazole or benzoselenazole hydrazone moiety.

11. A compound according to claim 1 wherein L is a linker selected from the group consisting of —NH—, —CH₂—, —CH₂CH₂—, —OCH₂—, —SCH₂—, —NHCH₂—, —CH₂NH—, —O—CH(CH₃)— and —CH═CH—.

12. A compound according to claim 1 wherein the compound is of formula (IIa) 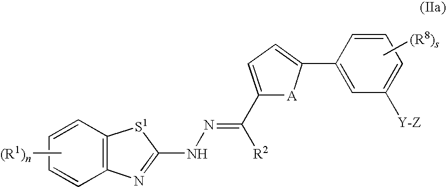
wherein
A is —O—, —S— or N(R⁵);
R⁸ is C₁₋₄alkyl, C₂₋₄alkenyl, C₂₋₄alkynyl, hydroxy, C₁₋₄alkoxy, C₁₋₄alkenyloxy, C₁₋₄alkynyloxy, C₂₋₄acyloxy, aryloxyC₁₋₄alkyloxy, halogen, —C(R⁴)₃, nitro, cyano, N(R⁵)₂, NHCOC₁₋₄alkyl, NHCOaryl, NHCOOC₁₋₄alkyl and NHCOC₁₋₄alkylaryl wherein each aryl group may be optionally substituted with methyl or methoxy;
s is 0, 1 or 2;
S¹, Y, Z, R¹, R², R⁴, R⁵, R⁶ and n are as defined for formula (I), or salts, esters or isomers thereof.

13. A compound according to claim 1 wherein the compound is of formula (IIb): 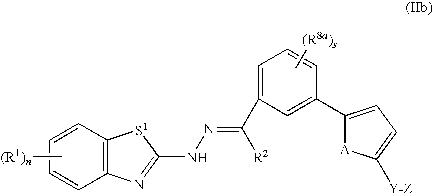
wherein S¹, Y, Z, R¹, R², R⁴, R⁵ and n are as defined for formula (I);
A is —O—, —S—, or N(R⁵);
s is 0, 1 or 2, and
R^{8a} is C₁₋₄alkyl, C₂₋₄alkenyl, C₂₋₄alkynyl, hydroxy, C₁₋₄alkoxy, C₁₋₄alkenyloxy, C₁₋₄alkynyloxy, halogen, —C(R⁴)₃, nitro, cyano and N(R⁵)₂;
or salts, esters or isomers thereof.

14. A compound according to claim 1 wherein the compound is of formula (IIc): 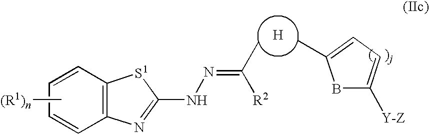
wherein S¹, Y, Z, R¹, R², R⁵ and n are as defined in formula (I);
H is a 5 or 6 membered heteroaryl group, wherein the benzothiazole hydrazone moiety and the aryl or heteroaryl group bearing the group Y—Z are bonded to group H in a 1,3 arrangement;
B is —O—, S, or —N(R⁵)— when j is 1, or
B is —N— or —CH— when j is 2, or salts, esters or isomers thereof

15. A compound according to claim 1 wherein the compound is of formula (IId): 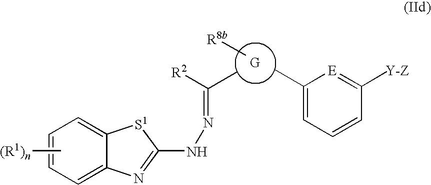
wherein S¹, Y, Z, R¹ and R² are as defined in formula (I);
G is a phenyl group or a 5 or 6 membered heteroaryl group, wherein the benzothiazole hydrazone moiety and the aryl or heteroaryl group bearing the group Y—Z are bonded to group G in a 1,3 arrangement;
E is —N— or —CH—;
R^{8b} is H or is R⁸ as defined in formula (IIa) in claim 12, or
R^{8b} and R² taken together form a 5 to 8 membered carbocyclic or heterocyclic ring, and salts, esters or isomers thereof.

16. A compound according to claim 1 wherein the compound is of formula (IIe): 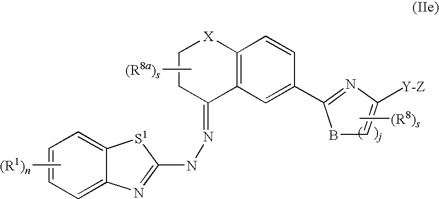
wherein S¹, Y, Z, R¹, R⁵ and n are as defined in formula (I);
X is —CH₂—, —CH₂CH₂— or —O—;
B is —O—, —S— or —NR⁵— when j is 1, or
B is —N— or —CH— when j is 2; and
R⁸ and s are as defined for formula (IIa) in claim 12,
R^{8a} is as defined for formula (IIb) in claim 13 or is oxo (═O),
or salts, esters or isomers thereof.

17. A compound according to claim 1 wherein the compound is of formula (IIIa): 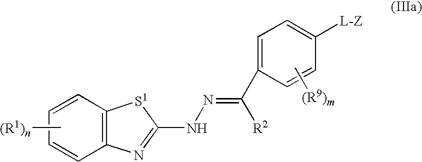
wherein S¹, L, Z, R¹, R² and n are as defined in formula (I); and
R⁹ is C₁₋₄alkyl, C₂₋₄alkenyl, C₂₋₄alkynyl, hydroxy, C₁₋₄alkoxy, C₁₋₄alkenyloxy, C₁₋₄alkynyloxy, halogen, —C(R⁴)₃, nitro, cyano and —N(R⁵)₂;
or when R⁹ and R² taken together form a 5 to 8 membered carbocyclic or heterocyclic ring, and m is 0, 1 or 2 or salts, esters or isomers thereof, provided that said compound is other than [4-[1-[(6-ethoxy-2-benzothiazolyl)hydrazono]ethyl]phenoxy]-acetic acid; [4-[1-[(6-methoxy-2-benzothiazolyl)hydrazono]ethyl]phenoxy]-acetic acid; [4-[1-[(6-methyl-2-benzothiazolyl)hydrazono]ethyl]phenoxy]-acetic acid; [4-[1-[(6-chloro-2-benzothiazolyl)hydrazono]ethyl]phenoxy]-acetic acid; and [4-[1-(2-benzothiazolylhydrazono)ethyl]phenoxy]-acetic acid.

18. A method of regulating the death of a cell comprising contacting the cell with an effective amount of a compound of formula (I): 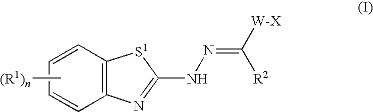
or a salt, ester or isomer thereof,
wherein
S¹ is S or Se;
W is an aryl or heteroaryl group;
X is -L-Z or -Q-Y—Z;
L is a linker of 1 to 3 atoms in length;
Q is an aryl or heteroaryl group;
Y is absent or is C₁₋₂alkylene;
Z is a carboxy group or an optionally substituted isosteric equivalent of a carboxy group;
R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyano and —N(R⁵)₂;
R² is selected from the group consisting of C₁₋₇alkyl, C₂₋₇alkenyl, C₂₋₇alkynyl, (CH₂)_{q}C₃₋₈cycloalkyl, (CH₂)_{q}halo, (CH₂)_{q}OH, C₁₋₇alkoxy(CH₂)ₜ, C₂₋₇alkenyloxy(CH₂)ₜ, C₂₋₇alkynyloxy(CH₂)ₜ, C₃₋₈cycloalkyloxy(CH₂)ₜ, C₁₋₇alkylthio(CH₂)ₜ, C₂₋₇alkenylthio(CH₂)ₜ, C₂₋₇alkynylthio(CH₂)ₜ, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl, or R² together with a ring atom from W forms an optionally substituted 5-8 membered carbocyclic or heterocyclic ring;
each R⁴ is independently selected from the group consisting of hydrogen and halogen;
each R⁵ is independently selected from the group consisting of hydrogen and C₁₋₃alkyl;
each R⁶ is independently selected from the group consisting of hydrogen, C₁₋₇alkyl, C₂₋₇alkenyl, C₂₋₇alkynyl, C₃₋₈cycloalkyl, acyl, aryl, heterocyclyl or heteroaryl, or two R⁶ taken together with the nitrogen atom to which they are attached form an optionally substituted carbocyclic or heterocyclic ring;
R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃₋₈cycloalkyloxy, aryloxy, heteroaryloxy, heterocyclyloxy and N(R⁶)₂;
M is O, S or NR⁵;
n is 0, 1 or 2;
q is 1 to 7;
r is 1 to 4;
t is 0 or 1 to 4;
wherein each alkyl, alkenyl, alkynyl, alkylene, linker, carbocyclic, heterocyclic, aryl and heteroaryl group may be optionally substituted with one or more optional substituents.

19. A method of inducing apoptosis in unwanted or damaged cells comprising contacting said damaged or unwanted cells with an effective amount of a compound of formula (I): 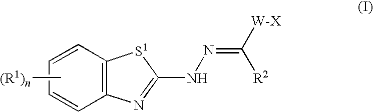
or a salt, ester or isomer thereof,
wherein
S¹ is S or Se;
W is an aryl or heteroaryl group;
X is -L-Z or -Q-Y—Z;
L is a linker of 1 to 3 atoms in length;
Q is an aryl or heteroaryl group;
Y is absent or is C₁₋₂alkylene;
Z is a carboxy group or an optionally substituted isosteric equivalent of a carboxy group;
R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyano and —N(R⁵)₂;
R² is selected from the group consisting of C₁₋₇alkyl, C₂₋₇alkenyl, C₂₋₇alkynyl, (CH₂)_{q}C₃₋₈cycloalkyl, (CH₂)_{q}halo, (CH₂)_{q}OH, C₁₋₇alkoxy(CH₂)ₜ, C₂₋₇alkenyloxy(CH₂)ₜ, C₂₋₇alkynyloxy(CH₂)ₜ, C₃₋₈cycloalkyloxy(CH₂)ₜ, C₁₋₇alkylthio(CH₂)ₜ, C₂₋₇alkenylthio(CH₂)ₜ, C₂₋₇alkynylthio(CH₂)ₜ, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CH, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl, or R² together with a ring atom from W forms an optionally substituted 5-8 membered carbocyclic or heterocyclic ring;
each R⁴ is independently selected from the group consisting of hydrogen and halogen;
each R⁵ is independently selected from the group consisting of hydrogen and C₁₋₃alkyl;
each R⁶ is independently selected from the group consisting of hydrogen, C₁₋₇alkyl, C₂₋₇alkenyl, C₂₋₇alkynyl, C₃₋₈cycloalkyl, acyl, aryl, heterocyclyl or heteroaryl, or two R⁶ taken together with the nitrogen atom to which they are attached form an optionally substituted carbocyclic or heterocyclic ring;
R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃₋₈cycloalkyloxy, aryloxy, heteroaryloxy, heterocyclyloxy and N(R⁶)₂;
M is O, S or NR⁵;
n is 0, 1 or 2;
q is 1 to 7;
r is 1 to 4;
t is 0 or 1 to 4;
wherein each alkyl, alkenyl, alkynyl, alkylene, linker, carbocyclic, heterocyclic, aryl and heteroaryl group may be optionally substituted with one or more optional substituents.

20. A method of treatment and/or prophylaxis of a pro-survival Bcl-2 member-mediated disease or condition in a mammal, comprising administering to said mammal an effective amount of a compound of formula (I): 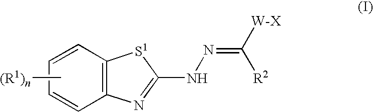
or a salt, ester or isomer thereof,
wherein
S¹ is S or Se;
W is an aryl or heteroaryl group;
X is -L-Z or -Q-Y—Z;
L is a linker of 1 to 3 atoms in length;
Q is an aryl or heteroaryl group;
Y is absent or is C₁₋₂alkylene;
Z is a carboxy group or an optionally substituted isosteric equivalent of a carboxy group;
R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyano and —N(R⁵)₂;
R² is selected from the group consisting of C₁₋₇alkyl, C₂₋₇alkenyl, C₂₋₇alkynyl, (CH₂)_{q}C₃₋₈cycloalkyl, (CH₂)_{q}halo, (CH₂)_{q}OH, C₁₋₇alkoxy(CH₂)ₜ, C₂₋₇alkenyloxy(CH₂)ₜ, C₂₋₇alkynyloxy(CH₂)ₜ, C₃₋₈cycloalkyloxy(CH₂)ₜ, C₁₋₇alkylthio(CH₂)ₜ, C₂₋₇alkenylthio(CH₂)ₜ, C₂₋₇alkynylthio(CH₂)ₜ, —(CH₂)_{q}aryl, —(CH₂)_{q}Oaryl, —(CH₂)_{q}Saryl, —(CH₂)_{q}heterocyclyl, (CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl, or R² together with a ring atom from W forms an optionally substituted 5-8 membered carbocyclic or heterocyclic ring;
each R⁴ is independently selected from the group consisting of hydrogen and halogen;
each R⁵ is independently selected from the group consisting of hydrogen and C₁₋₃alkyl;
each R⁶ is independently selected from the group consisting of hydrogen, C₁₋₇alkyl, C₂₋₇alkenyl, C₂₋₇alkynyl, C₃₋₈cycloalkyl, acyl, aryl, heterocyclyl or heteroaryl, or two R⁶ taken together with the nitrogen atom to which they are attached form an optionally substituted carbocyclic or heterocyclic ring;
R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃₋₈cycloalkyloxy, aryloxy, heteroaryloxy, heterocyclyloxy and N(R⁶)₂;
M is O, S or NR⁵;
n is 0, 1 or 2;
q is 1 to 7;
r is 1 to 4;
t is 0 or 1 to 4;
wherein each alkyl, alkenyl, alkynyl, alkylene, linker, carbocyclic, heterocyclic, aryl and heteroaryl group may be optionally substituted with one or more optional substituents.

21. A method according to claim 20 wherein the pro-survival Bcl-2 family member-mediated disease or condition is an inflammatory condition, benign or malignant cancer, hyperplasia, autoimmune disorders, tissue hypertrophy.

22. A method of treatment and/or prophylaxis of a disease or condition characterised by inappropriate persistence or proliferation of excess, unwanted or damaged cells in a mammal, comprising administering to said mammal an effective amount of a compound of formula (I): 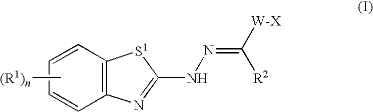
or a salt, ester or isomer thereof,
wherein
S¹ is S or Se;
W is an aryl or heteroaryl group;
X is -L-Z or -Q-Y—Z;
L is a linker of 1 to 3 atoms in length;
Q is an aryl or heteroaryl group;
Y is absent or is C₁₋₂alkylene;
Z is a carboxy group or an optionally substituted isosteric equivalent of a carboxy group;
R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyano —N(R⁵)₂;
R² is selected from the group consisting of C₁₋₇alkyl, C₂₋₇alkenyl, C₂₋₇alkynyl, (CH₂)_{q}C₃₋₈cycloalkyl, (CH₂)_{q}halo, (CH₂)_{q}OH, C₁₋₇alkoxy(CH₂)ₜ, C₂₋₇alkenyloxy(CH₂)ₜ, C₂₋₇alkynyloxy(CH₂)ₜ, C₃₋₈cycloalkyloxy(CH₂)ₜ, C₁₋₇alkylthio(CH₂)ₜ, C₂₋₇alkenylthio(CH₂)ₜ, C₂₋₇alkynylthio(CH₂)ₜ, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣM(CH₂)COR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl, or R² together with a ring atom from W forms an optionally substituted 5-8 membered carbocyclic or heterocyclic ring;
each R⁴ is independently selected from the group consisting of hydrogen and halogen;
each R⁵ is independently selected from the group consisting of hydrogen and C₁₋₃alkyl;
each R⁶ is independently selected from the group consisting of hydrogen, C₁₋₇alkyl, C₂₋₇alkenyl, C₂₋₇alkynyl, C₃₋₈cycloalkyl, acyl, aryl, heterocyclyl or heteroaryl, or two R⁶ taken together with the nitrogen atom to which they are attached form an optionally substituted carbocyclic or heterocyclic ring;
R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃₋₈cycloalkyloxy, aryloxy, heteroaryloxy, heterocyclyloxy and N(R⁶)₂;
M is O, S or NR⁵;
n is 0, 1 or 2;
q is 1 to 7;
r is 1 to 4;
t is 0 or 1 to 4;
wherein each alkyl, alkenyl, alkynyl, alkylene, linker, carbocyclic, heterocyclic, aryl and heteroaryl group may be optionally substituted with one or more optional substituents.

23. A method according to claim 22 wherein the disease or condition is B cell non-Hodgkin's lymphoma, B cell acute lymphoblastic leukaemia, B cell chronic lymphocytic leukaemia, follicular leukaemia, rheumatoid arthritis, systemic Lupus erythematosis, T cell acute lymphoblastic leukaemia, T cell non-Hodgkin's lymphoma, graft vs host disease, myelogenous leukaemia, chronic myelogenous leukaemia, chronic myelomonocytic leukaemia, multiple myeloma and cancer.

24. A method according to claim 22 wherein the excess, unwanted or damaged cells are unwanted or damaged B cells, T cells, myeloid cells, plasma cells, platelets, red blood cells, white blood cells and benign or malignant cancer cells.

25. A pharmaceutical composition comprising a compound of formula (I): 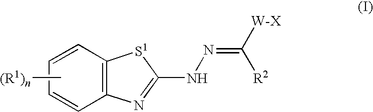
or a salt, ester or isomer thereof,
wherein
S¹ is S or Se;
W is an aryl or heteroaryl group;
X is -L-Z or -Q-Y—Z;
L is a linker of 1 to 3 atoms in length;
Q is an aryl or heteroaryl group;
Y is absent or is C₁₋₂alkylene;
Z is a carboxy group or an optionally substituted isosteric equivalent of a carboxy group;
R¹ is selected from the group consisting of halogen, hydroxy, C₁₋₃alkyl, C₂₋₃alkenyl, C₂₋₃alkynyl, C₁₋₃alkoxy, C₂₋₃alkenyloxy, C₂₋₃alkynyloxy, —C(R⁴)₃, —OC(R⁴)₃, nitro, cyano and —N(R⁵)₂;
R² is selected from the group consisting of C₁₋₇alkyl, C₂₋₇alkenyl, C₂₋₇alkynyl, (CH₂)_{q}C₃₋₈cycloalkyl, (CH₂)_{q}halo, (CH₂)_{q}OH, C₁₋₇alkoxy(CH₂)ₜ, C₂₋₇alkenyloxy(CH₂)ₜ, C₂₋₇alkynyloxy(CH₂)ₜ, C₃₋₈cycloalkyloxy(CH₂)ₜ, C₁₋₇alkylthio(CH₂)ₜ, C₂₋₇alkenylthio(CH₂)ₜ, C₂₋₇alkynylthio(CH₂)ₜ, —(CH₂)_{q}aryl, —(CH₂)_{q}Oaryl, —(CH₂)_{q}Saryl, —(CH₂)_{q}heterocyclyl, —(CH₂)_{q}heteroaryl, —(CH₂)_{q}C(R⁴)₃, —(CH₂)_{q}OC(R⁴)₃, —(CH₂)_{q}NO₂, —(CH₂)_{q}CN, —(CH₂)_{q}N(R⁶)₂, —(CH₂)_{q}CO₂H, —(CH₂)_{q}COR⁷, —(CH₂)ᵣM(CH₂)ᵣhalo, —(CH₂)ᵣM(CH₂)ᵣOH, —(CH₂)ᵣM(CH₂)ₜC(R⁴)₃, —(CH₂)ᵣM(CH₂)ᵣOC(R⁴)₃, —(CH₂)ᵣM(CH₂)ᵣNO₂, —(CH₂)ᵣM(CH₂)ᵣCN, —(CH₂)ᵣM(CH₂)ᵣN(R⁶)₂, —(CH₂)ᵣM(CH₂)ᵣCO₂H, —(CH₂)ᵣCOR⁷, —(CH₂)ᵣM(CH₂)ₜaryl, —(CH₂)ᵣM(CH₂)ₜheteroaryl, —(CH₂)ᵣM(CH₂)ₜcycloalkyl and —(CH₂)ᵣM(CH₂)ₜheterocyclyl, or R² together with a ring atom from W forms an optionally substituted 5-8 membered carbocyclic or heterocyclic ring;
each R⁴ is independently selected from the group consisting of hydrogen and halogen;
each R⁵ is independently selected from the group consisting of hydrogen and C₁₋₃alkyl;
each R⁶ is independently selected from the group consisting of hydrogen, C₁₋₇alkyl, C₂₋₇alkenyl, C₂₋₇alkynyl, C₃₋₈cycloalkyl, acyl, aryl, heterocyclyl or heteroaryl, or two R⁶ taken together with the nitrogen atom to which they are attached form an optionally substituted carbocyclic or heterocyclic ring;
R⁷ is selected from the group consisting of C₁₋₇alkyl, C₂₋₇alkenyl, C₂₋₇alkynyl, C₁₋₇alkoxy, C₂₋₇alkenyloxy, C₂₋₇alkynyloxy, C₃₋₈cycloalkyl, aryl, heteroaryl, C₃₋₈cycloalkyloxy, aryloxy, heteroaryloxy, heterocyclyloxy and N(R⁶)₂;
M is O, S or NR⁵;
n is 0, 1 or 2;
q is 1 to 7;
r is 1 to 4;
t is 0 or 1 to 4;
wherein each alkyl, alkenyl, alkynyl, alkylene, linker, carbocyclic, heterocyclic, aryl and heteroaryl group may be optionally substituted with one or more optional substituents and at least one pharmaceutically acceptable carrier.

26. (canceled)
